SEQUENCE LISTING (26 sequences)

SEQ ID NO: 1 - protein, 9 aa, synthetic construct
  REGION 1..9: note = Synthetic
GYSITSGYY

SEQ ID NO: 2 - protein, 7 aa, synthetic construct
  REGION 1..7: note = Synthetic
ITFDGAN

SEQ ID NO: 3 - protein, 12 aa, synthetic construct
  REGION 1..12: note = Synthetic
TRSSYDYDVLDY

SEQ ID NO: 4 - protein, 6 aa, synthetic construct
  REGION 1..6: note = Synthetic
QDISNF

SEQ ID NO: 5 - skipped

SEQ ID NO: 6 - protein, 9 aa, synthetic construct
  REGION 1..9: note = Synthetic
QQGHTLPYT

SEQ ID NO: 7 - protein, 6 aa, synthetic construct
  REGION 1..6: note = Synthetic
SGYYWN

SEQ ID NO: 8 - protein, 16 aa, synthetic construct
  REGION 1..16: note = Synthetic
YITFDGANNYNPSLKN

SEQ ID NO: 9 - protein, 10 aa, synthetic construct
  REGION 1..10: note = Synthetic
SSYDYDVLDY

SEQ ID NO: 10 - protein, 11 aa, synthetic construct
  REGION 1..11: note = Synthetic
RASQDISNFLN

SEQ ID NO: 11 - protein, 7 aa, synthetic construct
  REGION 1..7: note = Synthetic
YTSRLHS

SEQ ID NO: 12 - protein, 8 aa, synthetic construct
  REGION 1..8: note = Synthetic
GYSITSGY

SEQ ID NO: 13 - skipped

SEQ ID NO: 14 - protein, 8 aa, synthetic construct
  REGION 1..8: note = Synthetic
SYDYDVLD

SEQ ID NO: 15 - protein, 7 aa, synthetic construct
  REGION 1..7: note = Synthetic
SQDISNF

SEQ ID NO: 16 - protein, 6 aa, synthetic construct
  REGION 1..6: note = Synthetic
GHTLPY

SEQ ID NO: 17 - protein, 119 aa, synthetic construct
  REGION 1..119: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSS

SEQ ID NO: 18 - protein, 107 aa, synthetic construct
  REGION 1..107: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIK

SEQ ID NO: 19 - protein, 227 aa, synthetic construct
  REGION 1..227: note = Synthetic
QVQLQESGPGLVKPSQTLSLTCTVTGYSITSGYYWNWIRQPPGKGLEWIGYITFDGANNYNPSLKNRVSISRDTSKNQFSLKLSSVTAEDTATYYCTRSSYDYDVLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 20 - protein, 214 aa, synthetic construct
  REGION 1..214: note = Synthetic
DIQMTQSPSSLSASVGDRVTITCRASQDISNFLNWYQQKPGQPVKLLIYYTSRLHSGVPSRFSGSGSGTDFTLTISSLQPEDFATYYCQQGHTLPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - RNA, 16 nt, synthetic construct
  misc_feature 1..16: note = Synthetic
  source 1..16: mol_type = other RNA
cagcgcccaccagtca

SEQ ID NO: 22 - DNA, 16 bp, synthetic construct
  misc_feature 1..16: note = Synthetic
  source 1..16: mol_type = other DNA
tgactggtgggcgctg

SEQ ID NO: 23 - protein, 760 aa, Homo sapiens
MMDQARSAFSNLFGGEPLSYTRFSLARQVDGDNSHVEMKLAVDEEENADNNTKANVTKPKRCSGSICYGTIAVIVFFLIGFMIGYLGYCKGVEPKTECERLAGTESPVREEPGEDFPAARRLYWDDLKRKLSEKLDSTDFTGTIKLLNENSYVPREAGSQKDENLALYVENQFREFKLSKVWRDQHFVKIQVKDSAQNSVIIVDKNGRLVYLVENPGGYVAYSKAATVTGKLVHANFGTKKDFEDLYTPVNGSIVIVRAGKITFAEKVANAESLNAIGVLIYMDQTKFPIVNAELSFFGHAHLGTGDPYTPGFPSFNHTQFPPSRSSGLPNIPVQTISRAAAEKLFGNMEGDCPSDWKTDSTCRMVTSESKNVKLTVSNVLKEIKILNIFGVIKGFVEPDHYVVVGAQRDAWGPGAAKSGVGTALLLKLAQMFSDMVLKDGFQPSRSIIFASWSAGDFGSVGATEWLEGYLSSLHLKAFTYINLDKAVLGTSNFKVSASPLLYTLIEKTMQNVKHPVTGQFLYQDSNWASKVEKLTLDNAAFPFLAYSGIPAVSFCFCEDTDYPYLGTTMDTYKELIERIPELNKVARAAAEVAGQFVIKLTHDVELNLDYERYNSQLLSFVRDLNQYRADIKEMGLSLQWLYSARGDFFRATSRLTTDFGNAEKTDRFVMKKLNDRVMRVEYHFLSPYVSPKESPFRHVFWGSGSHTLPALLENLKLRKQNNGAFNETLFRNQLALATWTIQGAANALSGDVWDIDNEF

SEQ ID NO: 24 - DNA, 2859 bp, Homo sapiens
  source 1..2859: mol_type = genomic DNA
aggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtgggcggagcccatcgtggtgaggcttaaggaggtccgactgcagagggacgacttcgagattctgaaggtgatcggacgcggggcgttcagcgaggtagcggtagtgaagatgaagcagacgggccaggtgtatgccatgaagatcatgaacaagtgggacatgctgaagaggggcgaggtgtcgtgcttccgtgaggagagggacgtgttggtgaatggggaccggcggtggatcacgcagctgcacttcgccttccaggatgagaactacctgtacctggtcatggagtattacgtgggcggggacctgctgacactgctgagcaagtttggggagcggattccggccgagatggcgcgcttctacctggcggagattgtcatggccatagactcggtgcaccggcttggctacgtgcacagggacatcaaacccgacaacatcctgctggaccgctgtggccacatccgcctggccgacttcggctcttgcctcaagctgcgggcagatggaacggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggagacactgtcggacattcgggaaggtgcgccgctaggggtccacctgccttttgtgggctactcctactcctgcatggccctcagggacagtgaggtcccaggccccacacccatggaactggaggccgagcagctgcttgagccacacgtgcaagcgcccagcctggagccctcggtgtccccacaggatgaaacagctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccagtcaactacgcgaggcagaggctcggaaccgggacctagaggcacacgtccggcagttgcaggagcggatggagttgctgcaggcagagggagccacagctgtcacgggggtccccagtccccgggccacggatccaccttcccatctagatggccccccggccgtggctgtgggccagtgcccgctggtggggccaggccccatgcaccgccgccacctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctgga

SEQ ID NO: 25 - DNA, 2683 bp, Mus musculus
  source 1..2683: mol_type = genomic DNA
gaactggccagagagacccaagggatagtcagggacgggcagacatgcagctagggttctggggcctggacaggggcagccaggccctgtgacgggaagaccccgagctccggcccggggaggggccatggtgttgcctgcccaacatgtcagccgaagtgcggctgaggcagctccagcagctggtgctggacccaggcttcctgggactggagcccctgctcgaccttctcctgggcgtccaccaggagctgggtgcctctcacctagcccaggacaagtatgtggccgacttcttgcagtgggtggagcccattgcagcaaggcttaaggaggtccgactgcagagggatgattttgagattttgaaggtgatcgggcgtggggcgttcagcgaggtagcggtggtgaagatgaaacagacgggccaagtgtatgccatgaagattatgaataagtgggacatgctgaagagaggcgaggtgtcgtgcttccgggaagaaagggatgtattagtgaaaggggaccggcgctggatcacacagctgcactttgccttccaggatgagaactacctgtacctggtcatggaatactacgtgggcggggacctgctaacgctgctgagcaagtttggggagcggatccccgccgagatggctcgcttctacctggccgagattgtcatggccatagactccgtgcaccggctgggctacgtgcacagggacatcaaaccagataacattctgctggaccgatgtgggcacattcgcctggcagacttcggctcctgcctcaaactgcagcctgatggaatggtgaggtcgctggtggctgtgggcaccccggactacctgtctcctgagattctgcaggccgttggtggagggcctggggcaggcagctacgggccagagtgtgactggtgggcactgggcgtgttcgcctatgagatgttctatgggcagacccccttctacgcggactccacagccgagacatatgccaagattgtgcactacagggaacacttgtcgctgccgctggcagacacagttgtccccgaggaagctcaggacctcattcgtgggctgctgtgtcctgctgagataaggctaggtcgaggtggggcagacttcgagggtgccacggacacatgcaatttcgatgtggtggaggaccggctcactgccatggtgagcgggggcggggagacgctgtcagacatgcaggaagacatgccccttggggtgcgcctgcccttcgtgggctactcctactgctgcatggccttcagagacaatcaggtcccggaccccacccctatggaactagaggccctgcagttgcctgtgtcagacttgcaagggcttgacttgcagcccccagtgtccccaccggatcaagtggctgaagaggctgacctagtggctgtccctgcccctgtggctgaggcagagaccacggtaacgctgcagcagctccaggaagccctggaagaagaggttctcacccggcagagcctgagccgcgagctggaggccatccggaccgccaaccagaacttctccagccaactacaggaggccgaggtccgaaaccgagacctggaggcgcatgttcggcagctacaggaacggatggagatgctgcaggccccaggagccgcagccatcacgggggtccccagtccccgggccacggatccaccttcccatctagatggccccccggccgtggctgtgggccagtgcccgctggtggggccaggccccatgcaccgccgtcacctgctgctccctgccaggatccctaggcctggcctatccgaggcgcgttgcctgctcctgttcgccgctgctctggctgctgccgccacactgggctgcactgggttggtggcctataccggcggtctcaccccagtctggtgtttcccgggagccaccttcgccccctgaaccctaagactccaagccatctttcatttaggcctcctaggaaggtcgagcgaccagggagcgacccaaagcgtctctgtgcccatcgcgccccccccccccccccaccgctccgctccacacttctgtgagcctgggtccccacccagctccgctcctgtgatccaggcctgccacctggcggccggggagggaggaacagggctcgtgcccagcacccctggttcctgcagagctggtagccaccgctgctgcagcagctgggcattcgccgaccttgctttactcagccccgacgtggatgggcaaactgctcagctcatccgatttcactttttcactctcccagccatcagttacaagccataagcatgagccccctatttccagggacatcccattcccatagtgatggatcagcaagacctctgccagcacacacggagtctttggcttcggacagcctcactcctgggggttgctgcaactccttccccgtgtacacgtctgcactctaacaacggagccacagctgcactcccccctcccccaaagcagtgtgggtatttattgatcttgttatctgactcactgacagactccgggacccacgttttagatgcattgagactcgacattcctcggtatttattgtctgtccccacctacgacctccactcccgacccttgcgaataaaatacttctggtctgccctaaa

SEQ ID NO: 26 - DNA, 19841 bp, Homo sapiens
  source 1..19841: mol_type = genomic DNA
tttcctacttgaagcctgacgtagtaaagatcgggggagggttagacagatacagtggtccccaaccattttggcaccagggactggtcttatggaagacagtttttccacagactgttggggggatggttttgggatgaaaccgttctgcctctgatcatcaggtgttagattctaataaggagcgcacacctagatccctcgcatgcatagttcatggtggggttcgcactcttacgaggattgaatggtgcgctgctccggtaggaggctgggctcaggctgtaatgcctgctcgcccaccactcacctcctgctgcatggcctggttcctaacaggccacggaccactactgttccatggcccggaggttgaggaccccgagatacaggacaattctgtggcaagcaggactgtcccctcgccaaagatgggacatcgaggctccttggagcaccctgtggccaccttgcagcagcctctgtttccccatgtttccatgacctggtgtccatctgtcttccccagtttgggagcttctctccgaggaggacctgggcctggtgtggcacctgctgtgtgagcggggccatgtccaacggccttcttggggactttgggtcggggagaagttctgcctgggttttactgccttctcccaaccccacactgtctcccctggcagcggttgatcgacaagaccaaggtgacatatctgaagtggctgcctgagtcggagagcctgttcctggcatcacacgccagtggccacctgtacctgtacaacgtcagccacccctgcgcctcggccccgccccagtacagcctgctgaagcagggcgagggcttctctgtctatgctgccaagagcaaggcaccccgcaacccgctggccaagtgggcggtgggtgaggggcccctcaacgagttcgccttctcgcccgatggccggcacctggcctgtgtgagccaggatggctgcctgcgcgtcttccacttcgactccatgctcctgcgtgggctcatgaagagctactttgggggcctgctgtgtgtgtgctggagccctgacggccgctacgtggtgacgggtggcgaagatgacctggtcaccgtgtggtccttcaccgagggccgcgtggtggctcgaggccatggccacaagtcctgggtcaacgctgtggcctttgacccctacaccacaagggcagaggaggcggcgacagcagccggtgctgatggggagcggagcggcgaagaggaggaggaggagcccgaggctgcgggcacaggctcggccgggggcgccccgctctctccactgcccaaggctggctccattacttaccgctttggctcggcgggccaggacacgcagttctgcctgtgggacctcactgaagacgtgctctacccgcacccccccctggcccgcacccgcaccctccctggcacacctggcaccacgccaccggccgccagcagctcgaggggtggcgagcctggcccaggccccctgcctcgctcgctgtcccgctccaacagtctcccgcacccagctggcgggggcaaggcgggcggcccgggtgtggcggcagagcctggcacaccattcagcattggccgcttcgccacgctcacactgcaggagcggcgggaccggggggcagagaaggagcacaagcgctaccacagcctgggcaacatcagccggggtggcagtggcggcagtggcagtggtggggagaagcccagcggccctgttccccgcagccgcctggaccccgccaaggtgctgggcactgcgctgtgcccgcgcatccacgaggtgcccctgctggagccccttgtgtgcaagaagatcgcccaggagcggctcacagtcctcctgttcctggaggactgcatcatcactgcctgccaggagggcctcatctgcacctgggcccggccgggcaaggcggtgagtggccccacaccagcctgccggggacctggcaggacctttcgtgggaagaggcaggcattggcagagagagggctttgttgctgtcacagcctctggctccgtggggtgaggggaagccagggaaatcttagtgtctcagtacaagacctctcagatccttagagtgagggggtctagccctaggcagcaggcagcagaaagaggggtgggtgtgagagccagctaggaattggggcatccaaggctggccgtctgaagggcagcagatgggccccacatggccaggtcttactgcctgtcactcgaaccagaatctatttctgttgaacatctgttttttaaatcgtgaaacttttttgagtacttcaggccaaaactaggggcgagctcaagcctgtgggcatggctgccagcctgggtctgggactcaggatctgagcctcctgctgaaggcacaggctgggaatcccaggcctgggttccagtcccactccctctgtgaccctggacaagtcactgccccctctgacctccaactcatcacctcttagaacagagcctgtaggatgggcagtgggtggatgtgcttgcctcctgggtgggctgtggcgttgggaaggtcatagtaggcgaatcaggcctggcatcttgtaagttcggagctcgtcttgggtgtctcagcttcttagggcttggactcagttgcccagggtcctggaggccgtggcttggttcctcagatcctcagttttggaatcgtagagtcctgagtccctagaacttgagagcacagtctgagtgactcagaggcaagagtggtgggatttggggagtctggttgagtcctaaaagagacccctctgtctccgtagttcacagacgaggagaccgaggcccagacaggggaaggaagttggcccaggtcacccagcaagtcagtggtagaggtaggactgtccctgagttctttccccagcacctcagggtccctcccaagttagaagggagctccagtttccccctcccctcccacccttacccttaccccatggtctcactcaggatccgccaaggactttgattattgcgtgaaagtgctgactgccaggacaggaagctagctaagatgcaagttcccagcctagagcagtggcctctggggggtctagggcggacccaagggcaaggccagggtggcagcagctttggggactctgggctggctccctccccttgacactggctgaagcccaggtggtctctaacccctcccatctctccctctcatcttccccagggcatctcctcccaaccaggcaactccccgagtggcacagtggtgtgaagccatggatatcgggcccccccaaccccatgcccccagcctcctagccataaccctccctgctgacctcacagatcaacgtattaacaagactaaccatgatggatggactgctccagtccccccacctgcacaaaatttgggggccccccagactggcccggacacgggcgatgtaatagcccttgtggcctcagccttgtcccccacccactgccaagtacaatgacctcttcctctgaaacatcagtgttaccctcatccctgtccccagcatgtgactggtcactcctggggagagactccccgcccctgccacaagagccccaggtctgcagtgtgcccctcagttgagtgggcagggccgggggtggtccagccctcgcccggcccccaccccagctgcccttgctattgtctgtgcttttgaagagtgttaaattatggaagcccctcaggttcctccctgtcccgcaggacctcttatttatactaaagttccctgttttctcagcgggtctgtccccttcggaggagatgatgtagaggacctgtgtgtgtactctgtggttctaggcagtccgctttccccagaggaggagtgcaggcctgctcccagcccagcgcctcccaccccttttcatagcaggaaaagccggagcccagggagggaacggacctgcgagtcacacaactggtgacccacaccagcggctggagcaggaccctcttggggagaagagcatcctgcccgcagccagggcccctcatcaaagtcctcggtgttttttaaattatcagaactgcccaggaccacgtttcccaggccctgcccagctgggactcctcggtccttgcctcctagtttctcaggcctggccctctcaaggcccaggcaccccaggccggttggaggccccgacttccactctggagaaccgtccaccctggaaagaagagctcagattcctcttggctctcggagccgcagggagtgtgtcttcccgcgccaccctccaccccccgaaatgtttctgtttctaatcccagcctgggcaggaatgtggctccccggccaggggccaaggagctattttggggtctcgtttgcccagggagggcttggctccaccactttcctcccccagcctttgggcagcaggtcacccctgttcaggctctgagggtgccccctcctggtcctgtcctcaccaccccttccccacctcctgggaaaaaaaaaaaaaaaaaaaaaaaaagctggtataaagcagagagcctgagggctaaatttaactgtccgagtcggaatccatctctgagtcacccaagaagctgccctggcctcccgtccccttcccaggcctcaacccctttctcccacccagccccaacccccagccctcaccccctagcccccagttctggagcttgtcgggagcaagggggtggttgctactgggtcactcagcctcaattggccctgtttcagcaatgggcaggttcttcttgaaattcatcacacctgtggcttcctctgtgctctacctttttattggggtgacagtgtgacagctgagattctccatgcattccccctactctagcactgaagggttctgaagggccctggaaggagggagcttggggggctggcttgtgaggggttaaggctgggaggcgggaggggggctggaccaaggggtggggagaaggggaggaggcctcggccggccgcagagagaagtggccagagaggcccaggggacagccagggacaggcagacatgcagccagggctccagggcctggacaggggctgccaggccctgtgacaggaggaccccgagcccccggcccggggaggggccatggtgctgcctgtccaacatgtcagccgaggtgcggctgaggcggctccagcagctggtgttggacccgggcttcctggggctggagcccctgctcgaccttctcctgggcgtccaccaggagctgggcgcctccgaactggcccaggacaagtacgtggccgacttcttgcagtggggtgagtgcctaccctcggggctcctgcagatggggtgggggtggggcaggagacaggtctgggcacagaggcctggctgttgggggggcaggatggcaggatgggcatggggagatcctcccatcctggggctcagagtgtggacctgggccctggggcaacatttctctgtcctatgccaccactctggaggggcagagtaaggtcagcagaggctagggtggctgtgactcagagccatggcttaggagtcacagcaggctaggctgccaacagcctcccatggcctctctgcaccccgcctcagggtcagggtcagggtcatgctgggagctccctctcctaggaccctccccccaaaagtgggctctatggccctctcccctggtttcctgtggcctggggcaagccaggagggccagcatggggcagctgccaggggcgcagccgacaggcaggtgttcggcgccagcctctccagctgccccaacaggtgcccaggcactgggagggcggtgactcacgcgggccctgtgggagaaccagctttgcagacaggcgccaccagtgccccctcctctgcgatccaggagggacaactttgggttcttctgggtgtgtctccttcttttgtaggttctgcacccacccccacccccagccccaaagtctcggttcctatgagccgtgtgggtcagccaccattcccgccaccccgggtccctgcgtcctttagttctcctggcccagggcctccaaccttccagctgtcccacaaaaccccttcttgcaagggctttccagggcctggggccagggctggaaggaggatgcttccgcttctgccagctgccttgtctgcccacctcctccccaagcccaggactcgggctcactggtcactggtttctttcattcccagcaccctgcccctctggccctcatatgtctggccctcagtgactggtgtttggtttttggcctgtgtgtaacaaactgtgtgtgacacttgtttcctgtttctccgccttcccctgcttcctcttgtgtccatctctttctgacccaggcctggttcctttccctcctcctcccatttcacagatgggaaggtggaggccaagaagggccaggccattcagcctctggaaaaaccttctcccaacctcccacagcccctaatgactctcctggcctccctttagtagaggatgaagttgggttggcagggtaaactgagaccgggtggggtaggggtctggcgctcccgggaggagcactccttttgtggcccgagctgcatctcgcggcccctcccctgccaggcctggggcgggggagggggccagggttcctgctgccttaaaagggctcaatgtcttggctctctcctccctcccccgtcctcagccctggctggttcgtccctgctggcccactctcccggaaccccccggaacccctctctttcctccagaacccactgtctcctctccttccctcccctcccatacccatccctctctccatcctgcctccacttcttccacccccgggagtccaggcctccctgtccccacagtccctgagccacaagcctccaccccagctggtcccccacccaggctgcccagtttaacattcctagtcataggaccttgacttctgagaggcctgattgtcatctgtaaataaggggtaggactaaagcactcctcctggaggactgagagatgggctggaccggagcacttgagtctgggatatgtgaccatgctacctttgtctccctgtcctgttccttcccccagccccaaatccagggttttccaaagtgtggttcaagaaccacctgcatctgaatctagaggtactggatacaaccccacgtctgggccgttacccaggacattctacatgagaacgtgggggtggggccctggctgcacctgaactgtcacctggagtcagggtggaaggtggaagaactgggtcttatttccttctccccttgttctttagggtctgtccttctgcagactccgttaccccaccctaaccatcctgcacacccttggagccctctgggccaatgccctgtcccgcaaagggcttctcaggcatctcacctctatgggagggcatttttggcccccagaaccttacacggtgtttatgtggggaagcccctgggaagcagacagtcctagggtgaagctgagaggcagagagaaggggagacagacagagggtggggctttcccccttgtctccagtgccctttctggtgaccctcggttcttttcccccaccacccccccagcggagcccatcgtggtgaggcttaaggaggtccgactgcagagggacgacttcgagattctgaaggtgatcggacgcggggcgttcagcgaggtaagccgaaccgggcgggagcctgacttgactcgtggtgggcggggcataggggttggggcggggccttagaaattgatgaatgaccgagccttagaacctagggctgggctggaggcggggcttgggaccaatgggcgtggtgtggcaggtggggcggggccacggctgggtgcagaagcgggtggagttgggtctgggcgagcccttttgttttcccgccgtctccactctgtctcactatctcgacctcaggtagcggtagtgaagatgaagcagacgggccaggtgtatgccatgaagatcatgaacaagtgggacatgctgaagaggggcgaggtgaggggctgggcggacgtggggggctttgaggatccgcgccccgtctccggctgcagctcctccgggtgccctgcaggtgtcgtgcttccgtgaggagagggacgtgttggtgaatggggaccggcggtggatcacgcagctgcacttcgccttccaggatgagaactacctggtgagctccgggccggggtgactaggaagagggacaagagcccgtgctgtcactggacgaggaggtggggagaggaagctctaggattgggggtgctgcccggaaacgtctgtgggaaagtctgtgtgcggtaagagggtgtgtcaggtggatgaggggccttccctatctgagacggggatggtgtccttcactgcccgtttctggggtgatctgggggactcttataaagatgtctctgttgcggggggtctcttacctggaatgggataggtcttcaggaattctaacggggccactgcctagggaaggagtgtctgggacctattctctgggtgttgggtggcctctgggttctctttcccagaacatctcagggggagtgaatctgcccagtgacatcccaggaaagtttttttgtttgtgtttttttttgaggggcgggggcgggggccgcaggtggtctctgatttggcccggcagatctctatggttatctctgggctggggctgcaggtctctgcccaaggatggggtgtctctgggaggggttgtcccagccatccgtgatggatcagggcctcaggggactaccaaccacccatgacgaaccccttctcagtacctggtcatggagtattacgtgggcggggacctgctgacactgctgagcaagtttggggagcggattccggccgagatggcgcgcttctacctggcggagattgtcatggccatagactcggtgcaccggcttggctacgtgcacaggtgggtgcagcatggccgaggggatagcaagcttgttccctggccgggttcttggaaggtcagagcccagagaggccagggcctggagagggaccttcttggttggggcccaccggggggtgcctgggagtaggggtcagaactgtagaagccctacaggggcggaacccgaggaagtggggtcccaggtggcactgcccggaggggcggagcctggtgggaccacagaagggaggttcatttatcccacccttctcttttcctccgtgcagggacatcaaacccgacaacatcctgctggaccgctgtggccacatccgcctggccgacttcggctcttgcctcaagctgcgggcagatggaacggtgagccagtgccctggccacagagcaactggggctgctgatgagggatggaaggcacagagtgtgggagcgggactggatttggaggggaaaagaggtggtgtgacccaggcttaagtgtgcatctgtgtggcggagtattagaccaggcagagggaggggctaagcatttggggagtggttggaaggagggcccagagctggtgggcccagaggggtgggcccaagcctcgctctgctccttttggtccaggtgcggtcgctggtggctgtgggcaccccagactacctgtcccccgagatcctgcaggctgtgggcggtgggcctgggacaggcagctacgggcccgagtgtgactggtgggcgctgggtgtattcgcctatgaaatgttctatgggcagacgcccttctacgcggattccacggcggagacctatggcaagatcgtccactacaaggtgagcacggccgcagggagacctggcctctcccggtaggcgctcccaggctatcgcctcctctccctctgagcaggagcacctctctctgccgctggtggacgaaggggtccctgaggaggctcgagacttcattcagcggttgctgtgtcccccggagacacggctgggccggggtggagcaggcgacttccggacacatcccttcttctttggcctcgactgggatggtctccgggacagcgtgcccccctttacaccggatttcgaaggtgccaccgacacatgcaacttcgacttggtggaggacgggctcactgccatggtgagcgggggcggggtaggtacctgtggcccctgctcggctgcgggaacctccccatgctccctccataaagttggagtaaggacagtgcctaccttctggggtcctgaatcactcattccccagagcacctgctctgtgcccatctactactgaggacccagcagtgacctagacttacagtccagtgggggaacacagagcagtcttcagacagtaaggccccagagtgatcagggctgagacaatggagtgcagggggtgggggactcctgactcagcaaggaaggtcctggagggctttctggagtggggagctatctgagctgagacttggagggatgagaagcaggagaggactcctcctcccttaggccgtctctcttcaccgtgtaacaagctgtcatggcatgcttgctcggctctgggtgcccttttgctgaacaatactggggatccagcacggaccagatgagctctggtccctgccctcatccagttgcagtctagagaattagagaattatggagagtgtggcaggtgccctgaagggaagcaacaggatacaagaaaaaatgatggggccaggcacggtggctcacgcctgtaaccccagcaatttggcaggccgaagtgggtggattgcttgagcccaggagttcgagaccagcctgggcaatgtggtgagacccccgtctctacaaaaatgttttaaaaattggttgggcgtggtggcgcatgcctgtatactcagctactagggtggccgacgtgggcttgagcccaggaggtcaaggctgcagtgagctgtgattgtgccactgcactccagcctgggcaacggagagagactctgtctcaaaaataagataaactgaaattaaaaaataggctgggctggccgggcgtggtggctcacgcctgtaatctcagcactttgggaggccgaggcgggtggatcacgaggtcaggagatcgagaccatcttggctaacacggtgaaaccccatctctcctaaaaatacaaaaaattagccaggcgtggtggcgggcgcctgtagtcccagctactcaggaggctgaggcaggagaatggcgtgaacccgggaggcagagtttgcagtgagccgagatcgtgccactgcactccagcctgggcgacagagcgagactctgtctcagaaaaaaaaaaaaaaaaaaaaaaaaataggctggaccgcggccgggcgctgtggctcatgcctgtaatcccagcactttgggagtccaaggccggtgggtcatgagatcaggagttttgagactaggctggccaacacggtgaaaccccgtctctactaaaaatacaagaaaattagctgggtgtggtctcgggtgcctgtaattccagttactggggaagctgaggcaggagaattgcttgaacctgggaggcagagtttgcagtgagccaagatcatgccactacactccagtctgggtgacagagtgagactctgtctcaaaaaaaaaaaaaaaaaaaagggttgggcaaggtggttcacgcctgtaatcccagaactttgggaggctgaggcaggcagatcactggaagtcaggagttcaagaccagcctggccaacatggtgaaaccctgtgtctactaaaaatacaaaatttagccaggcttggtggcgtatgcctgtaatgccagctactcaggaggctgaggcaggagaatcgcttgattgaacctgggaggcagagtttgcagtgggctggggttgtgccactgcactctaggctgggagacagcaagactccatctaaaaaaaaaaaacagaactgggctgggcacagtggcttatatttgtaatcccagcactttgggaggctgaggttggaggactgcttgagcccagagtttgggactacaacagctgaggtaggcggatcacttgaggtcagaagatggagaccagcctggccagcgtggcgaaaccccgtctctaccaaaaatataaaaaattagccaggcgtggtagagggcgcctgtaatctcagctactcaggacgctgaggcaggagaatcgcctgaacctgggaggcggaggttgcagtgagctgagattgcaccactgcactccagcctgggtaacagagcgagactccgtatcaaagaaaaagaaaaaagaaaaaatgctggaggggccactttagataagccctgagttggggctggtttggggggaacatgtaagccaagatcaaaaagcagtgaggggcccgccctgacgactgctgctcacatctgtgtgtcttgcgcaggagacactgtcggacattcgggaaggtgcgccgctaggggtccacctgccttttgtgggctactcctactcctgcatggccctcaggtaagcactgccctggacggcctccaggggccacgaggctgcttgagcttcctgggtcctgctccttggcagccaatggagttgcaggatcagtcttggaaccttactgttttgggcccaaagactcctaagaggccagagttggaggaccttaaattttcagatctatgtacttcaaaatgttagattgaattttaaaacctcagagtcacagactgggcttcccagaatcttgtaaccattaacttttacgtctgtagtacacagagccacaggacttcagaacttggaaaatatgaagtttagacttttacaatcagttgtaaaagaatgcaaattctttgaatcagccatataacaataaggccatttaaaagtattaatttaggcgggccgcggtggctcacgcctgtaatcctagcactttgggaggccaaggcaggtggatcatgaggtcaggagatcgagaccatcctggctaacacggtgaaaccccgtctctactaaaaatacaaaaaaattagccgggcatggtggcgggcgcttgcggtcccagctacttgggaggcgaggcaggagaatggcatgaacccgggaggcggagcttgcagtgagccgagatcatgccactgcactccagcctgggcgacagagcaagactccgtctcaaaaaaaaaaaaaaaaaagtatttatttaggccgggtgtggtggctcacgcctgtaattccagtgctttgggaggatgaggtgggtggatcacctgaggtcaggagttcgagaccagcctgaccaacgtggagaaacctcatctctactaaaaaacaaaattagccaggcgtggtggcatatacctgtaatcccagctactcaggaggctgaggcaggagaatcagaacccaggagggggaggttgtggtgagctgagatcgtgccattgcattccagcctgggcaacaagagtgaaacttcatctcaaaaaaaaaaaaaaaaaagtactaatttacaggctgggcatggtggctcacgcttggaatcccagcactttgggaggctgaagtggacggattgcttcagcccaggagttcaagaccagcctgagcaacataatgagaccctgtctctacaaaaaattgaaaaaatcgtgccaggcatggtggtctgtgcctgcagtcctagctactcaggagtctgaagtaggagaatcacttgagcctggagtttgaggcttcagtgagccatgatagattccagcctaggcaacaaagtgagacctggtctcaacaaaagtattaattacacaaataatgcattgcttatcacaagtaaattagaaaatacagataaggaaaaggaagttgatatctcgtgagctcaccagatggcagtggtccctggctcacacgtgtactgacacatgtttaaatagtggagaacaggtgtttttttggtttgtttttttccccttcctcatgctactttgtctaagagaacagttggttttctagtcagcttttattactggacaacattacacatactataccttatcattaatgaactccagcttgattctgaaccgctgcggggcctgaacggtgggtcaggattgaacccatcctctattagaacccaggcgcatgtccaggatagctaggtcctgagccgtgttcccacaggagggactgctgggttggaggggacagccacttcataccccagggaggagctgtccccttcccacagctgagtggggtgtgctgacctcaagttgccatcttggggtcccatgcccagtcttaggaccacatctgtggaggtggccagagccaagcagtctccccatcaggtcggcctccctgtcctgaggccctgagaagaggggtctgcagcggtcacatgtcaagggaggagatgagctgaccctagaacatgggggtctggaccccaagtccctgcagaaggtttagaaagagcagctcccaggggcccaaggccaggagaggggcagggcttttcctaagcagaggaggggctattggcctacctgggactctgttctcttcgctctgctgctccccttcctcaaatcaggaggtcttggaagcagctgcccctacccacaggccagaagttctggttctccaccagagaatcagcattctgtctccctccccactccctcctcctctccccagggacagtgaggtcccaggccccacacccatggaactggaggccgagcagctgcttgagccacacgtgcaagcgcccagcctggagccctcggtgtccccacaggatgaaacagtaagttggtggaggggagggggtccgtcagggacaattgggagagaaaaggtgagggcttcccgggtggcgtgcactgtagagccctctagggacttcctgaacagaagcagacagaaaccacggagagacgaggttacttcagacatgggacggtctctgtagttacagtggggcattaagtaagggtgtgtgtgttgctggggatctgagaagtcgatctttgagctgagcgctggtgaaggagaaacaagccatggaaggaaaggtgccaagtggtcaggcgagagcctccagggcaaaggccttgggcaggtgggaatcctgatttgttcctgaaaggtagtttggctgaatcattcctgagaaggctggagaggccagcaggaaacaaaacccagcaaggccttttgtcgtgagggcattagggagctggagggattttgagcagcagagggacataggttgtgttagtgtttgagcaccagccctctggtccctgtgtagatttagaggaccagactcagggatggggctgagggaggtagggaagggagggggcttggatcattgcaggagctatggggattccagaaatgttgaggggacggaggagtaggggataaacaaggattcctagcctggaaccagtgcccaagtcctgagtcttccaggagccacaggcagccttaagcctggtccccatacacaggctgaagtggcagttccagcggctgtccctgcggcagaggctgaggccgaggtgacgctgcgggagctccaggaagccctggaggaggaggtgctcacccggcagagcctgagccgggagatggaggccatccgcacggacaaccagaacttcgccaggtcgggatcggggccggggccggggccgggatgcgggccggtggcaacccttggcatcccctctcgtccggcccggacggactcaccgtccttacctccccacagtcaactacgcgaggcagaggctcggaaccgggacctagaggcacacgtccggcagttgcaggagcggatggagttgctgcaggcagagggagccacaggtgagtccctcatgtgtccccttccccggaggaccgggaggaggtgggccgtctgctccgcggggcgtgtatagacacctggaggagggaagggacccacgctggggcacgccgcgccaccgccctccttcgcccctccacgcgccctatgcctctttcttctccttccagctgtcacgggggtccccagtccccgggccacggatccaccttcccatgtaagacccctctctttcccctgcctcagacctgctgcccattctgcagatcccctccctggctcctggtctccccgtccagatatagggctcaccctacgtctttgcgactttagagggcagaagccctttattcagccccagatctccctccgttcaggcctcaccagattccctccgggatctccctagataacctccccaacctcgattcccctcgctgtctctcgccccaccgctgagggctgggctgggctccgatcgggtcacctgtcccttctctctccagctagatggccccccggccgtggctgtgggccagtgcccgctggtggggccaggccccatgcaccgccgccacctgctgctccctgccagggtacgtccggctgcccacgcccccctccgccgtcgcgccccgcgctccacccgccccttgccacccgcttagctgcgcatttgcggggctgggcccacggcaggagggcggatcttcgggcagccaatcaacacaggccgctaggaagcagccaatgacgagttcggacgggattcgaggcgtgcgagtggactaacaacagctgtaggctgttggggcgggggcggggcgcagggaagagtgcgggcccacctatgggcgtaggcggggcgagtcccaggagccaatcagaggcccatgccgggtgttgacctcgccctctccccgcaggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgatccgggcccgccccctagcggccggggagggaggggccgggtccgcggccggcgaacggggctcgaagggtccttgtagccgggaatgctgctgctgctgctgctgctgctgctgctgctgctgctgctgctgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgcccaaagctctggactccacagtgtccgcggtttgcgttgtgggccggaggctccgcagcgggccaatccggaggcgtgtggaggcggccgaaggtctgggaggagctagcgggatgcgaagcggccgaatcagggttgggggaggaaaagccacggggcggggctttggcgtccggccaataggagggcgagcgggccacccggaggcaccgcccccgcccagctgtggcccagctgtgccaccgagcgtcgagaagagggggctgggctggcagcgcgcgcggccatcctccttccactgcgcctgcgcacgccacgcgcatccgctcctgggacgcaagctcgagaaaagttgctgcaaactttctagcccgttccccgcccctcctcccggccagacccgccccccctgcggagccgggaattccgaggggcggagcgcaggccgagatggggaatgtgggggcctgcagaggaccctggagacggaggcgtgcagaagctcagtctcggggcggaggcttcgcgcccttagtcctcctggacggcccgttaccttctgcgttgtcccgatggggaaactgaggccctgagccagaagcacacgctggggggaggcagaaagcgcggccagaggcggagggaaaacaaagggagaatcacagacagacgggagggggacggacacacacaaggggacagagacccgagtggagagctggatctcgccttcccggcgtggggcgcagggtcggccagaaagaagatcgagaagagcggggagtgggggcgaaaaggggggacaggtgggggaggaggctggggaaagcccgagggaggaagagagggagggaggaacttcccaaagttgcaaaacatggctaccttgcctgcggagccgagcgcggggccggcggctgggggggaggcggtggcggcggcggcggcgaccgaagaggaggaggaggaagcgcgccagctcttgcagactttgcaggcggccgagggtgaggcggcggcggcggccggggccggggcgggcgcagcggctgcgggagctgagggcccgggatccccgggcgtccccgggtcgccccccgaggccgcttccgaaccgcccacgggcctccgcttctcgcccgagcaggtggcgtgcgtctgcgaggcgctgctccaggcgggccacgccggccgcttgagccgcttcctgggcgcactgcccccggccgagcgcctacgtggcagcgacccggtgttgcgcgcgcgggccctggtggccttccagcggggcgagtacgccgagctctaccggctactcgagagccgccccttccccgccgcccaccacgccttcctgcaggacctctacctgcgcgcgcgctaccatgaggccgagcgggcccgcggccgcgcgcttggcgcagtggacaagtatcgactgcgcaagaagttcccgctgcccaagaccatctgggacggcgaggagacagtctactgcttcaaggagcgctcccgcgcagcgctcaaggcctgctaccgcggcaaccgctaccccacgccggacgagaagcgccgcctggccacactcaccggcctgtcgctcacgcaggtcagcaactggttcaagaaccggcgacagcgcgaccggaccggggccggaggcggcgcgccctgcaagaggtgaggggcctcgggcggcgcaagtccagctctcccggggacatcccgtccaccagccctcttcccccgtgcccactgctggggccggcgcgccgaggtcctcggacatctcccgggaccagctcacaatctcaggcgcccgcggggcgcggggactaagtgtggacgggacaggcacccgcccgggccctctccccgcacgcgtctcctcttccagcggctccattccgagctccttcccaaatcccatcggtgttggggaatcacactgcggggggcactagagggactgaggaaaaaggacagggcctgtggccactccact

(19) United States
(12) Patent Application Publication
(10) Pub. No.: US 2024/0252666 A1
(43) Pub. Date: Aug. 1, 2024

(54) MUSCLE TARGETING COMPLEXES AND FORMULATIONS FOR TREATING MYOTONIC DYSTROPHY

(71) Applicant: Dyne Therapeutics, Inc., Waltham, MA (US)

(72) Inventors: Scott Hilderbrand, Waltham, MA (US); Timothy Weeden, Waltham, MA (US); John Najim, Waltham, MA (US); Stefano Zanotti, Waltham, MA (US); Romesh R. Subramanian, Framingham, MA (US); Mohammed T. Qatanani, Waltham, MA (US); Cody A, Desjardins, Waltham, MA (US); Kim Tang, Waltham, MA (US); Brendan Quinn, Boston, MA (US)

(73) Assignee: Dyne Therapeutics, Inc,, Waltham, MA (US)

(21) Appl. No.: 18/436,078

(22) Filed: Feb. 8, 2024

Related U.S. Application Data
(62) Division of application No. 18/303,506, filed on Apr. 19, 2023, now Pat. No. 11,931,421.
Application No. 18/303,506 is a continuation of application No. PCT/US2023/065753, filed on Apr. 14, 2023.
(60) Provisional application No. 63/331,727, filed on Apr. 15, 2022.

(51) Int. Cl. A61K 47/68; A61K 47/54; A61P 21/00

(52) U.S. Cl. CPC A61K 47/6807; A61K 47/545; A61K 47/548; A61K 47/6849; A61P 21/00

(57) ABSTRACT

Aspects of the disclosure relate to complexes and other aspects relate to formulations (e.g., aqueous, lyophilized forms) comprising such complexes comprising an oligonucleotide (e.g., useful for targeting DMPK) covalently linked to an antibody (e.g., anti-TfR1 antibody).

### RELATED APPLICATION

This application claims the benefit under 35 U.S.C. § 119(e) of the filing date of U.S. Provisional Application No. 63/331,727, entitled “MUSCLE TARGETING COMPLEXES AND FORMULATIONS FOR TREATING MYOTONIC DYSTROPHY”, filed Apr. 15, 2022; the contents of which are incorporated herein by reference in their entirety.

### FIELD OF THE INVENTION

The present application relates to targeting complexes for delivering oligonucleotide molecular payloads to cells, formulations comprising such complexes, and uses thereof, particularly uses relating to treatment of disease.

### REFERENCE TO AN ELECTRONIC SEQUENCE LISTING

The contents of the electronic sequence listing (D082470076WO00-SEQ-COB.xml; Size: 56,346 bytes; and Date of Creation: Mar. 22, 2023) are herein incorporated by reference in their entirety.

### BACKGROUND

Myotonic dystrophy (DM) is a dominantly inherited genetic disease that is characterized by myotonia, muscle loss or degeneration, diminished muscle function, insulin resistance, cardiac arrhythmia, smooth muscle dysfunction, and neurological abnormalities. DM is the most common form of adult-onset muscular dystrophy, with a worldwide incidence of about 1 in 8000 people worldwide. Two types of the disease, myotonic dystrophy type 1 (DM1) and myotonic dystrophy type 2 (DM2), have been described. DM1, the more common form of the disease, results from a repeat expansion of a CTG trinucleotide repeat in the 3′ non-coding region of DMPK on chromosome 19; DM2 results from a repeat expansion of a CCTG tetranucleotide repeat in the first intron of ZNF9 on chromosome 3. In DM1 patients, the repeat expansion of a CTG trinucleotide repeat, which may comprise greater than about 50 to about 3,000 or more total repeats, leads to generation of toxic RNA repeats capable of forming hairpin structures that bind essential intracellular proteins, e.g. muscleblind-like proteins, with high affinity resulting in protein sequestration and the loss-of-function phenotypes that are characteristic of the disease. Apart from supportive care and treatments to address the symptoms of the disease, no effective therapeutic for DM1 is currently available.

### SUMMARY

According to some aspects, the present disclosure provides complexes and formulations comprising such complexes.

According to some aspects, a formulation provided herein comprises complexes that comprise an oligonucleotide covalently linked to an anti-transferrin receptor 1 (TfR1) antibody, wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16,
wherein the oligonucleotide comprises a 5′-X—Y—Z-3′ configuration, wherein X and Z are flanking regions comprising one or more modified nucleosides and Y is a gap region comprising one or more 2′-deoxyribonucleosides,
and wherein the complexes are formulated with tris(hydroxymethyl)aminomethane and sucrose.

According to some aspects, a formulation provided herein comprises complexes comprising a structure of formula (I): [R¹]ₙ₁—R², wherein each R¹ independently comprises a group of the formula (Ia):

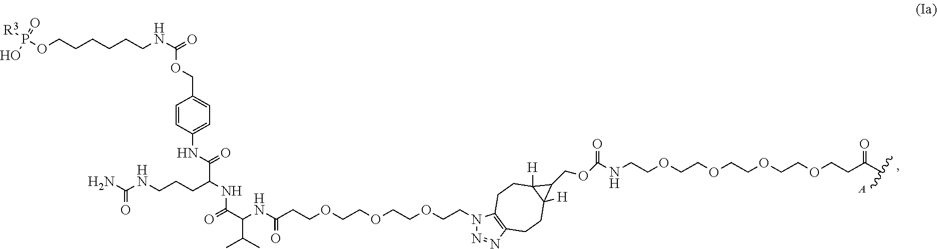

(Ia), wherein
R² comprises an antibody, and
R³ comprises an oligonucleotide comprising a 5′-X—Y—Z-3′ configuration, wherein X and Z are flanking regions comprising one or more modified nucleosides and Y is a gap region comprising one or more 2′-deoxyribonucleosides;
wherein R¹ is covalently linked to R² at attachment point A; and
wherein n1 is an integer representing the number of instances of R¹, wherein each instance of R¹ is covalently linked to a different amino acid residue of the antibody;
wherein the complexes are formulated with tris(hydroxymethyl)aminomethane and sucrose. In some embodiments, each different amino acid residue of the antibody is a lysine. In some embodiments, the antibody is an anti-TfR1 antibody.

In some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16.

In some embodiments, the formulation is in a lyophilized form or a frozen solid form.

In some embodiments, the formulation is in an aqueous solution.

In some embodiments, the tris(hydroxymethyl)aminomethane is present in the aqueous solution at a concentration in the range of 5 mM to 50 mM.

In some embodiments, the sucrose is present in the aqueous solution at a concentration in the range of 5% to 15% weight per volume (w/v %).

In some embodiments, the aqueous solution has a pH in the range of 6.5 to 8.5.

In some embodiments, the tris(hydroxymethyl)aminomethane is present in the aqueous solution at a concentration of 25 mM and/or the sucrose is present in the aqueous solution at a concentration of 10 w/v % and/or the aqueous solution is at a pH of 7.5.

In some embodiments, the antibody is a Fab fragment, a full-length IgG, a Fab′ fragment, a F(ab′)₂ fragment, an scFv, or an Fv.

In some embodiments, the antibody is a Fab fragment.

In some embodiments, the antibody comprises a heavy chain variable region (VH) comprising an amino acid sequence at least 85% identical to SEQ ID NO: 17; and/or wherein the antibody comprises a light chain variable region (VL) comprising an amino acid sequence at least 85% identical to SEQ ID NO: 18. In some embodiments, the antibody comprises a VH comprising the amino acid sequence of SEQ ID NO: 17 and/or a VL comprising the amino acid sequence of SEQ ID NO: 18.

In some embodiments, the antibody comprises a heavy chain comprising an amino acid sequence at least 85% identical to SEQ ID NO: 19; and/or wherein the antibody comprises a light chain comprising an amino acid sequence at least 85% identical to SEQ ID NO: 20. In some embodiments, the antibody comprises a heavy chain comprising the amino acid sequence of SEQ ID NO: 19 and/or a light chain comprising the amino acid sequence of SEQ ID NO: 20.

In some embodiments, the oligonucleotide is 10-30 nucleotides in length. In some embodiments, the oligonucleotide comprises a nucleotide sequence having a region of complementarity of at least 8 consecutive nucleotides in length to SEQ ID NO: 22.

In some embodiments, the oligonucleotide comprises at least 8 consecutive nucleotides of a nucleotide sequence as set forth in SEQ ID NO: 21. In some embodiments, the oligonucleotide comprises the nucleotide sequence of SEQ ID NO: 21.

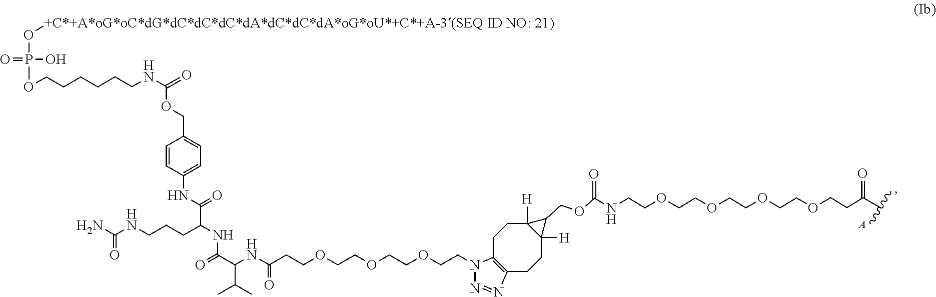

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, such that the oligonucleotide comprises a nucleobase sequence of CAGCGCCCACCAGUCA (SEQ ID NO: 21).

In some embodiments, each R¹ comprises a group of the formula (Ic):

In some embodiments, the complexes are present in the formulation at a concentration in the range of 10 mg/mL to 50 mg/mL.

In some embodiments, the formulation further comprises one or more antibodies that are not covalently linked to an oligonucleotide.

In some embodiments, the average value of n1 of complexes in the formulation is in the range of 0.5 to 5.

According to some aspects, methods of reducing DMPK expression and/or treating myotonic dystrophy in a subject are provided herein, the method comprising administering to the subject an effective amount of a formulation provided herein.

In some embodiments, the subject has an expansion of a disease-associated repeat of a DMPK allele that is associated with myotonic dystrophy. In some embodiments, the disease-associated repeat comprises repeating units of a CTG trinucleotide sequence.

In some embodiments, the complexes reduce DMPK expression in the subject.

### BRIEF DESCRIPTION OF THE DRAWINGS

FIGS. 1A-1D show the amount of DMPK-targeting oligonucleotide (ASO) in the heart (FIG. 1A), diaphragm (FIG. 1B), gastrocnemius (FIG. 1C), or tibialis anterior (FIG. 1D), respectively, after administration of conjugates containing an anti-TfR1 Fab covalently linked to the ASO.

FIGS. 2A-2D show the ability of conjugates containing an anti-TfR1 Fab conjugated to a DMPK-targeting oligonucleotide (ASO) to knock down human DMPK RNA in the heart (FIG. 2A), diaphragm (FIG. 2B), tibialis anterior (FIG. 2C) and gastrocnemius (FIG. 2D) of mice expressing both human TfR1 and two copies of a mutant human DMPK transgene that harbors expanded CTG repeats.

FIGS. 3A-3B show reduced DMPK foci in nuclei of cardiac muscle fibers in mice expressing both human TfR1 and two copies of a mutant human DMPK transgene that harbors expanded CTG repeats and treated with anti-TfR1 Fab conjugated to DMPK-targeting oligonucleotide (ASO). FIG. 3A shows representative images of samples following in situ hybridization staining for DMPK foci and fluorescence staining of myofibers (inset panels). In the microscopy images shown in FIG. 3A, the light rounded shapes show cell nuclei, and the bright puncta within the nuclei show DMPK foci. FIG. 3B shows quantification of DMPK foci.

FIG. 4 shows the splicing correction activity of conjugates containing an anti-TfR1 Fab covalently linked to a DMPK-targeting oligonucleotide (ASO) in the heart of mice expressing both human TfR1 and two copies of a mutant human DMPK transgene that harbors expanded CTG repeats (hTfR1/DMSXL mice). Composite splicing indices based on splicing of Ldb3 exon 11, Mbnl2 exon 6, and Nfix exon 7 are shown for control mice treated with vehicle control (“hTfR1—PBS”), hTfR1/DMSXL mice treated with vehicle control (“hTfR1/DMSXL—PBS”), and hTfR1/DMSXL mice treated with anti-TfR1 Fab-ASO conjugate (“hTfR1/DMSXL—Conjugate”).

FIG. 5 shows the splicing correction activity of conjugates containing an anti-TfR1 Fab covalently linked to a DMPK-targeting oligonucleotide (ASO) in the diaphragm of mice expressing both human TfR1 and two copies of a mutant human DMPK transgene that harbors expanded CTG repeats (hTfR1/DMSXL mice). Composite splicing indices based on splicing of Bin1 exon 11, Insr exon 11, Ldb3 exon 11 and Nfix exon 7 are shown for control mice treated with vehicle control (“hTfR1—PBS”), hTfR1/DMSXL mice treated with vehicle control (“hTfR1/DMSXL—PBS”), and hTfR1/DMSXL mice treated with anti-TfR1 Fab-ASO conjugate (“hTfR1/DMSXL—Conjugate”).

FIG. 6 shows the splicing correction activity of conjugates containing an anti-TfR1 Fab covalently linked to a DMPK-targeting oligonucleotide (ASO) in the tibialis anterior of mice expressing both human TfR1 and two copies of a mutant human DMPK transgene that harbors expanded CTG repeats (hTfR1/DMSXL mice). Composite splicing indices based on splicing of Bin1 exon 11, Ldb3 exon 11, Mbnl2 exon 6, and Nfix exon 7 are shown for control mice treated with vehicle control (“hTfR1—PBS”), hTfR1/DMSXL mice treated with vehicle control (“hTfR1/DMSXL—PBS”), and hTfR1/DMSXL mice treated with anti-TfR1 Fab-ASO conjugate (“hTfR1/DMSXL—Conjugate”).

FIG. 7 shows the splicing correction activity of conjugates containing an anti-TfR1 Fab covalently linked to a DMPK-targeting oligonucleotide (ASO) in the gastrocnemius of mice expressing both human TfR1 and two copies of a mutant human DMPK transgene that harbors expanded CTG repeats (hTfR1/DMSXL mice). Composite splicing indices based on splicing of Mbnl2 exon 6, Nfix exon 7, and Ttn exon 313 are shown for control mice treated with vehicle control (“hTfR1—PBS”), hTfR1/DMSXL mice treated with vehicle control (“hTfR1/DMSXL—PBS”), and hTfR1/DMSXL mice treated with anti-TfR1 Fab-ASO conjugate (“hTfR1/DMSXL—Conjugate”).

FIG. 8 shows DMPK knockdown in DM1 patient myotubes and wild-type non-human primate (NHP) myotubes resulting from incubation with conjugates containing an anti-TfR1 Fab covalently linked to a DMPK-targeting oligonucleotide (ASO). Results are shown normalized to expression in DM1 patient myotubes or NHP myotubes treated with vehicle only. Data are shown as mean+standard deviation for n=4 replicates per condition. Statistics were calculated by one-way ANOVA (*, P<0.05, **, P<0.01).

FIG. 9 shows x-ray diffraction (XRD) pattern of a lyophilized formulation of anti-TfR1 Fab-ASO conjugates.

FIGS. 10A-10C show protein concentrations calculated from absorbance data of a lyophilized formulation of anti-TfR1 Fab-ASO conjugates after reconstitution at 0 weeks (T0), 4 weeks (T4w), and 8 weeks (T8w), at 2-8° C. FIG. 10A shows concentration of the Fab of the conjugates in mg/ml. FIG. 10B shows concentration of the Fab of the conjugates in μM.

FIG. 10C shows concentration of the oligonucleotide of the conjugates in μM.

FIGS. 11A-11B show the average percent recovery (FIG. 11A) and Drug Antibody Ratio (DAR; FIG. 11B) for a lyophilized formulation of anti-TfR1 Fab-ASO conjugates after reconstitution at T0, T4w, and T8w, at 2-8° C.

FIGS. 12A-12C show protein concentrations calculated from absorbance data of a lyophilized formulation of anti-TfR1 Fab-ASO conjugates after reconstitution at 0 weeks (T0), 2 weeks (T2w), 4 weeks (T4w), and 8 weeks (T8w), at 25° C. FIG. 12A shows concentration of the Fab of the conjugates in mg/ml. FIG. 12B shows concentration of the Fab of the conjugates in μM. FIG. 12C shows concentration of the oligonucleotide of the conjugates in μM.

FIGS. 13A-13B show the average percent recovery (FIG. 13A) and Drug Antibody Ratio (DAR; FIG. 13B) for a lyophilized formulation of anti-TfR1 Fab-ASO conjugates after reconstitution at T0, T2w, T4w, and T8w, at 25° C.

FIGS. 14A-14C show protein concentrations calculated from absorbance data of a lyophilized formulation of anti-TfR1 Fab-ASO conjugates after reconstitution at T0, T2w, T4w, and T8w, at 40° C. FIG. 14A shows concentration of the Fab of the conjugates in mg/ml.

FIG. 14B shows concentration of the Fab of the conjugates in μM. FIG. 14C shows concentration of the oligonucleotide of the conjugates in μM.

FIGS. 15A-15B show the average percent recovery (FIG. 15A) and Drug Antibody Ratio (DAR; FIG. 15B) of lyophilized anti-TfR1 Fab-ASO conjugates after reconstitution at T0, T2w, T4w, and T8w, at 40° C.

FIGS. 16A-16B show the sum of the relative area of peaks 1 and 2 (FIG. 16A) and peaks 3 and 4 (FIG. 16B) measured using size-exclusion chromatography (SEC) of lyophilized anti-TfR1 Fab-ASO conjugates after reconstitution at T0, T4w, and T8w, at 2-8° C.

FIGS. 17A-17B show the sum of the relative area of peaks 1 and 2 (FIG. 17A) and peaks 3 and 4 (FIG. 17B) measured using size-exclusion chromatography (SEC) of lyophilized anti-TfR1 Fab-ASO conjugates after reconstitution at T0, T2w, T4w, and T8w, at 25° C.

FIGS. 18A-18B show the sum of the relative area of peaks 1 and 2 (FIG. 18A) and peaks 3 and 4 (FIG. 18B) measured using size-exclusion chromatography (SEC) of lyophilized anti-TfR1 Fab-ASO conjugates after reconstitution at T0, T2w, T4w, T8w, at 40° C.

FIG. 19 shows percent purity of the main peak of lyophilized anti-TfR1 Fab-ASO conjugates after reconstitution measured using capillary electrophoresis (CE-SDS) at T0, T4w, and T8w, at 2-8° C.

FIG. 20 shows percent purity of the main peak of lyophilized anti-TfR1 Fab-ASO conjugates after reconstitution measured using CE-SDS at T0, T2w, T4w, and T8w, at 25° C.

FIG. 21 shows percent purity of the main peak of lyophilized anti-TfR1 Fab-ASO conjugates after reconstitution measured using CE-SDS at T0, T2w, T4w, and T8w, at 40° C.

### DETAILED DESCRIPTION OF INVENTION

According to some aspects, the present disclosure provides complexes and formulations comprising such complexes. In some embodiments, the complexes are formulated with tris(hydroxymethyl)aminomethane and sucrose. In some embodiments, the complexes are formulated as aqueous or lyophilized (e.g., lyophilized powder) forms. In some embodiments, a complex comprises an oligonucleotide (e.g., an oligonucleotide comprising a 5′-X—Y—Z-3′ configuration) covalently linked to an antibody. In some embodiments, a complex comprises a muscle-targeting complex comprising an oligonucleotide (e.g., an oligonucleotide comprising a 5′-X—Y—Z-3′ configuration) covalently linked to an anti-transferrin receptor 1 (TfR1) antibody. In some embodiments, a complex comprises a muscle-targeting complex comprising an oligonucleotide (e.g., an oligonucleotide comprising a 5′-X—Y—Z-3′ configuration) covalently linked to the anti-transferrin receptor 1 (TfR1) antibody provided in Table 2. Also provided are methods of using the complexes and formulations described herein for treating a subject having a myotonic dystrophy (e.g., DM1) and/or methods of reducing the expression or activity of DMPK (e.g., a DMPK RNA) in a cell.

Further aspects of the disclosure, including a description of defined terms, are provided below.

### Definitions

Administering: As used herein, the terms “administering” or “administration” means to provide a complex to a subject in a manner that is physiologically and/or (e.g., and) pharmacologically useful (e.g., to treat a condition in the subject).

Approximately: As used herein, the term “approximately” or “about,” as applied to one or more values of interest, refers to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Antibody: As used herein, the term “antibody” refers to a polypeptide that includes at least one immunoglobulin variable domain or at least one antigenic determinant, e.g., paratope that specifically binds to an antigen. In some embodiments, an antibody is a full-length antibody. In some embodiments, an antibody is a chimeric antibody. In some embodiments, an antibody is a humanized antibody. However, in some embodiments, an antibody is a Fab fragment, a Fab′ fragment, a F(ab′)₂ fragment, a Fv fragment or a scFv fragment. In some embodiments, an antibody is a nanobody derived from a camelid antibody or a nanobody derived from shark antibody. In some embodiments, an antibody is a diabody. In some embodiments, an antibody comprises a framework having a human germline sequence. In another embodiment, an antibody comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody comprises a heavy (H) chain variable region (abbreviated herein as VH), and/or (e.g., and) a light (L) chain variable region (abbreviated herein as VL). In some embodiments, an antibody comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody described herein can be an alpha (α), delta (Δ), epsilon (ε), gamma (γ) or mu (μ) heavy chain. In some embodiments, the heavy chain of an antibody described herein can comprise a human alpha (α), delta (Δ), epsilon (ε), gamma (γ) or mu (μ) heavy chain. In a particular embodiment, an antibody described herein comprises a human gamma 1 CH1, CH2, and/or (e.g., and) CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, the VH domain comprises an amino acid sequence that is at least 70%, 75%, 80%, 85%, 90%, 95%, 98%, or at least 99% identical to any of the variable chain constant regions provided herein. In some embodiments, an antibody is modified, e.g., modified via glycosylation, phosphorylation, sumoylation, and/or (e.g., and) methylation. In some embodiments, an antibody is a glycosylated antibody, which is conjugated to one or more sugar or carbohydrate molecules. In some embodiments, the one or more sugar or carbohydrate molecule are conjugated to the antibody via N-glycosylation, O-glycosylation, C-glycosylation, glypiation (GPI anchor attachment), and/or (e.g., and) phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, an N-acetylgalactosamine unit, a galactose unit, a fucose unit, or a phospholipid unit. In some embodiments, an antibody is a construct that comprises a polypeptide comprising one or more antigen binding fragments of the disclosure linked to a linker polypeptide or an immunoglobulin constant domain. Linker polypeptides comprise two or more amino acid residues joined by peptide bonds and are used to link one or more antigen binding portions. Examples of linker polypeptides have been reported (see e.g., Holliger, P., et al. (1993) Proc. Natl. Acad. Sci. USA 90:6444-6448; Poljak, R. J., et al. (1994) Structure 2:1121-1123). Still further, an antibody may be part of a larger immunoadhesion molecule, formed by covalent or noncovalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

CDR: As used herein, the term “CDR” refers to the complementarity determining region within antibody variable sequences. A typical antibody molecule comprises a heavy chain variable region (VH) and a light chain variable region (VL), which are usually involved in antigen binding. The VH and VL regions can be further subdivided into regions of hypervariability, also known as “complementarity determining regions” (“CDR”), interspersed with regions that are more conserved, which are known as “framework regions” (“FR”). Each VH and VL is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the IMGT definition, the Chothia definition, the AbM definition, and/or (e.g., and) the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242; IMGT®, the international ImMunoGeneTics Information System® http://www.imgt.org, Lefranc, M. P. et al., Nucleic Acids Res., 27:209-212 (1999); Ruiz, M. et al., Nucleic Acids Res., 28:219-221 (2000); Lefranc, M. P., Nucleic Acids Res., 29:207-209 (2001); Lefranc, M. P., Nucleic Acids Res., 31:307-310 (2003); Lefranc, M. P. et al., In Silico Biol., 5, 0006 (2004) [Epub], 5:45-60 (2005); Lefranc, M. P. et al., Nucleic Acids Res., 33:D593-597 (2005); Lefranc, M. P. et al., Nucleic Acids Res., 37:D1006-1012 (2009); Lefranc, M. P. et al., Nucleic Acids Res., 43:D413-422 (2015); Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004). See also hgmp.mrc.ac.uk and bioinf.org.uk/abs. As used herein, a CDR may refer to the CDR defined by any method known in the art. Two antibodies having the same CDR means that the two antibodies have the same amino acid sequence of that CDR as determined by the same method, for example, the IMGT definition.

There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems. Examples of CDR definition systems are provided in Table 1.

TABLE 1
CDR Definitions

| | IMGT¹ | Kabat² | Chothia³ |
|---|---|---|---|
| CDR-H1 | 27-38 | 31-35 | 26-32 |
| CDR-H2 | 56-65 | 50-65 | 53-55 |
| CDR-H3 | 105-116/117 | 95-102 | 96-101 |
| CDR-L1 | 27-38 | 24-34 | 26-32 |
| CDR-L2 | 56-65 | 50-56 | 50-52 |
| CDR-L3 | 105-116/117 | 89-97 | 91-96 |

¹IMGT ®, the international ImMunoGeneTics information system ®, imgt.org, Lefranc, M.-P. et al., Nucleic Acids Res., 27: 209-212 (1999)
²Kabat et al. (1991) Sequences of Proteins of Immunological Interest, Fifth Edition, U.S. Department of Health and Human Services, NIH Publication No. 91-3242
³Chothia et al., J. Mol. Biol. 196: 901-917 (1987))

Complementary: As used herein, the term “complementary” refers to the capacity for precise pairing between two nucleotides or two sets of nucleotides. In particular, complementary is a term that characterizes an extent of hydrogen bond pairing that brings about binding between two nucleotides or two sets of nucleotides. For example, if a base at one position of an oligonucleotide is capable of hydrogen bonding with a base at the corresponding position of a target nucleic acid (e.g., an mRNA), then the bases are considered to be complementary to each other at that position. Base pairings may include both canonical Watson-Crick base pairing and non-Watson-Crick base pairing (e.g., Wobble base pairing and Hoogsteen base pairing). For example, in some embodiments, for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

Covalently linked: As used herein, the term “covalently linked” refers to a characteristic of two or more molecules being linked together via at least one covalent bond. In some embodiments, two molecules can be covalently linked together by a single bond, e.g., a disulfide bond or disulfide bridge, that serves as a linker between the molecules. However, in some embodiments, two or more molecules can be covalently linked together via a molecule that serves as a linker that joins the two or more molecules together through multiple covalent bonds. In some embodiments, a linker may be a cleavable linker. However, in some embodiments, a linker may be a non-cleavable linker.

Disease-associated-repeat: As used herein, the term “disease-associated-repeat” refers to a repeated nucleotide sequence at a genomic location for which the number of units of the repeated nucleotide sequence is correlated with and/or (e.g., and) directly or indirectly contributes to, or causes, genetic disease. Each repeating unit of a disease associated repeat may be 2, 3, 4, 5 or more nucleotides in length. For example, in some embodiments, a disease associated repeat is a dinucleotide repeat. In some embodiments, a disease associated repeat is a trinucleotide repeat. In some embodiments, a disease associated repeat is a tetranucleotide repeat. In some embodiments, a disease associated repeat is a pentanucleotide repeat. In some embodiments, the disease-associated-repeat comprises CAG repeats, CTG repeats, CUG repeats, CGG repeats, CCTG repeats, or a nucleotide complement of any thereof. In some embodiments, a disease-associated-repeat is in a non-coding portion of a gene. However, in some embodiments, a disease-associated-repeat is in a coding region of a gene. In some embodiments, a disease-associated-repeat is expanded from a normal state to a length that directly or indirectly contributes to, or causes, genetic disease. In some embodiments, a disease-associated-repeat is in RNA (e.g., an RNA transcript). In some embodiments, a disease-associated-repeat is in DNA (e.g., a chromosome, a plasmid). In some embodiments, a disease-associated-repeat is expanded in a chromosome of a germline cell. In some embodiments, a disease-associated-repeat is expanded in a chromosome of a somatic cell. In some embodiments, a disease-associated-repeat is expanded to a number of repeating units that is associated with congenital onset of disease. In some embodiments, a disease-associated-repeat is expanded to a number of repeating units that is associated with childhood onset of disease. In some embodiments, a disease-associated-repeat is expanded to a number of repeating units that is associated with adult onset of disease. In DM1, the DMPK gene comprises a disease-associated repeat of CTG units.

DMPK: As used herein, the term “DMPK” refers to a gene that encodes myotonin-protein kinase (also known as myotonic dystrophy protein kinase or dystrophia myotonica protein kinase), a serine/threonine protein kinase. Substrates for this enzyme may include myogenin, the beta-subunit of the L-type calcium channels, and phospholemman. In some embodiments, DMPK may be a human (Gene ID: 1760), non-human primate (e.g., Gene ID: 456139, Gene ID: 715328, Gene ID: 102125829), or rodent gene (e.g., Gene ID: 13400). In humans, a CTG repeat expansion in the Y non-coding, untranslated region of DMPK is associated with myotonic dystrophy type I (DM1). In addition, multiple human transcript variants (e.g., as annotated under GenBank RefSeq Accession Numbers: NM_001081563.2, NM_004409.4, NM_001081560.2, NM_001081562.2, NM_001288764.1, NM_001288765.1, and NM_001288766.1) have been characterized that encode different protein isoforms.

DMPK allele: As used herein, the term “DMPK allele” refers to any one of alternative forms (e.g., wild-type or mutant forms) of a DMPK gene. In some embodiments, a DMPK allele may encode for wild-type myotonin-protein kinase that retains its normal and typical functions. In some embodiments, a DMPK allele may comprise one or more disease-associated-repeat expansions. In some embodiments, normal subjects have two DMPK alleles comprising in the range of 5 to 37 repeat units. In some embodiments, the number of CTG repeat units in subjects having DM1 is in the range of about 50 to about 3,000 or more, with higher numbers of repeats leading to an increased severity of disease. In some embodiments, mildly affected DM1 subjects have at least one DMPK allele having in the range of 50 to 150 repeat units. In some embodiments, subjects with classic DM1 have at least one DMPK allele having in the range of 100 to 1,000 or more repeat units. In some embodiments, subjects having DM1 with congenital onset may have at least one DMPK allele comprising more than 2,000 repeat units.

Framework: As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

Human antibody: The term “human antibody”, as used herein, is intended to include antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

Humanized antibody: The term “humanized antibody” refers to antibodies which comprise heavy and light chain variable region sequences from a non-human species (e.g., a mouse) but in which at least a portion of the VH and/or (e.g., and) VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized anti-transferrin receptor antibodies and antigen binding portions are provided. Such antibodies may be generated by obtaining murine anti-transferrin receptor monoclonal antibodies using traditional hybridoma technology followed by humanization using in vitro genetic engineering, such as those disclosed in Kasaian et al PCT publication No. WO 2005/123126 A2.

Kabat numbering: The terms “Kabat numbering”, “Kabat definitions and “Kabat labeling” are used interchangeably herein. These terms, which are recognized in the art, refer to a system of numbering amino acid residues which are more variable (i.e. hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

Myotonic dystrophy (DM): As used herein, the term “Myotonic dystrophy (DM)” refers to a genetic disease caused by mutations in the DMPK gene or CNBP (ZNF9) gene that is characterized by muscle loss, muscle weakening, and muscle function. Two types of the disease, myotonic dystrophy type 1 (DM1) and myotonic dystrophy type 2 (DM2), have been described. DM1 is associated with an expansion of a CTG trinucleotide repeat in the 3′ non-coding region of DMPK. DM2 is associated with an expansion of a CCTG tetranucleotide repeat in the first intron of ZNF9. In both DM1 and DM2, the nucleotide expansions lead to toxic RNA repeats capable of forming hairpin structures that bind critical intracellular proteins, e.g., muscleblind-like proteins, with high affinity. Myotonic dystrophy, the genetic basis for the disease, and related symptoms are described in the art (see, e.g. Thornton, C. A., “Myotonic Dystrophy” Neurol Clin. (2014), 32(3): 705-719; and Konieczny et al. “Myotonic dystrophy: candidate small molecule therapeutics” Drug Discovery Today (2017), 22:11). In some embodiments, subjects are born with a variation of DM1 called congenital myotonic dystrophy. Symptoms of congenital myotonic dystrophy are present from birth and include weakness of all muscles, breathing problems, clubfeet, developmental delays and intellectual disabilities. DM1 is associated with Online Mendelian Inheritance in Man (OMIM) Entry #160900. DM2 is associated with OMIM Entry #602668.

Oligonucleotide: As used herein, the term “oligonucleotide” refers to an oligomeric nucleic acid compound of up to 200 nucleotides in length. Examples of oligonucleotides include, but are not limited to, RNAi oligonucleotides (e.g., siRNAs, shRNAs), microRNAs, gapmers, mixmers, phosphorodiamidate morpholinos, peptide nucleic acids, aptamers, guide nucleic acids (e.g., Cas9 guide RNAs), etc. Oligonucleotides may be single-stranded or double-stranded. In some embodiments, an oligonucleotide may comprise one or more modified nucleosides (e.g., 2′-O-methyl sugar modifications, purine or pyrimidine modifications). In some embodiments, an oligonucleotide may comprise one or more modified internucleoside linkage. In some embodiments, an oligonucleotide may comprise one or more phosphorothioate linkages, which may be in the Rp or Sp stereochemical conformation.

Region of complementarity: As used herein, the term “region of complementarity” refers to a nucleotide sequence, e.g., of an oligonucleotide, that is sufficiently complementary to a cognate nucleotide sequence, e.g., of a target nucleic acid, such that the two nucleotide sequences are capable of annealing to one another under physiological conditions (e.g., in a cell). In some embodiments, a region of complementarity is fully complementary to a cognate nucleotide sequence of target nucleic acid. However, in some embodiments, a region of complementarity is partially complementary to a cognate nucleotide sequence of target nucleic acid (e.g., at least 80%, 90%, 95% or 99% complementarity). In some embodiments, a region of complementarity contains 1, 2, 3, or 4 mismatches compared with a cognate nucleotide sequence of a target nucleic acid.

Specifically binds: As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less. In some embodiments, an antibody specifically binds to the transferrin receptor, e.g., an epitope of the apical domain of transferrin receptor.

Subject: As used herein, the term “subject” refers to a mammal. In some embodiments, a subject is non-human primate, or rodent. In some embodiments, a subject is a human. In some embodiments, a subject is a patient, e.g., a human patient that has or is suspected of having a disease. In some embodiments, the subject is a human patient who has or is suspected of having a disease resulting from a disease-associated-repeat expansion, e.g., in a DMPK allele.

Transferrin receptor: As used herein, the term, “transferrin receptor” (also known as TFRC, CD71, p90, TFR, or TFR1) refers to an internalizing cell surface receptor that binds transferrin to facilitate iron uptake by endocytosis. In some embodiments, a transferrin receptor may be of human (NCBI Gene ID 7037), non-human primate (e.g., NCBI Gene ID 711568 or NCBI Gene ID 102136007), or rodent (e.g., NCBI Gene ID 22042) origin. In addition, multiple human transcript variants have been characterized that encoded different isoforms of the receptor (e.g., as annotated under GenBank RefSeq Accession Numbers: NP_001121620.1, NP_003225.2, NP_001300894.1, and NP_001300895.1).

2′-modified nucleoside: As used herein, the terms “2′-modified nucleoside” and “2′-modified ribonucleoside” are used interchangeably and refer to a nucleoside having a sugar moiety modified at the 2′ position. In some embodiments, the 2′-modified nucleoside is a 2′-4′ bicyclic nucleoside, where the 2′ and 4′ positions of the sugar are bridged (e.g., via a methylene, an ethylene, or a (S)-constrained ethyl bridge). In some embodiments, the 2′-modified nucleoside is a non-bicyclic 2′-modified nucleoside, e.g., where the 2′ position of the sugar moiety is substituted. Non-limiting examples of 2′-modified nucleosides include: 2′-deoxy, 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA, methylene-bridged nucleic acid), ethylene-bridged nucleic acid (ENA), and (S)-constrained ethyl-bridged nucleic acid (cEt). In some embodiments, the 2′-modified nucleosides described herein are high-affinity modified nucleotides and oligonucleotides comprising the 2′-modified nucleosides have increased affinity to a target sequences, relative to an unmodified oligonucleotide. Examples of structures of 2′-modified nucleosides are provided below:

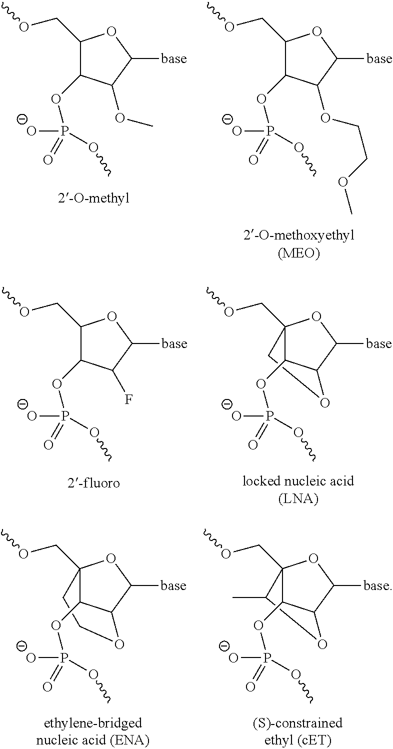

These examples are shown with phosphate groups, but any internucleoside linkages are contemplated between 2′-modified nucleosides.

Ranges: All ranges provided in the present disclosure are inclusive of the end points.

### Complexes

Provided herein are complexes that comprise a targeting agent, e.g., an antibody, covalently linked to an oligonucleotide. In some embodiments, a complex comprises a muscle-targeting antibody covalently linked to one or more oligonucleotides. In some embodiments, the oligonucleotide is an antisense oligonucleotide that targets a DMPK RNA to reduce expression or activity of DMPK (e.g., reduce the level of a mutant or wild-type DMPK RNA, or the activity of a DMPK gene product).

Complexes described herein generally comprise a linker that covalently links an antibody (e.g., an ant-TfR1 antibody) described herein to an oligonucleotide (e.g., an oligonucleotide comprising a 5′-X—Y—Z-3′ configuration). A linker comprises at least one covalent bond.

In some embodiments, complexes described herein comprise a structure of formula (I): [R¹]ₙ₁—R², in which each R¹ independently comprises a compound comprising an oligonucleotide (e.g., an oligonucleotide comprising a 5′-X—Y—Z-3′ configuration) and R² comprises an antibody (e.g., an anti-TfR1 antibody), and wherein in each complex n1 is independently an integer (e.g., one or greater) representing the number of instances of R¹ in each complex. In some embodiments, each R¹ independently comprises a group comprising an oligonucleotide. In some embodiments, each R¹ independently comprises a group that comprises additional elements in addition to an oligonucleotide. In some embodiments, R² comprises an antibody (e.g., an anti-TfR1 antibody) comprising a heavy chain comprising a heavy chain variable region (VH) and a heavy chain constant region, and a light chain comprising a light chain variable region (VL) and a light chain constant region. In some embodiments, each R¹ of a complex is independently covalently linked to a different amino acid residue (e.g., lysine or cysteine) of R².

In some embodiments, in each complex n1 is independently an integer (e.g., one or greater). In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv. In some embodiments, the antibody is a Fab fragment.

In some embodiments, the value of n1 of each or any complex (e.g., any complex in any of the compositions or formulations disclosed herein) is an integer up to the number of amino acid residues in the antibody to which conjugation is desired or targeted (e.g., the number of lysine residues). In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, in each complex the value of n1 is independently selected from 1, 2, 3, 4, 5, 6, 7, 8, 9, 10, 11, 12, 13, 14, 15, 16, 17, 18, 19, 20, 21, 22, 23, 24, 25 and 26. In some embodiments, in each complex the value of n1 is independently in the range of 1-27, 1-26, 1-10, 1-5, or 1-3. In some embodiments, the average value of n1 of complexes of the composition is in the range of 1 to 5 (e.g., 1-5, 1-4, 1-3, 3-5, or 1-2). In some embodiments, compositions described herein comprise complexes that comprise a structure of formula (I): [R¹]ₙ₁—R², wherein n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, in each complex type n1 is independently an integer of one or greater representing the number of instances of R¹ in each complex of the complex type, and in which the different complex types of the composition are characterized by having different n1 values (e.g., n1 values in the range of 1-27, 1-26, 1-25, 1-20, 1-15, 1-10, 1-5, or 1-3).

In some embodiments, compositions are provided (e.g., formulations comprising tris(hydroxymethyl)aminomethane and/or sucrose, as described herein) that comprise a plurality of different complexes. In some embodiments, the plurality of different complexes comprise a common targeting agent (e.g. an antibody) and a common oligonucleotide (e.g., an oligonucleotide comprising a 5′-X—Y—Z-3′ configuration, such as a DMPK-targeting oligonucleotide). In such embodiments, different complex types are characterized by having different numbers of oligonucleotides covalently linked to an antibody. For example, in some embodiments, compositions are provided that comprise a plurality of complexes comprising a structure of formula (I): [R¹]ₙ₁—R², in which each R¹ independently comprises a compound comprising an oligonucleotide (e.g., a DMPK-targeting oligonucleotide) and R² comprises an antibody (e.g., anti-TfR1 antibody), and in which n1 is an integer representing the number of instances of R¹ in a complex, and in which different complexes of the composition may have different n1 values (e.g., n1 values in the range of 1-27, 1-26, 1-10, 1-5, or 1-3). In some embodiments, in complexes of a composition n1 is independently an integer. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, compositions described herein comprise complexes in which n1 is 0.

In some embodiments, a composition described herein comprises antibody that is not conjugated to an oligonucleotide (e.g., in trace amounts) and antibody conjugated to one or more oligonucleotides. In some embodiments, antibody that is not conjugated to an oligonucleotide may be referred to as a compound comprising a structure of formula (I): [R¹]ₙ₁—R², for which n1 is zero. Accordingly, in some embodiments, a composition for administration to a subject in the methods described herein comprises compounds (e.g., complexes) comprising a structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds comprising a structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, each instance of R¹ in a complex is covalently linked to a different amino acid residue of the antibody. In some embodiments, an amino acid to which R¹ is covalently linked comprises an F-amino group (e.g., lysine, arginine). However, in some embodiments, an amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, R¹ is not covalently linked to an amino acid residue residing in a CDR region of the antibody.

In some embodiments, complexes provided herein (e.g., in compositions or formulations described herein) comprise a structure of formula (I): [R¹]ₙ₁—R², in which each instance of R¹ independently comprises a group of the formula (Ia):

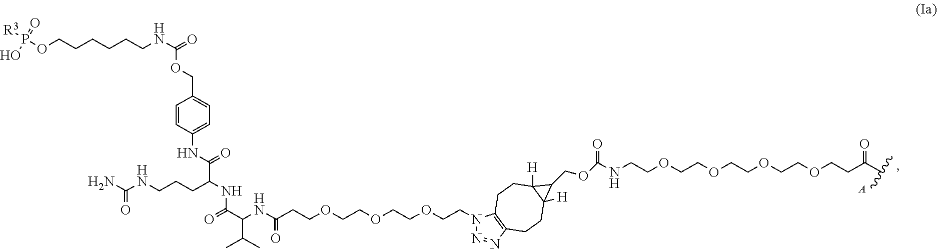

in which R³ comprises an oligonucleotide, e.g., an oligonucleotide comprising a 5′-X—Y—Z-3′ configuration; and R¹ is covalently linked (e.g., indirectly or directly linked, e.g., directly linked) to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R³ comprises an oligonucleotide comprising a nucleobase sequence of CAGCGCCCACCAGUCA (SEQ ID NO: 21). In some embodiments, R³ comprises an oligonucleotide comprising a structure of
+C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage.

In some embodiments, complexes provided herein (e.g., in compositions or formulations described herein) comprise a structure of formula (I): [R¹]ₙ₁—R², in which each R¹ comprises a group of the formula (Ib):

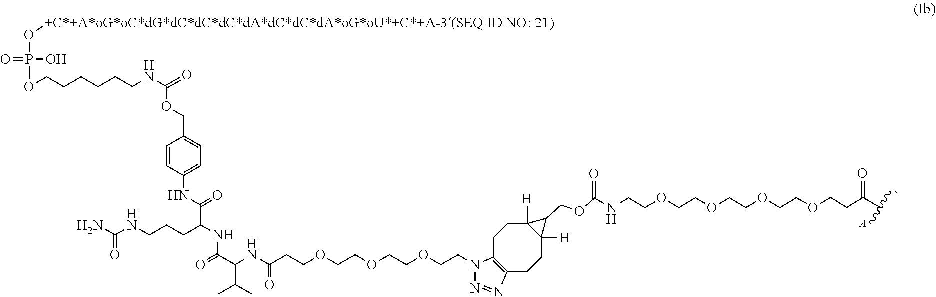

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21), wherein n1 is an integer (e.g., one or greater) representing the number of instances of R¹ in each complex, and each R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv.

In some embodiments, complexes provided herein (e.g., in compositions or formulations described herein) comprise a structure of formula (I): [R¹]ₙ₁— R², in which each R¹ comprises a group of the formula (Ic):

wherein R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv.

In some embodiments, complexes provided herein (e.g., in compositions or formulations described herein) comprise a structure of the formula (Id):

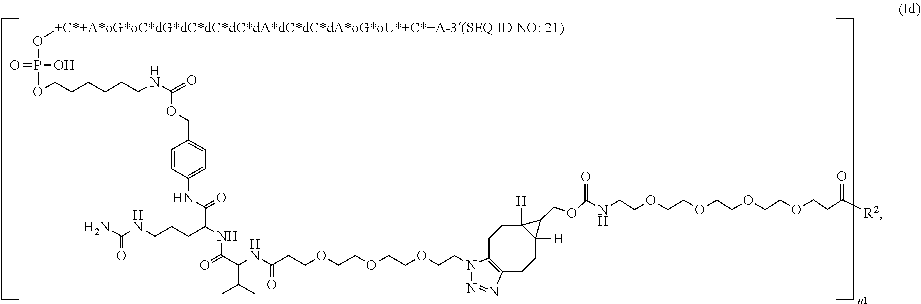

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21); wherein R² comprises an antibody comprising a sequence as set forth in Table 2; wherein n1 is an integer (e.g., one or greater) representing the number of instances of the group enclosed by square brackets, wherein each instance of the group enclosed by square brackets is covalently linked to a different amino acid residue of the antibody, optionally wherein each different amino acid residue is a lysine. In some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv.

In some embodiments, complexes described herein comprise a structure of formula (A):

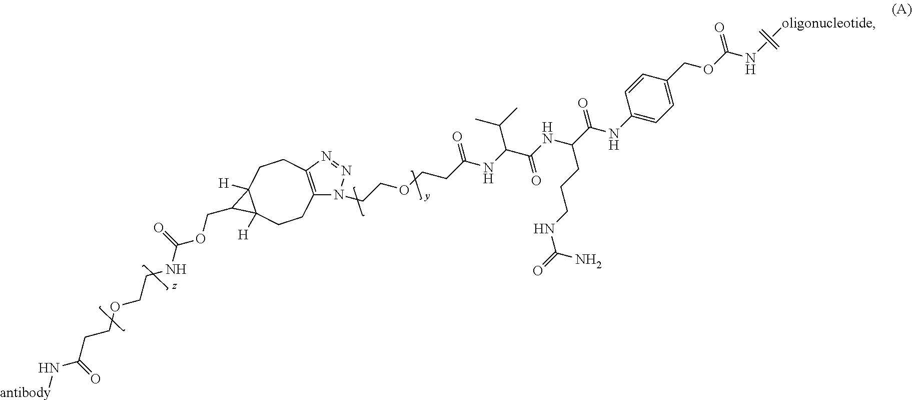

(A), wherein y is 0-15 (e.g., 3) and z is 0-15 (e.g., 4). In some embodiments, the amide shown adjacent the antibody (e.g., anti-TfR1 antibody) in the structure (A) results from a reaction with an amine of the antibody, such as a lysine epsilon amine. In some embodiments, a complex described herein comprises an anti-TfR1 antibody (e.g., an anti-TfR1 Fab) covalently linked via a lysine of the antibody to the 5′ end of an oligonucleotide (e.g., an oligonucleotide comprising a 5′-X—Y—Z-3′ configuration). In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv.

### Antibodies

In some embodiments, complexes described herein comprise an antibody that binds human transferrin receptor 1 (TfR1). An example human TfR1 amino acid sequence, corresponding to NCBI sequence NP_003225.2 (transferrin receptor protein 1 isoform 1, Homo sapiens) is as follows:

(SEQ ID NO: 23)
MMDQARSAFSNLFGGEPLSYTRFSLARQVDGDNSHVEMKLAVDEEENAD
NNTKANVTKPKRCSGSICYGTIAVIVFFLIGFMIGYLGYCKGVEPKTEC
ERLAGTESPVREEPGEDFPAARRLYWDDLKRKLSEKLDSTDFTGTIKLL
NENSYVPREAGSQKDENLALYVENQFREFKLSKVWRDQHFVKIQVKDSA
QNSVIIVDKNGRLVYLVENPGGYVAYSKAATVTGKLVHANFGTKKDFED
LYTPVNGSIVIVRAGKITFAEKVANAESLNAIGVLIYMDQTKFPIVNAE
LSFFGHAHLGTGDPYTPGFPSFNHTQFPPSRSSGLPNIPVQTISRAAAE
KLFGNMEGDCPSDWKTDSTCRMVTSESKNVKLTVSNVLKEIKILNIFGV
IKGFVEPDHYVVVGAQRDAWGPGAAKSGVGTALLLKLAQMFSDMVLKDG
FQPSRSIIFASWSAGDFGSVGATEWLEGYLSSLHLKAFTYINLDKAVLG
TSNFKVSASPLLYTLIEKTMQNVKHPVTGQFLYQDSNWASKVEKLTLDN
AAFPFLAYSGIPAVSFCFCEDTDYPYLGTTMDTYKELIERIPELNKVAR
AAAEVAGQFVIKLTHDVELNLDYERYNSQLLSFVRDLNQYRADIKEMGL
SLOWLYSARGDFFRATSRLTTDFGNAEKTDRFVMKKLNDRVMRVEYHFL
SPYVSPKESPFRHVFWGSGSHTLPALLENLKLRKQNNGAFNETLFRNQL
ALATWTIQGAANALSGDVWDIDNEF.

Table 2 provides examples of sequences of an anti-TfR1 antibody useful in the complexes provided herein.

TABLE 2
Examples of anti-TfR1 antibody sequences

| Feature | IMGT | Kabat | Chothia |
|---|---|---|---|
| CDR-H1 | GYSITSGYY | SGYYWN (SEQ ID | GYSITSGY (SEQ ID NO: 12) |
| | (SEQ ID NO: 1) | NO: 7) | |
| CDR-H2 | ITFDGAN | YITFDGANNYNPSL | FDG (SEQ ID NO: 13) |
| | (SEQ ID NO: 2) | KN (SEQ ID NO: 8) | |
| CDR-H3 | TRSSYDYDVL | SSYDYDVLDY | SYDYDVLD (SEQ ID NO: 14) |
| | DY (SEQ ID NO: 3) | (SEQ ID NO: 9) | |
| CDR-L1 | QDISNF (SEQ | RASQDISNFLN | SQDISNF (SEQ ID NO: 15) |
| | ID NO: 4) | (SEQ ID NO: 10) | |
| CDR-L2 | YTS (SEQ ID | YTSRLHS (SEQ ID | YTS (SEQ ID NO: 5) |
| | NO: 5) | NO: 11) | |
| CDR-L3 | QQGHTLPYT | QQGHTLPYT (SEQ | GHTLPY (SEQ ID NO: 16) |
| | (SEQ ID NO: 6) | ID NO: 6) | |

| | |
|---|---|
| VH | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSS (SEQ ID NO: 17) |
| VL | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIK (SEQ ID NO: 18) |
| Fab HC | QVQLQESGPGLVKPSQTLSLTCTVTGYSITSGYYWNWIRQPPGKGLEWI |
| | GYITFDGANNYNPSLKNRVSISRDTSKNQFSLKLSSVTAEDTATYYCTR |
| | SSYDYDVLDYWGQGTTVTVSSASTKGPSVFPLAPSSKSTSGGTAALGC |
| | LVKDYFPEPVTVSWNSGALTSGVHTFPAVLQSSGLYSLSSVVTVPSSSL |
| | GTQTYICNVNHKPSNTKVDKKVEPKSCDKTHT (SEQ ID NO: 19) |
| Fab LC | DIQMTQSPSSLSASVGDRVTITCRASQDISNFLNWYQQKPGQPVKLLIY |
| | YTSRLHSGVPSRFSGSGSGTDFTLTISSLQPEDFATYYCQQGHTLPYTFG |
| | QGTKLEIKRTVAAPSVFIFPPSDEQLKSGTASVVCLLNNFYPREAKVQW |
| | KVDNALQSGNSQESVTEQDSKDSTYSLSSTLTLSKADYEKHKVYACEV |
| | THQGLSSPVTKSFNRGEC (SEQ ID NO: 20) |

In some embodiments, the anti-TfR1 antibody of the present disclosure comprises a heavy chain complementarity determining region 1 (CDR-H1) of SEQ ID NO: 1 (according to the IMGT definition system), a heavy chain complementarity determining region 2 (CDR-H2) of SEQ ID NO: 2 (according to the IMGT definition system), a heavy chain complementarity determining region 3 (CDR-H3) of SEQ ID NO: 3 (according to the IMGT definition system), a light chain complementarity determining region 1 (CDR-L1) of SEQ ID NO: 4 (according to the IMGT definition system), a light chain complementarity determining region 2 (CDR-L2) of SEQ ID NO: 5 (according to the IMGT definition system), and a light chain complementarity determining region 3 (CDR-L3) of SEQ ID NO: 6 (according to the IMGT definition system).

In some embodiments, the anti-TfR1 antibody of the present disclosure comprises a heavy chain complementarity determining region 1 (CDR-H1) of SEQ ID NO: 7 (according to the Kabat definition system), a heavy chain complementarity determining region 2 (CDR-H2) of SEQ ID NO: 8 (according to the Kabat definition system), a heavy chain complementarity determining region 3 (CDR-H3) of SEQ ID NO: 9 (according to the Kabat definition system), a light chain complementarity determining region 1 (CDR-L1) of SEQ ID NO: 10 (according to the Kabat definition system), a light chain complementarity determining region 2 (CDR-L2) of SEQ ID NO: 11 (according to the Kabat definition system), and a light chain complementarity determining region 3 (CDR-L3) of SEQ ID NO: 6 (according to the Kabat definition system).

In some embodiments, the anti-TfR1 antibody of the present disclosure comprises a heavy chain complementarity determining region 1 (CDR-H1) of SEQ ID NO: 12 (according to the Chothia definition system), a heavy chain complementarity determining region 2 (CDR-H2) of SEQ ID NO: 13 (according to the Chothia definition system), a heavy chain complementarity determining region 3 (CDR-H3) of SEQ ID NO: 14 (according to the Chothia definition system), a light chain complementarity determining region 1 (CDR-L1) of SEQ ID NO: 15 (according to the Chothia definition system), a light chain complementarity determining region 2 (CDR-L2) of SEQ ID NO: 5 (according to the Chothia definition system), and a light chain complementarity determining region 3 (CDR-L3) of SEQ ID NO: 16 (according to the Chothia definition system).

In some embodiments, the anti-TfR1 antibody of the present disclosure comprises a heavy chain variable region (VH) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VH comprising the amino acid sequence of SEQ ID NO: 17. Alternatively or in addition (e.g., in addition), the anti-TfR1 antibody of the present disclosure comprises a light chain variable region (VL) containing no more than 25 amino acid variations (e.g., no more than 25, 24, 23, 22, 21, 20, 19, 18, 17, 16, 15, 14, 13, 12, 11, 10, 9, 8, 7, 6, 5, 4, 3, 2, or 1 amino acid variation) in the framework regions as compared with the VL comprising the amino acid sequence of SEQ ID NO: 18.

In some embodiments, the anti-TfR1 antibody of the present disclosure comprises a VH comprising an amino acid sequence that is at least 75% (e.g., 75%, 80%, 85%, 90%, 95%, 98%, or 99%) identical in the framework regions to the VH comprising the amino acid sequence of SEQ ID NO: 17. Alternatively or in addition (e.g., in addition), in some embodiments, the anti-TfR1 antibody of the present disclosure comprises a VL comprising an amino acid sequence that is at least 75% (e.g., 75%, 80%, 85%, 90%, 95%, 98%, or 99%) identical in the framework regions to the VL comprising the amino acid sequence of SEQ ID NO: 18.

In some embodiments, the anti-TfR1 antibody of the present disclosure comprises a VH comprising the amino acid sequence of SEQ ID NO: 17. Alternatively or in addition (e.g., in addition), in some embodiments, the anti-TfR1 antibody of the present disclosure comprises a VL comprising the amino acid sequence of SEQ ID NO: 18.

In some embodiments, the anti-TfR1 antibody of the present disclosure comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure comprises a light chain comprising an amino acid sequence least 75% (e.g., 75%, 80%, 85%, 90%, 95%, 98%, or 99%) identical to the amino acid sequence of SEQ ID NO: 20. In some embodiments, the anti-TfR1 antibody of the present disclosure is a Fab that comprises a heavy chain comprising an amino acid sequence least 75% (e.g., 75%, 80%, 85%, 90%, 95%, 98%, or 99%) identical to the amino acid sequence of SEQ ID NO: 19. Alternatively or in addition (e.g., in addition), the anti-TfR1 antibody of the present disclosure is a Fab that comprises a light chain comprising an amino acid sequence least 75% (e.g., 75%, 80%, 85%, 90%, 95%, 98%, or 99%) identical to the amino acid sequence of SEQ ID NO: 20.

In some embodiments, the anti-TfR1 antibody of the present disclosure comprises a heavy chain comprising the amino acid sequence of SEQ ID NO: 19. Alternatively or in addition (e.g., in addition), the anti-TfR1 antibody of the present disclosure comprises a light chain comprising the amino acid sequence of SEQ ID NO: 20. In some embodiments, the anti-TfR1 antibody of the present disclosure is a Fab that comprises a heavy chain comprising an amino acid sequence of SEQ ID NO: 19. Alternatively or in addition (e.g., in addition), the anti-TfR1 antibody of the present disclosure is a Fab that comprises a light chain comprising the amino acid sequence of SEQ ID NO: 20.

In some embodiments, the anti-TfR1 antibody provided herein may have one or more post-translational modifications. In some embodiments, N-terminal cyclization, also called pyroglutamate formation (pyro-Glu), may occur in the antibody at N-terminal Glutamate (Glu) and/or Glutamine (Gln) residues during production. As such, it should be appreciated that an antibody specified as having a sequence comprising an N-terminal glutamate or glutamine residue encompasses antibodies that have undergone pyroglutamate formation resulting from a post-translational modification. In some embodiments, pyroglutamate formation occurs in a heavy chain sequence. In some embodiments, pyroglutamate formation occurs in a light chain sequence.

### Oligonucleotides

In some embodiments, an oligonucleotide of the complexes described herein is a single stranded oligonucleotide. In some embodiments, the oligonucleotide is useful for targeting DMPK (e.g., for reducing expression or activity of a DMPK RNA, such as the level of a mutant or wild-type DMPK RNA). In some embodiments, an oligonucleotide that is useful for targeting DMPK RNAs. (e.g., for reducing expression or activity of a DMPK RNA, such as the level of a mutant or wild-type DMPK RNA). In some embodiments, the oligonucleotide comprises a region of complementarity to a DMPK RNA. In some embodiments, the oligonucleotide is useful for reducing levels of toxic DMPK having disease-associated repeat expansions, e.g., in a subject having or suspected of having myotonic dystrophy. In some embodiments, the oligonucleotide is designed to direct RNAse H mediated degradation of the target DMPK RNA residing in the nucleus of cells, e.g., muscle cells (e.g., myotubes) or cells of the nervous system (e.g., central nervous system (CNS) cells). In some embodiments, the oligonucleotide is designed to have desirable bioavailability and/or serum-stability properties. In some embodiments, the oligonucleotide is designed to have desirable binding affinity properties. In some embodiments, the oligonucleotide is designed to have desirable toxicity profiles. In some embodiments, the oligonucleotide is designed to have low-complement activation and/or cytokine induction properties.

In some embodiments, DMPK-targeting oligonucleotides described herein are designed to caused RNase H mediated degradation of DMPK mRNA. It should be appreciated that, in some embodiments, oligonucleotides in one format (e.g., antisense oligonucleotides) may be suitably adapted to another format (e.g., siRNA oligonucleotides) by incorporating functional sequences (e.g., antisense strand sequences) from one format to the other format.

Examples of oligonucleotides useful for targeting DMPK are provided in US Patent Application Publication 20100016215A1, published on Jan. 1, 2010, entitled Compound And Method For Treating Myotonic Dystrophy; US Patent Application Publication 20130237585A1, published Jul. 19, 2010, Modulation Of Dystrophia Myotonica-Protein Kinase (DMPK) Expression; US Patent Application Publication 20150064181A1, published on Mar. 5, 2015, entitled “Antisense Conjugates For Decreasing Expression Of Dmpk”; US Patent Application Publication 20150238627A1, published on Aug. 27, 2015, entitled “Peptide-Linked Morpholino Antisense Oligonucleotides For Treatment Of Myotonic Dystrophy”; and US Patent Application Publication 20160304877A1, published on Oct. 20, 2016, entitled “Compounds And Methods For Modulation Of Dystrophia Myotonica-Protein Kinase (Dmpk) Expression,” the contents of each of which are incorporated herein in their entireties.

In some embodiments, oligonucleotides may comprise a region of complementarity to a sequence set forth as follows, which is an example human DMPK gene sequence (Gene ID 1760; NM_001081560.2):

(SEQ ID NO: 24)
AGGGGGGCTGGACCAAGGGGTGGGGAGAAGGGGAGGAGGCCTCGGCCGG
CCGCAGAGAGAAGTGGCCAGAGAGGCCCAGGGGACAGCCAGGGACAGGC
AGACATGCAGCCAGGGCTCCAGGGCCTGGACAGGGGCTGCCAGGCCCTG
TGACAGGAGGACCCCGAGCCCCCGGCCCGGGGAGGGGCCATGGTGCTGC
CTGTCCAACATGTCAGCCGAGGTGCGGCTGAGGCGGCTCCAGCAGCTGG
TGTTGGACCCGGGCTTCCTGGGGCTGGAGCCCCTGCTCGACCTTCTCCT
GGGCGTCCACCAGGAGCTGGGCGCCTCCGAACTGGCCCAGGACAAGTAC
GTGGCCGACTTCTTGCAGTGGGCGGAGCCCATCGTGGTGAGGCTTAAGG
AGGTCCGACTGCAGAGGGACGACTTCGAGATTCTGAAGGTGATCGGACG
CGGGGCGTTCAGCGAGGTAGCGGTAGTGAAGATGAAGCAGACGGGCCAG
GTGTATGCCATGAAGATCATGAACAAGTGGGACATGCTGAAGAGGGGCG
AGGTGTCGTGCTTCCGTGAGGAGAGGGACGTGTTGGTGAATGGGGACCG
GCGGTGGATCACGCAGCTGCACTTCGCCTTCCAGGATGAGAACTACCTG
TACCTGGTCATGGAGTATTACGTGGGCGGGGACCTGCTGACACTGCTGA
GCAAGTTTGGGGAGCGGATTCCGGCCGAGATGGCGCGCTTCTACCTGGC
GGAGATTGTCATGGCCATAGACTCGGTGCACCGGCTTGGCTACGTGCAC
AGGGACATCAAACCCGACAACATCCTGCTGGACCGCTGTGGCCACATCC
GCCTGGCCGACTTCGGCTCTTGCCTCAAGCTGCGGGCAGATGGAACGGT
GCGGTCGCTGGTGGCTGTGGGCACCCCAGACTACCTGTCCCCCGAGATC
CTGCAGGCTGTGGGCGGTGGGCCTGGGACAGGCAGCTACGGGCCCGAGT
GTGACTGGTGGGCGCTGGGTGTATTCGCCTATGAAATGTTCTATGGGCA
GACGCCCTTCTACGCGGATTCCACGGCGGAGACCTATGGCAAGATCGTC
CACTACAAGGAGCACCTCTCTCTGCCGCTGGTGGACGAAGGGGTCCCTG
AGGAGGCTCGAGACTTCATTCAGCGGTTGCTGTGTCCCCCGGAGACACG
GCTGGGCCGGGGTGGAGCAGGCGACTTCCGGACACATCCCTTCTTCTTT
GGCCTCGACTGGGATGGTCTCCGGGACAGCGTGCCCCCCTTTACACCGG
ATTTCGAAGGTGCCACCGACACATGCAACTTCGACTTGGTGGAGGACGG
GCTCACTGCCATGGAGACACTGTCGGACATTCGGGAAGGTGCGCCGCTA
GGGGTCCACCTGCCTTTTGTGGGCTACTCCTACTCCTGCATGGCCCTCA
GGGACAGTGAGGTCCCAGGCCCCACACCCATGGAACTGGAGGCCGAGCA
GCTGCTTGAGCCACACGTGCAAGCGCCCAGCCTGGAGCCCTCGGTGTCC
CCACAGGATGAAACAGCTGAAGTGGCAGTTCCAGCGGCTGTCCCTGCGG
CAGAGGCTGAGGCCGAGGTGACGCTGCGGGAGCTCCAGGAAGCCCTGGA
GGAGGAGGTGCTCACCCGGCAGAGCCTGAGCCGGGAGATGGAGGCCATC
CGCACGGACAACCAGAACTTCGCCAGTCAACTACGCGAGGCAGAGGCTC
GGAACCGGGACCTAGAGGCACACGTCCGGCAGTTGCAGGAGCGGATGGA
GTTGCTGCAGGCAGAGGGAGCCACAGCTGTCACGGGGGTCCCCAGTCCC
CGGGCCACGGATCCACCTTCCCATCTAGATGGCCCCCCGGCCGTGGCTG
TGGGCCAGTGCCCGCTGGTGGGGCCAGGCCCCATGCACCGCCGCCACCT
GCTGCTCCCTGCCAGGGTCCCTAGGCCTGGCCTATCGGAGGCGCTTTCC
CTGCTCCTGTTCGCCGTTGTTCTGTCTCGTGCCGCCGCCCTGGGCTGCA
TTGGGTTGGTGGCCCACGCCGGCCAACTCACCGCAGTCTGGCGCCGCCC
AGGAGCCGCCCGCGCTCCCTGAACCCTAGAACTGTCTTCGACTCCGGGG
CCCCGTTGGAAGACTGAGTGCCCGGGGCACGGCACAGAAGCCGCGCCCA
CCGCCTGCCAGTTCACAACCGCTCCGAGCGTGGGTCTCCGCCCAGCTCC
AGTCCTGTGATCCGGGCCCGCCCCCTAGCGGCCGGGGAGGGAGGGGCCG
GGTCCGCGGCCGGCGAACGGGGCTCGAAGGGTCCTTGTAGCCGGGAATG
CTGCTGCTGCTGCTGCTGCTGCTGCTGCTGCTGCTGCTGCTGCTGCTGC
TGCTGCTGCTGGGGGGATCACAGACCATTTCTTTCTTTCGGCCAGGCTG
AGGCCCTGACGTGGATGGGCAAACTGCAGGCCTGGGAAGGCAGCAAGCC
GGGCCGTCCGTGTTCCATCCTCCACGCACCCCCACCTATCGTTGGTTCG
CAAAGTGCAAAGCTTTCTTGTGCATGACGCCCTGCTCTGGGGAGCGTCT
GGCGCGATCTCTGCCTGCTTACTCGGGAAATTTGCTTTTGCCAAACCCG
CTTTTTCGGGGATCCCGCGCCCCCCTCCTCACTTGCGCTGCTCTCGGAG
CCCCAGCCGGCTCCGCCCGCTTCGGCGGTTTGGATATTTATTGACCTCG
TCCTCCGACTCGCTGACAGGCTACAGGACCCCCAACAACCCCAATCCAC
GTTTTGGATGCACTGAGACCCCGACATTCCTCGGTATTTATTGTCTGTC
CCCACCTAGGACCCCCACCCCCGACCCTCGCGAATAAAAGGCCCTCCAT
CTGCCCAAAGCTCTGGA.

In some embodiments, oligonucleotides may comprise a region of complementarity to a sequence set forth as follows, which is an example mouse DMPK gene sequence (Gene ID 13400; NM_001190490.1).

(SEQ ID NO: 25)
GAACTGGCCAGAGAGACCCAAGGGATAGTCAGGGACGGGCAGACATGCA
GCTAGGGTTCTGGGGCCTGGACAGGGGCAGCCAGGCCCTGTGACGGGAA
GACCCCGAGCTCCGGCCCGGGGAGGGGCCATGGTGTTGCCTGCCCAACA
TGTCAGCCGAAGTGCGGCTGAGGCAGCTCCAGCAGCTGGTGCTGGACCC
AGGCTTCCTGGGACTGGAGCCCCTGCTCGACCTTCTCCTGGGCGTCCAC
CAGGAGCTGGGTGCCTCTCACCTAGCCCAGGACAAGTATGTGGCCGACT
TCTTGCAGTGGGTGGAGCCCATTGCAGCAAGGCTTAAGGAGGTCCGACT
GCAGAGGGATGATTTTGAGATTTTGAAGGTGATCGGGCGTGGGGCGTTC
AGCGAGGTAGCGGTGGTGAAGATGAAACAGACGGGCCAAGTGTATGCCA
TGAAGATTATGAATAAGTGGGACATGCTGAAGAGAGGCGAGGTGTCGTG
CTTCCGGGAAGAAAGGGATGTATTAGTGAAAGGGGACCGGCGCTGGATC
ACACAGCTGCACTTTGCCTTCCAGGATGAGAACTACCTGTACCTGGTCA
TGGAATACTACGTGGGCGGGGACCTGCTAACGCTGCTGAGCAAGTTTGG
GGAGCGGATCCCCGCCGAGATGGCTCGCTTCTACCTGGCCGAGATTGTC
ATGGCCATAGACTCCGTGCACCGGCTGGGCTACGTGCACAGGGACATCA
AACCAGATAACATTCTGCTGGACCGATGTGGGCACATTCGCCTGGCAGA
CTTCGGCTCCTGCCTCAAACTGCAGCCTGATGGAATGGTGAGGTCGCTG
GTGGCTGTGGGCACCCCGGACTACCTGTCTCCTGAGATTCTGCAGGCCG
TTGGTGGAGGGCCTGGGGCAGGCAGCTACGGGCCAGAGTGTGACTGGTG
GGCACTGGGCGTGTTCGCCTATGAGATGTTCTATGGGCAGACCCCCTTC
TACGCGGACTCCACAGCCGAGACATATGCCAAGATTGTGCACTACAGGG
AACACTTGTCGCTGCCGCTGGCAGACACAGTTGTCCCCGAGGAAGCTCA
GGACCTCATTCGTGGGCTGCTGTGTCCTGCTGAGATAAGGCTAGGTCGA
GGTGGGGCAGACTTCGAGGGTGCCACGGACACATGCAATTTCGATGTGG
TGGAGGACCGGCTCACTGCCATGGTGAGCGGGGGCGGGGAGACGCTGTC
AGACATGCAGGAAGACATGCCCCTTGGGGTGCGCCTGCCCTTCGTGGGC
TACTCCTACTGCTGCATGGCCTTCAGAGACAATCAGGTCCCGGACCCCA
CCCCTATGGAACTAGAGGCCCTGCAGTTGCCTGTGTCAGACTTGCAAGG
GCTTGACTTGCAGCCCCCAGTGTCCCCACCGGATCAAGTGGCTGAAGAG
GCTGACCTAGTGGCTGTCCCTGCCCCTGTGGCTGAGGCAGAGACCACGG
TAACGCTGCAGCAGCTCCAGGAAGCCCTGGAAGAAGAGGTTCTCACCCG
GCAGAGCCTGAGCCGCGAGCTGGAGGCCATCCGGACCGCCAACCAGAAC
TTCTCCAGCCAACTACAGGAGGCCGAGGTCCGAAACCGAGACCTGGAGG
CGCATGTTCGGCAGCTACAGGAACGGATGGAGATGCTGCAGGCCCCAGG
AGCCGCAGCCATCACGGGGGTCCCCAGTCCCCGGGCCACGGATCCACCT
TCCCATCTAGATGGCCCCCCGGCCGTGGCTGTGGGCCAGTGCCCGCTGG
TGGGGCCAGGCCCCATGCACCGCCGTCACCTGCTGCTCCCTGCCAGGAT
CCCTAGGCCTGGCCTATCCGAGGCGCGTTGCCTGCTCCTGTTCGCCGCT
GCTCTGGCTGCTGCCGCCACACTGGGCTGCACTGGGTTGGTGGCCTATA
CCGGCGGTCTCACCCCAGTCTGGTGTTTCCCGGGAGCCACCTTCGCCCC
CTGAACCCTAAGACTCCAAGCCATCTTTCATTTAGGCCTCCTAGGAAGG
TCGAGCGACCAGGGAGCGACCCAAAGCGTCTCTGTGCCCATCGCGCCCC
CCCCCCCCCCCCACCGCTCCGCTCCACACTTCTGTGAGCCTGGGTCCCC
ACCCAGCTCCGCTCCTGTGATCCAGGCCTGCCACCTGGCGGCCGGGGAG
GGAGGAACAGGGCTCGTGCCCAGCACCCCTGGTTCCTGCAGAGCTGGTA
GCCACCGCTGCTGCAGCAGCTGGGCATTCGCCGACCTTGCTTTACTCAG
CCCCGACGTGGATGGGCAAACTGCTCAGCTCATCCGATTTCACTTTTTC
ACTCTCCCAGCCATCAGTTACAAGCCATAAGCATGAGCCCCCTATTTCC
AGGGACATCCCATTCCCATAGTGATGGATCAGCAAGACCTCTGCCAGCA
CACACGGAGTCTTTGGCTTCGGACAGCCTCACTCCTGGGGGTTGCTGCA
ACTCCTTCCCCGTGTACACGTCTGCACTCTAACAACGGAGCCACAGCTG
CACTCCCCCCTCCCCCAAAGCAGTGTGGGTATTTATTGATCTTGTTATC
TGACTCACTGACAGACTCCGGGACCCACGTTTTAGATGCATTGAGACTC
GACATTCCTCGGTATTTATTGTCTGTCCCCACCTACGACCTCCACTCCC
GACCCTTGCGAATAAAATACTTCTGGTCTGCCCTAAA.

In some embodiments, an oligonucleotide may comprise a region of complementarity to DMPK gene sequences of multiple species, e.g., selected from human, mouse and non-human species (e.g., cynomolgus monkey).

In some embodiments, the oligonucleotide may comprise a region of complementarity to a mutant form of DMPK, for example, a mutant form as reported in Botta A. et al. “The CTG repeat expansion size correlates with the splicing defects observed in muscles from myotonic dystrophy type 1 patients.” J Med Genet. 2008 October; 45(10):639-46; and Machuca-Tzili L. et al. “Clinical and molecular aspects of the myotonic dystrophies: a review.” Muscle Nerve. 2005 July; 32(1):1-18; the contents of each of which are incorporated herein by reference in their entireties.

In some embodiments, an oligonucleotide provided herein is an antisense oligonucleotide targeting DMPK. In some embodiments, the oligonucleotide targeting DMPK is any one of the antisense oligonucleotides targeting DMPK as described in US Patent Application Publication US20160304877A1, published on Oct. 20, 2016, entitled “Compounds And Methods For Modulation Of Dystrophia Myotonica-Protein Kinase (DMPK) Expression,” incorporated herein by reference). In some embodiments, the DMPK targeting oligonucleotide targets a region of the DMPK gene sequence as set forth in Genbank accession No. NM_001081560.2 (SEQ ID NO: 24) or as set forth in Genbank accession No. NG_009784.1 (SEQ ID NO: 26).

In some embodiments, a DMPK targeting oligonucleotide provided herein comprises a nucleotide sequence comprising a region complementary to a target region that is at least 8 continuous nucleotides (e.g., at least 8, at least 9, at least 10, at least 12, at least 14, at least 16, at least 18, at least 20 or more continuous nucleotides) of SEQ ID NO: 24.

In some embodiments, a DMPK targeting oligonucleotide provided herein is 10-35 (e.g., 10-35, 10-30, 10-25, 10-20, 10-15, 15-35, 15-30, 15-25, 15-20, 20-35, 20-30, 13-18, 14-17, 15-18, 20-30, 15-17, 27-30, 25-35, or 30-35) nucleotides in length. In some embodiments, a DMPK targeting oligonucleotide provided herein is 10, 11, 12, 13, 14, 15, 16, 17, 18, 19, 20, 21, 22, 23, 24, 25, 26, 27, 28, 29, or 30 nucleotides in length, optionally 15-30, or 16 nucleotides in length. In some embodiments, a DMPK targeting oligonucleotide provided herein is 16 nucleotides in length.

In some embodiments, a DMPK targeting oligonucleotide provided herein comprises a region of complementarity of at least 8 (e.g., at least 8, 9, 10, 11, 12, 13, 14, 15, 16, 17, 18, 19, 20, 21, 22, 23, 24, 25, 26, 27, 28, 29, 30, or more) consecutive nucleotides to a DMPK RNA.

In some embodiments, a DMPK targeting oligonucleotide provided herein comprises a region of complementarity of at least 8 (e.g., at least 8, 9, 10, 11, 12, 13, 14, 15, 16, 17, 18, 19, 20, 21, 22, 23, 24, 25, 26, 27, 28, 29, 30, or more) consecutive nucleotides to a DMPK sequence as set forth in SEQ ID NO: 24 or 25.

In some embodiments, a DMPK targeting oligonucleotide provided herein comprises a region of complementarity of at least 8 (e.g., at least 8, 9, 10, 11, 12, 13, 14, 15, or 16) consecutive nucleotides to a target sequence as set forth in SEQ ID NO: 22 (TGACTGGTGGGCGCTG). In some embodiments, an oligonucleotide useful for targeting DMPK comprises at least 8 (e.g., at least 8, 9, 10, 11, 12, 13, 14, 15, or 16) consecutive nucleotides of a sequence as set forth in SEQ ID NO: 21 (CAGCGCCCACCAGUCA). In some embodiments, an oligonucleotide useful for targeting DMPK comprises the nucleotide sequence of SEQ ID NO: 21.

In some embodiments, the DMPK targeting oligonucleotide comprises a 5′-X—Y—Z-3′ configuration. An oligonucleotide comprising a 5′-X—Y—Z-3′ configuration can refer to a chimeric antisense compound in which a gap region having a plurality of nucleosides that support RNase H cleavage is positioned between flanking regions having one or more nucleosides, wherein the nucleosides comprising the gap region are chemically distinct from the nucleoside or nucleosides comprising the flanking regions. In some embodiments, an oligonucleotide described herein (e.g., a DMPK-targeting oligonucleotide described herein) comprises a 5′-X—Y—Z-3′ configuration, with X and Z as flanking regions around a gap region Y. In some embodiments, the gap region Y comprises one or more 2′-deoxyribonucleosides. In some embodiments, each nucleoside in the gap region Y is a 2′-deoxyribonucleoside, and neither the flanking region X nor the flanking region Z contains any 2′-deoxyribonucleosides.

In some embodiments, the gap region Y comprises a continuous stretch of 6 or more 2′-deoxyribonucleosides, which are capable of recruiting an RNAse, such as RNAse H. In some embodiments, the oligonucleotide binds to the target nucleic acid, at which point an RNAse is recruited and can then cleave the target nucleic acid. In some embodiments, the flanking regions X and Z each comprise one or more modified nucleosides. In some embodiments, flanking regions X and Z each comprise one or more high-affinity modified nucleosides, e.g., one to six high-affinity modified nucleosides. Examples of high affinity modified nucleosides include, but are not limited to, 2^{T}-modified nucleosides (e.g., 2′-MOE, 2′-O-Me, 2′-F) or 2′-4′ bicyclic nucleosides (e.g., LNA, cEt, ENA). In some embodiments, the flanking regions X and Z may be of 1-20 nucleotides, 1-8 nucleotides, or 1-5 nucleotides in length. The flanking regions X and Z may be of similar length or of dissimilar lengths. In some embodiments, the gap region Y may comprise a nucleotide sequence of 5-20 nucleotides, 5-15 nucleotides, 5-12 nucleotides, or 6-10 nucleotides in length.

In some embodiments, the gap region Y comprises one or more unmodified internucleoside linkages. In some embodiments, one or both flanking regions X and Z each independently comprise phosphorothioate internucleoside linkages (e.g., phosphorothioate internucleoside linkages or other linkages) between at least two, at least three, at least four, at least five or more nucleotides. In some embodiments, the gap region Y and two flanking regions X and Z each independently comprise modified internucleoside linkages (e.g., phosphorothioate internucleoside linkages or other linkages) between at least two, at least three, at least four, at least five or more nucleotides.

In some embodiments, the gap region Y in the gapmer is 5-20 nucleosides in length. For example, the gap region Y may be 5-20, 5-15, 5-10, 10-20, 10-15, or 15-20 nucleosides in length. In some embodiments, the gap region Y is 5, 6, 7, 8, 9, 10, 11, 12, 13, 14, 15, 16, 17, 18, 19, or 20 nucleosides in length. In some embodiments, each nucleoside in the gap region Y is a 2′-deoxyribonucleoside. In some embodiments, all nucleosides in the gap region Y are 2′-deoxyribonucleosides. In some embodiments, one or more of the nucleosides in the gap region Y is a modified nucleoside (e.g., a 2′ modified nucleoside such as those described herein). In some embodiments, one or more cytosines in the gap region Y are optionally 5-methyl-cytosines. In some embodiments, each cytosine in the gap region Y is a 5-methyl-cytosine.

In some embodiments, the flanking region X of the oligonucleotide (X in the 5′-X—Y—Z-3′ configuration) and the flanking region Z of the oligonucleotide (Z in the 5′-X—Y—Z-3′ configuration) are independently 1-20 nucleosides long. For example, the flanking region X of the oligonucleotide and the flanking region Z of the oligonucleotide may be independently 1-20, 1-15, 1-10, 1-7, 1-5, 1-3, 1-2, 2-5, 2-7, 3-5, 3-7, 5-20, 5-15, 5-10, 10-20, 10-15, or 15-20 nucleosides long. In some embodiments, the flanking region X of the oligonucleotide and the flanking region Z of the oligonucleotide are independently 1, 2, 3, 4, 5, 6, 7, 8, 9, 10, 11, 12, 13, 14, 15, 16, 17, 18, 19, or 20 nucleosides long. In some embodiments, the flanking region X of the oligonucleotide and the flanking region Z of the oligonucleotide are of the same length. In some embodiments, the flanking region X of the oligonucleotide and the flanking region Z of the oligonucleotide are of different lengths. In some embodiments, the flanking region X of the oligonucleotide is longer than the flanking region Z of the oligonucleotide. In some embodiments, the flanking region X of the oligonucleotide is shorter than the flanking region Z of the oligonucleotide.

In some embodiments, an oligonucleotide described herein (e.g., a DMPK targeting oligonucleotide) comprises a 5′-X—Y—Z-3′ configuration of 5-10-5, 4-12-4, 3-14-3, 2-16-2, 1-18-1, 3-10-3, 2-10-2, 1-10-1, 2-8-2, 4-6-4, 3-6-3, 2-6-2, 4-7-4, 3-7-3, 2-7-2, 4-8-4, 3-8-3, 2-8-2, 1-8-1, 2-9-2, 1-9-1, 2-10-2, 1-10-1, 1-12-1, 1-16-1, 2-15-1, 1-15-2, 1-14-3, 3-14-1, 2-14-2, 1-13-4, 4-13-1, 2-13-3, 3-13-2, 1-12-5, 5-12-1, 2-12-4, 4-12-2, 3-12-3, 1-11-6, 6-11-1, 2-11-5, 5-11-2, 3-11-4, 4-11-3, 1-17-1, 2-16-1, 1-16-2, 1-15-3, 3-15-1, 2-15-2, 1-14-4, 4-14-1, 2-14-3, 3-14-2, 1-13-5, 5-13-1, 2-13-4, 4-13-2, 3-13-3, 1-12-6, 6-12-1, 2-12-5, 5-12-2, 3-12-4, 4-12-3, 1-11-7, 7-11-1, 2-11-6, 6-11-2, 3-11-5, 5-11-3, 4-11-4, 1-18-1, 1-17-2, 2-17-1, 1-16-3, 1-16-3, 2-16-2, 1-15-4, 4-15-1, 2-15-3, 3-15-2, 1-14-5, 5-14-1, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-18-1, 1-17-2, 2-17-1, 1-16-3, 3-16-1, 2-16-2, 1-15-4, 4-15-1, 2-15-3, 3-15-2, 1-14-5, 2-14-4, 4-14-2, 3-14-3, 1-13-6, 6-13-1, 2-13-5, 5-13-2, 3-13-4, 4-13-3, 1-12-7, 7-12-1, 2-12-6, 6-12-2, 3-12-5, 5-12-3, 1-11-8, 8-11-1, 2-11-7, 7-11-2, 3-11-6, 6-11-3, 4-11-5, 5-11-4, 1-19-1, 1-18-2, 2-18-1, 1-17-3, 3-17-1, 2-17-2, 1-16-4, 4-16-1, 2-16-3, 3-16-2, 1-15-5, 2-15-4, 4-15-2, 3-15-3, 1-14-6, 6-14-1, 2-14-5, 5-14-2, 3-14-4, 4-14-3, 1-13-7, 7-13-1, 2-13-6, 6-13-2, 3-13-5, 5-13-3, 4-13-4, 1-12-8, 8-12-1, 2-12-7, 7-12-2, 3-12-6, 6-12-3, 4-12-5, 5-12-4, 2-11-8, 8-11-2, 3-11-7, 7-11-3, 4-11-6, 6-11-4, 5-11-5, 1-20-1, 1-19-2, 2-19-1, 1-18-3, 3-18-1, 2-18-2, 1-17-4, 4-17-1, 2-17-3, 3-17-2, 1-16-5, 2-16-4, 4-16-2, 3-16-3, 1-15-6, 6-15-1, 2-15-5, 5-15-2, 3-15-4, 4-15-3, 1-14-7, 7-14-1, 2-14-6, 6-14-2, 3-14-5, 5-14-3, 4-14-4, 1-13-8, 8-13-1, 2-13-7, 7-13-2, 3-13-6, 6-13-3, 4-13-5, 5-13-4, 2-12-8, 8-12-2, 3-12-7, 7-12-3, 4-12-6, 6-12-4, 5-12-5, 3-11-8, 8-11-3, 4-11-7, 7-11-4, 5-11-6, 6-11-5, 1-21-1, 1-20-2, 2-20-1, 1-20-3, 3-19-1, 2-19-2, 1-18-4, 4-18-1, 2-18-3, 3-18-2, 1-17-5, 2-17-4, 4-17-2, 3-17-3, 1-16-6, 6-16-1, 2-16-5, 5-16-2, 3-16-4, 4-16-3, 1-15-7, 7-15-1, 2-15-6, 6-15-2, 3-15-5, 5-15-3, 4-15-4, 1-14-8, 8-14-1, 2-14-7, 7-14-2, 3-14-6, 6-14-3, 4-14-5, 5-14-4, 2-13-8, 8-13-2, 3-13-7, 7-13-3, 4-13-6, 6-13-4, 5-13-5, 1-12-10, 10-12-1, 2-12-9, 9-12-2, 3-12-8, 8-12-3, 4-12- 7, 7-12-4, 5-12-6, 6-12-5, 4-11-8, 8-11-4, 5-11-7, 7-11-5, 6-11-6, 1-22-1, 1-21-2, 2-21-1, 1-21-3, 3-20-1, 2-20-2, 1-19-4, 4-19-1, 2-19-3, 3-19-2, 1-18-5, 2-18-4, 4-18-2, 3-18-3, 1-17-6, 6-17-1, 2-17-5, 5-17-2, 3-17-4, 4-17-3, 1-16-7, 7-16-1, 2-16-6, 6-16-2, 3-16-5, 5-16-3, 4-16-4, 1-15-8, 8-15-1, 2-15-7, 7-15-2, 3-15-6, 6-15-3, 4-15-5, 5-15-4, 2-14-8, 8-14-2, 3-14-7, 7-14-3, 4-14-6, 6-14-4, 5-14-5, 3-13-8, 8-13-3, 4-13-7, 7-13-4, 5-13-6, 6-13-5, 4-12-8, 8-12-4, 5-12-7, 7-12-5, 6-12-6, 5-11-8, 8-11-5, 6-11-7, or 7-11-6. The numbers indicate the number of nucleosides in X, Y, and Z regions, respectively, in an oligonucleotide comprising the 5′-X—Y—Z-3′ configuration.

In some embodiments, one or more nucleosides in the flanking region X of the oligonucleotide (X in the 5′-X—Y—Z-3′ configuration) or the flanking region Z of the oligonucleotide (Z in the 5′-X—Y—Z-3′ configuration) are modified nucleosides (e.g., high-affinity modified nucleosides). In some embodiments, the modified nucleoside (e.g., high-affinity modified nucleosides) is a 2′-modified nucleoside. In some embodiments, the 2′-modified nucleoside is a 2′-4′ bicyclic nucleoside or a non-bicyclic 2′-modified nucleoside. In some embodiments, the high-affinity modified nucleoside is a 2′-4′ bicyclic nucleoside (e.g., LNA, cEt, or ENA) or a non-bicyclic 2′-modified nucleoside (e.g., 2′-fluoro (2′-F), 2′-O-methyl (2′-O-Me), 2′-O-methoxyethyl (2′-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2′-O—N-methylacetamido (2′-O-NMA)).

In some embodiments, an oligonucleotide described herein (e.g., a DMPK targeting oligonucleotide described herein) comprises a 5′-X—Y—Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, an oligonucleotide described herein (e.g., a DMPK targeting oligonucleotide described herein) comprises a 5′-X—Y—Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in Z (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside. In some embodiments, an oligonucleotide described herein (e.g., a DMPK targeting oligonucleotide described herein) comprises a 5′-X—Y—Z-3′ configuration, wherein X and Z are independently 2-7 (e.g., 2, 3, 4, 5, 6, or 7) nucleosides in length and Y is 6-10 (e.g., 6, 7, 8, 9, or 10) nucleosides in length, wherein at least one but not all (e.g., 1, 2, 3, 4, 5, or 6) of positions 1, 2, 3, 4, 5, 6, or 7 in X and at least one of positions but not all (e.g., 1, 2, 3, 4, 5, or 6) of positions 1, 2, 3, 4, 5, 6, or 7 in Z (the 5′-most position is position 1) is a non-bicyclic 2′-modified nucleoside (e.g., 2′-MOE or 2′-O-Me), wherein the rest of the nucleosides in both X and Z are 2′-4′ bicyclic nucleosides (e.g., LNA or cEt), and wherein each nucleoside in Y is a 2′deoxyribonucleoside.

In some embodiments, an oligonucleotide described herein (e.g., a DMPK targeting oligonucleotide) is 10-20 nucleosides (e.g., 10, 11, 12, 13, 14, 15, 16, 17, 18, 19, or 20 nucleosides) in length, comprises a region of complementarity to at least 8 consecutive nucleosides (e.g., at least 8, at least 9, at least 10, at least 11, at least 12, at least 13, at least 14, at least 15, or 16 consecutive nucleosides) of SEQ ID NO: 22 (TGACTGGTGGGCGCTG), and comprises a 5′-X—Y—Z-3′ configuration, wherein X comprises 3-5 (e.g., 3, 4, or 5) linked nucleosides, wherein at least one of the nucleosides in X is a 2′-modified nucleoside (e.g., 2′-MOE modified nucleoside, 2′-O-Me modified nucleoside, LNA, cEt, or ENA); Y comprises 6-10 (e.g., 6, 7, 8, 9, or 10) linked 2′-deoxyribonucleosides, wherein each cytosine in Y is optionally and independently a 5-methyl-cytosine; and Z comprises 3-5 (e.g., 3, 4, or 5) linked nucleosides, wherein at least one of the nucleosides in Z is a 2′-modified nucleoside (e.g., 2′-MOE modified nucleoside, 2′-O-Me modified nucleoside, LNA, cEt, or ENA).

In some embodiments, an oligonucleotide described herein (e.g., a DMPK targeting oligonucleotide described herein) comprises at least 8 consecutive nucleosides (e.g., at least 8, at least 9, at least 10, at least 11, at least 12, at least 13, at least 14, at least 15, or 16 consecutive nucleosides consecutive nucleosides) of the nucleotide sequence of SEQ ID NO: 21 (CAGCGCCCACCAGUCA), and comprises a 5′-X—Y—Z-3′ configuration, wherein X comprises 3-5 (e.g., 3, 4, or 5) linked nucleosides, wherein at least one of the nucleosides in X is a 2′-modified nucleoside (e.g., 2′-MOE modified nucleoside, 2′-O-Me modified nucleoside, LNA, cEt, or ENA); Y comprises 6-10 (e.g., 6, 7, 8, 9, or 10) linked 2′-deoxyribonucleosides, wherein each cytosine in Y is optionally and independently a 5-methyl-cytosine; and Z comprises 3-5 (e.g., 3, 4, or 5) linked nucleosides, wherein at least one of the nucleosides in Z is a 2′-modified nucleoside (e.g., 2′-MOE modified nucleoside, 2′-O-Me modified nucleoside, LNA, cEt, or ENA). In some embodiments, each thymine base (T) of the nucleotide sequence of the antisense oligonucleotide may independently and optionally be replaced with a uracil base (U), and each U may independently and optionally be replaced with a T.

In some embodiments, an oligonucleotide described herein (e.g., a DMPK targeting oligonucleotide described herein) comprises the nucleotide sequence of SEQ ID NO: 21 and comprises a 5′-X—Y—Z-3′ configuration, wherein X comprises 3-5 (e.g., 3, 4, or 5) linked nucleosides, wherein at least one of the nucleosides in X is a 2′-modified nucleoside (e.g., 2′-MOE modified nucleoside, 2′-O-Me modified nucleoside, LNA, cEt, or ENA); Y comprises 6-10 (e.g., 6, 7, 8, 9, or 10) linked 2′-deoxyribonucleosides, wherein each cytosine in Y is optionally and independently a 5-methyl-cytosine; and Z comprises 3-5 (e.g., 3, 4, or 5) linked nucleosides, wherein at least one of the nucleosides in Z is a 2′-modified nucleoside (e.g., 2′-MOE modified nucleoside, 2′-O-Me modified nucleoside, LNA, cEt, or ENA). In some embodiments, each thymine base (T) of the nucleotide sequence of the antisense oligonucleotide may independently and optionally be replaced with a uracil base (U), and each U may independently and optionally be replaced with a T.

In some embodiments, X comprises at least one 2′-4′ bicyclic nucleoside (e.g., LNA, cEt, or ENA) and at least one non-bicyclic 2′-modified nucleoside e.g., 2′-MOE modified nucleoside or 2′-O-Me modified nucleoside, and/or (e.g., and) Z comprises at least one 2′-4′ bicyclic nucleoside (e.g., LNA, cEt, or ENA) and at least one non-bicyclic 2′-modified nucleoside (e.g., 2′-MOE modified nucleoside or 2′-O-Me modified nucleoside).

In some embodiments, the 2′-4′ bicyclic nucleoside is selected from LNA, cEt, and ENA nucleosides. In some embodiments, the non-bicyclic 2′-modified nucleoside is a 2′-MOE modified nucleoside or a 2′-OMe modified nucleoside.

In some embodiments, the nucleosides of the oligonucleotides are joined together by phosphorothioate internucleoside linkages, phosphodiester internucleoside linkages or a combination thereof. In some embodiments, the oligonucleotide comprises only phosphorothioate internucleoside linkages joining each nucleoside (i.e., the oligonucleotide comprises a fully phosphorothioate backbone). In some embodiments, the oligonucleotide comprises at least one phosphorothioate internucleoside linkage. In some embodiments, the oligonucleotide comprises a mix of phosphorothioate and phosphodiester internucleoside linkages. In some embodiments, the oligonucleotide comprises only phosphorothioate internucleoside linkages joining each pair of 2′-deoxyribonucleosides and a mix of phosphorothioate and phosphodiester internucleoside linkages joining the remaining nucleosides.

In some embodiments, the oligonucleotide comprises a 5′-X—Y—Z-3′ configuration of LLEE-(D)₈-EELL, wherein “E” is a 2′-MOE modified ribonucleoside; “L” is LNA; “D” is a 2′-deoxyribonucleoside; and “10” or “8” is the number of 2′-deoxyribonucleosides in Y, and wherein the oligonucleotide comprises phosphorothioate internucleoside linkages, phosphodiester internucleoside linkages or a combination thereof.

In some embodiments, each cytidine (e.g., a 2′-modified cytidine) in X and/or Z of the oligonucleotide is optionally and independently a 5-methyl-cytidine, and/or each uridine (e.g., a 2′-modified uridine) in X and/or Z is optionally and independently a 5-methyl-uridine.

In some embodiments, an oligonucleotide described herein (e.g., a DMPK targeting oligonucleotide described herein) comprises a 5′-X—Y—Z-3′ configuration and comprises a nucleobase sequence of CAGCGCCCACCAGUCA (SEQ ID NO: 21). In some embodiments, an oligonucleotide described herein (e.g., a DMPK targeting oligonucleotide described herein) comprises a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage.

In some embodiments, an oligonucleotide described herein (e.g., a DMPK targeting oligonucleotide described herein) comprises a structure of the formula (Ie):

In some embodiments, an oligonucleotide described herein (e.g., a DMPK targeting oligonucleotide described herein) can be in salt form, e.g., as sodium, potassium, or magnesium salts.

In some embodiments, the 5′ or 3′ nucleoside (e.g., terminal nucleoside) of the oligonucleotide is conjugated to an amine group, optionally via a spacer. In some embodiments, the spacer comprises an aliphatic moiety. In some embodiments, the spacer comprises a polyethylene glycol moiety. In some embodiments, a phosphodiester linkage is present between the spacer and the 5′ or 3′ nucleoside of the oligonucleotide. In some embodiments, the 5′ or 3′ nucleoside (e.g., terminal nucleoside) of an oligonucleotide described herein is covalently linked to a spacer that is a substituted or unsubstituted aliphatic, substituted or unsubstituted heteroaliphatic, substituted or unsubstituted carbocyclylene, substituted or unsubstituted heterocyclylene, substituted or unsubstituted arylene, substituted or unsubstituted heteroarylene, —O—, —N(R^{A})—, —S—, —C(═O)—, —C(═O)O—, —C(═O)NR^{A}—, —NR^{A}C(═O)—, —NR^{A}C(═O)R^{A}—, —C(═O)R^{A}—, —NR^{A}C(═O)O—, —NR^{A}C(═O)N(R^{A})—, —OC(O)—, —OC(═O)O—, —OC(═O)N(R^{A})—, —S(O)₂NR^{A}—, —NR^{A}S(O)₂—, or a combination thereof; each R^{A} is independently hydrogen or substituted or unsubstituted alkyl. In certain embodiments, the spacer is a substituted or unsubstituted alkylene, substituted or unsubstituted heterocyclylene, substituted or unsubstituted heteroarylene, —O—, —N(R^{A})—, or —C(═O)N(R^{A})₂, or a combination thereof.

In some embodiments, the 5′ or 3′ nucleoside of the oligonucleotide is conjugated to a compound of the formula —NH₂—(CH₂)ₙ—, wherein n is an integer from 1 to 12. In some embodiments, n is 6, 7, 8, 9, 10, 11, or 12. In some embodiments, a phosphodiester linkage is present between the compound of the formula NH₂—(CH₂)ₙ- and the 5′ or 3′ nucleoside of the oligonucleotide. In some embodiments, a compound of the formula NH₂—(CH₂)₆— is conjugated to the oligonucleotide via a reaction between 6-amino-1-hexanol (NH₂—(CH₂)₆—OH) and the 5′ phosphate of the oligonucleotide.

In some embodiments, the oligonucleotide is conjugated to a targeting agent, e.g., a muscle targeting agent such as an anti-TfR1 antibody, e.g., via an amine group of a lysine of the targeting agent.

In some embodiments, it should be appreciated that methylation of the nucleobase uracil at the C5 position forms thymine. Thus, in some embodiments, a nucleotide or nucleoside having a C5 methylated uracil (or 5-methyl-uracil) may be equivalently identified as a thymine nucleotide or nucleoside.

In some embodiments, any one or more of the thymine bases (T's) in any one of the oligonucleotides provided herein may independently and optionally be uracil bases (U's), and/or any one or more of the U's in the oligonucleotides provided herein (e.g., the oligonucleotide as set forth in SEQ ID NO: 21) may independently and optionally be T's.

### Compositions

In some embodiments, compositions described herein comprise complexes (i.e., a plurality of complexes), each of which complex comprises an antibody (e.g., anti-TFR1 antibody) covalently linked to one or more oligonucleotides (e.g., a DMPK-targeting oligonucleotide described herein), wherein the antibody comprises a heavy chain comprising a heavy chain variable region (VH) and a heavy chain constant region, and a light chain comprising a light chain variable region (VL) and a light chain constant region. In some embodiments, the antibody of such complexes comprises a CDR-H1, CDR-H2, CDR-H3, CDR-L1, CDR-L2, and CDR-L3 as set forth in Table 2. Complexes of a composition described herein can comprise any structure provided herein, e.g., a structure of formula (I) (e.g., comprising a group of the formula (Ia), formula (Ib), formula (Ic), or formula (Id)) or formula (A).

In some embodiments, compositions described herein comprise complexes (i.e., a plurality of complexes) wherein each complex comprises a structure of formula (I): [R¹]ₙ₁—R², in which each R¹ independently comprises a compound comprising an oligonucleotide (e.g., a DMPK-targeting oligonucleotide described herein) and is covalently linked to R², wherein R² comprises an antibody (e.g., anti-TfR1 antibody) comprising a heavy chain comprising a heavy chain variable region (VH) and a heavy chain constant region, and a light chain comprising a light chain variable region (VL) and a light chain constant region. In some embodiments, each R¹ of a complex is independently covalently linked to a different amino acid residue (e.g., lysine or cysteine) of R².

In some embodiments, the value of n1 of complexes in the composition is independently and optionally an integer from one up to the number of amino acid residues to which conjugation is desired or targeted (e.g., the number of lysine residues) in the antibody (e.g., an antibody comprised within R²). In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the composition is independently and optionally selected from 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the composition is independently selected and optionally from an integer in the range of 1 to 27, 1 to 26, 1 to 10, 1 to 5, or 1 to 3. In some embodiments, the average value of n1 of complexes of the composition is in the range of 1 to 2, 1 to 3, 1 to 5, 1 to 10, 1 to 26, or 1 to 27. In some embodiments, compositions described herein comprise complexes in which the value of n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, a composition described herein comprises antibody that is not conjugated to an oligonucleotide (e.g., in trace amounts) and antibody conjugated to one or more oligonucleotides. In some embodiments, antibody that is not conjugated to an oligonucleotide may be referred to as a compound comprising a structure of formula (I): [R¹]ₙ₁—R², for which n1 is zero. Accordingly, in some embodiments, a composition for administration to a subject in the methods described herein comprises compounds (e.g., complexes) comprising a structure of formula (I): [R¹]ₙ₁—R², for which each R¹ independently comprises a group comprising an oligonucleotide, R² comprises an antibody and n1 is independently an integer of zero or greater that reflects the number of instances of R¹ in each compound (e.g., complex). In some embodiments, the fraction of compounds comprising a structure of formula (I): [R¹]ₙ₁—R², in a composition, for which n1 is zero, compared with all compounds of that structure in the composition for which n1 is one or greater, is less than 10%, less than 5%, less than 1% less than 0.5%, less than 0.1%, less than 0.05%, or less than 0.01%. As such, in some embodiments, the average value of n1 of complexes in a composition disclosed herein is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

### Formulations

Complexes provided herein are formulated in a manner suitable for pharmaceutical use. In some embodiments, complexes can be delivered to a subject using a formulation that minimizes degradation, facilitates delivery and/or (e.g., and) uptake, or provides another beneficial property to complexes in the formulation. Accordingly, in some embodiments, it has been found that formulating complexes (e.g., complexes comprising an oligonucleotide covalently linked with a Fab) with tris(hydroxymethyl)aminomethane (also known as tromethamine or THAM) and/or sucrose is particularly advantageous for pharmaceutical use, e.g., as described herein. Thus, in some embodiments, provided herein are formulations (e.g., aqueous solutions, lyophilized forms, or frozen forms) comprising complexes together with tris(hydroxymethyl)aminomethane and/or sucrose. Such formulations can be suitably prepared such that when administered to a subject, either into the immediate environment of a target cell or systemically, a sufficient amount of the complexes enter target muscle cells.

In some embodiments, formulations are provided herein that comprise complexes (i.e., a plurality of complexes) that comprise an oligonucleotide (e.g., an oligonucleotide comprising a 5′-X—Y—Z-3′ configuration) covalently linked to an antibody. In some embodiments, provided herein is a formulation comprising complexes, in which each complex comprises an oligonucleotide (e.g., an oligonucleotide comprising a 5′-X—Y—Z-3′ configuration) covalently linked to an anti-TfR1 antibody, optionally wherein the antibody of such complexes comprises a CDR-H1, CDR-H2, CDR-H3, CDR-L1, CDR-L2, and CDR-L3 as set forth in Table 2, and further, in some embodiments, wherein the complexes are formulated with tris(hydroxymethyl)aminomethane and sucrose. In some embodiments, the antibody is an anti-TfR1 antibody.

In some embodiments, formulations are provided that comprise complexes comprising a structure of formula (I): [R¹]ₙ₁—R², in which each R¹ independently comprises a compound comprising an oligonucleotide (e.g., e.g., an oligonucleotide comprising a 5′-X—Y—Z-3′ configuration) and R² comprises an antibody (e.g., anti-TfR1 antibody), and in which n1 is an integer representing the number of instances of R¹ in the complex.

In some embodiments, formulations described herein comprise complexes comprising an antibody that comprises a CDR-H1, CDR-H2, CDR-H3, CDR-L1, CDR-L2, and CDR-L3 as set forth in Table 2. In some embodiments, the antibody comprises a VH comprising the amino acid sequence of SEQ ID NO: 17 and a VL comprising the amino acid sequence of SEQ ID NO: 18. In some embodiments, the antibody is a Fab and comprises a heavy chain comprising the amino acid sequence of SEQ ID NO: 19 and a light chain comprising the amino acid sequence of SEQ ID NO: 20.

In some embodiments, a formulation described herein comprises complexes described herein at a concentration of between 1-50 mg of the complex per mL of the formulation. In some embodiments, a formulation described herein comprises complexes at a concentration of 10-50 mg/ml or 20-35 mg/mL (e.g., 15-45 mg/mL, 20-40 mg/mL, 25-35 mg/mL, 25.5-34.5 mg/mL, 26-34 mg/mL, 27-33 mg/mL, 28-32 mg/mL, 29-31 mg/mL, 29.5-30.5 mg/mL, 10-40 mg/mL, 15-35 mg/mL, 20-30 mg/mL, 21-29 mg/mL, 21.2-28.8 mg/mL, 22-28 mg/mL, 23-27 mg/mL, 24-26 mg/mL, or 24.5-25.5 mg/mL). In some embodiments, a formulation described herein comprises complexes at a concentration of approximately 25 mg/mL (e.g., 25 mg/mL). In some embodiments, a formulation described herein comprises complexes at a concentration of approximately 30 mg/mL (e.g., 30 mg/mL). In some embodiments, the concentration of complexes in a formulation may vary by up to 20% (e.g., +/−up to 20%, +/−up to 15%, +/−up to 10%, or +/−up to 5%) of a set value. For example, in some embodiments, the concentration of complexes in the formulation is 30 mg/mL+/−up to 15% (e.g., 30+/−4.5 mg/mL). In some embodiments, the concentration of complexes in the formula is 25 mg/mL+/−up to 15% (e.g., 25+/−3.8 mg/mL).

In some embodiments, any one or a plurality of the complexes described herein is formulated with tris(hydroxymethyl)aminomethane and sucrose in an aqueous solution. In some embodiments, the tris(hydroxymethyl)aminomethane is present in the aqueous solution at a concentration in the range of 5-50 mM (e.g., 5-40 mM, 5-35 mM, 5-30 mM, 10-50 mM, 15-45 mM, 10-40 mM, 20-40 mM, 20-35 mM, 20-30 mM, 21-29 mM, 22-28 mM, 23-27 mM, 24-26 mM). In some embodiments, the tris(hydroxymethyl)aminomethane is present in the aqueous solution at a concentration of approximately 25 mM (e.g., 25 mM). In some embodiments, the sucrose is present in the aqueous solution at a concentration in the range of 5% to 15% weight per volume (w/v %), for example, 7-13 w/v %, 8-15% w/v %, 9-15% w/v %, 9-11% w/v %, 9.5-11% w/v %, or for example, in the range of 9-10 w/v %, 10-11 w/v %, 10-12 w/v %, or 8-12 w/v %. In some embodiments, the sucrose is present in the aqueous solution at a concentration in the range of 7-13 w/v %, 8-12 w/v %, or 9-11 w/v %. In some embodiments, the sucrose is present in the aqueous solution at a concentration of approximately 10 w/v % (e.g., 10 w/v %). In some embodiments, the aqueous solution has a pH in the range of 6.5 to 8.5, for example, 6.5-6.5, 6.7-6.9, 6.9-7.1, 7.1-7.3, 7.2-7.8, 7.3-7.5, 7.4-7.5, 7.4-7.6, 7.5-7.6; for example, 7.0-8.0, or for example, in the pH range of 7.0-7.3, 7.2-7.8, 7.3-7.5, 7.4-7.6, 7.5-7.6, 7.5-7.7, 7.7-7.9, 7.9-8.0, 8.0-8.2, 8.2-8.4, 8.3-8.4, 8.4-8.5, 8.5-8.6, or 7.3-7.7. In some embodiments, the aqueous solution has a pH in the range of 7.0-8.0 (e.g., 7.0-7.8, 7.1-7.8, 7.2-7.8, 7.3-7.7, 7.3-7.5, 7.3-7.6, 7.4-7.6, or 7.4-7.8). In some embodiments, the aqueous solution has a pH of approximately 7.5 (e.g., 7.5). In some embodiments, the aqueous solution has a pH in the range of 7.4-7.7. In some embodiments, the aqueous solution has a pH in the range of 7.4-7.6 (e.g., 7.5, or about 7.5).

In some embodiments, any one of the formulations described herein is an aqueous solution, wherein tris(hydroxymethyl)aminomethane is present in the aqueous solution at a concentration of approximately 25 mM (e.g., 25 mM), wherein sucrose is present in the aqueous solution at a concentration of approximately 10 w/v % (e.g., 10 w/v %), and wherein the aqueous solution is at a pH of approximately 7.5 (e.g., 7.5).

In some embodiments, any one of the formulations described herein is an aqueous solution, wherein tris(hydroxymethyl)aminomethane is present in the aqueous solution at a concentration of approximately 25 mM (e.g., 25 mM), wherein sucrose is present in the aqueous solution at a concentration of approximately 10 w/v % (e.g., 10 w/v %), wherein complexes are present in the aqueous solution at a concentration of approximately 20-35 mg/ml (e.g., 25 mg/ml or 30 mg/ml) and wherein the aqueous solution is at a pH of approximately 7.5 (e.g., 7.5).

In some embodiments, any one or a plurality of the complexes described herein is formulated with tris(hydroxymethyl)aminomethane and sucrose in a lyophilized form (e.g., lyophilized powder). In some embodiments, the lyophilized form (e.g., lyophilized powder) is obtained by lyophilization of any one of the aqueous solutions described herein.

In some embodiments, a lyophilized form is a lyophilized cake. In some embodiments, a lyophilized cake comprises a plurality of complexes provided herein, tris(hydroxymethyl)aminomethane, and sucrose. In some embodiments, a lyophilized cake comprises 1×10⁻² mg-1.5 mg (e.g., 1×10⁻² mg-1.5 mg, 4.02×10⁻² mg-1.21 mg, 6×10⁻² mg-1 mg, 8×10⁻² mg-1 mg, 0.1 mg-1 mg, 0.05 mg-0.2 mg, 0.05 mg-0.3 mg, 0.05 mg-0.4 mg, 0.1 mg-0.2 mg, 0.1 mg-0.3 mg, 0.1 mg-0.4 mg, 0.1 mg-0.5 mg, 0.2 mg-0.3 mg, 0.2 mg-0.4 mg, 0.2 mg-0.4 mg, 0.3 mg-0.4 mg, or 0.3 mg-0.5 mg) tris(hydroxymethyl)aminomethane per g of cake. In some embodiments, a lyophilized cake comprises 900 mg-1100 mg (e.g., 900 mg-1100 mg, 989 mg-999 mg, 950 mg-1000 mg, 980 mg-1000 mg, 980 mg-990 mg, 990 mg-1000 mg, 995 mg-1000 mg, 992 mg-998 mg, 994 mg-999 mg) sucrose per g of cake. In some embodiments, a lyophilized cake comprises 0.5 mg-10 mg (e.g., 0.5 mg-10 mg, 0.5 mg-1.5 mg, 1 mg-2 mg, 1.5 mg-3 mg, 1 mg-8 mg, 0.5 mg-6 mg, 0.5 mg-5 mg, 1.5 mg-3.5 mg, 1.5 mg-4 mg, 1.5 mg-5 mg, 2 mg-5 mg, 2.5 mg-5 mg, 2 mg-3 mg, 3 mg-4 mg, 4 mg-5 mg) of complexes per g of cake.

In some embodiments, a lyophilized cake comprises 0.303 mg tris(hydroxymethyl)aminomethane per g of cake, 998.6 mg sucrose per g of cake, and/or (e.g., and) 0.999 mg complexes per g of cake. In some embodiments, a lyophilized cake comprises 0.302 mg tris(hydroxymethyl)aminomethane per g of cake, 997.7 mg sucrose per g of cake, and/or (e.g., and) 1.995 mg complexes per g of cake. In some embodiments, a lyophilized cake comprises 0.302 mg tris(hydroxymethyl)aminomethane per g of cake, 997.2 mg sucrose per g of cake, and/or (e.g., and) 2.49 mg complexes per g of cake. In some embodiments, a lyophilized cake comprises 0.302 mg tris(hydroxymethyl)aminomethane per g of cake, 996.7 mg sucrose per g of cake, and/or (e.g., and) 2.99 mg complexes per g of cake. In some embodiments, a lyophilized cake comprises 0.302 mg tris(hydroxymethyl)aminomethane per g of cake, 995.7 mg sucrose per g of cake, and/or (e.g., and) 3.98 mg complexes per g of cake. In some embodiments, a lyophilized cake comprises 0.301 mg tris(hydroxymethyl)aminomethane per g of cake, 994.7 mg sucrose per g of cake, and/or (e.g., and) 4.97 mg complexes per g of cake.

In some embodiments, reconstitution of a lyophilized cake (e.g., with water) results in a solution comprising tris(hydroxymethyl)aminomethane at a concentration of 5 to 50 mM, sucrose at a concentration of 5 w/v % to 15 w/v %, and/or (e.g., and) complexes at a concentration of 10 mg/mL to 50 mg/mL. In some embodiments, reconstitution of 10.053 g of a lyophilized cake with a solvent (e.g., water) to form 1 mL of reconstituted solution results in a solution comprising tris(hydroxymethyl)aminomethane at a concentration of 5 to 50 mM (optionally 25 mM), sucrose at a concentration of 5 w/v % to 15 w/v % (optionally 10 w/v %), and/or (e.g., and) complexes at a concentration of 10 mg/mL to 50 mg/mL. In some embodiments, reconstitution of about 10 g of a lyophilized cake with a solvent (e.g., water) to form 1 mL of reconstituted solution results in a solution comprising tris(hydroxymethyl)aminomethane at a concentration of 5 to 50 mM (optionally 25 mM), sucrose at a concentration of 5 w/v % to 15 w/v % (optionally 10 w/v %), and/or (e.g., and) complexes at a concentration of 10 mg/mL to 50 mg/mL.

In some embodiments, any one or a plurality of the complexes described herein is formulated with tris(hydroxymethyl)aminomethane and sucrose in a frozen form (e.g., a frozen aqueous solid). In some embodiments, the frozen form (e.g., frozen aqueous solid) is obtained by freezing of any one of the aqueous solutions described herein. A frozen form may be frozen to a temperature of less than −20° C. (e.g., less than −20° C., less than −30° C., less than −40° C., less than −50° C., less than −60° C., less than −70° C., less than −80° C., or lower).

In some embodiments, the value of n1 of each complex in the formulation is independently and optionally an integer from zero up to the number of amino acid residues to which conjugation is desired or targeted (e.g., the number of lysine residues) in the antibody (R²). In some embodiments, the value of n1 of each complex in the formulation is independently and optionally selected from 0, 1, 2, 3, 4, 5, 6, 7, 8, 9, 10, 11, 12, 13, 14, 15, 16, 17, 18, 19, 20, 21, 22, 23, 24, 25, 26, and 27. In some embodiments, the value of n1 of each complex in the formulation is independently and optionally selected from 0, 1, 2, 3, 4, 5, 6, 7, 8, 9, 10, 11, 12, 13, 14, 15, 16, 17, 18, 19, 20, 21, 22, 23, 24, 25 and 26. In some embodiments, the value of n1 of each complex in the formulation is independently selected and optionally from an integer in the range of 1 to 27, 1 to 26, 1 to 10, 1 to 5, or 1 to 3. In some embodiments, the average value of n1 of complexes in the formulation is in the range of 1 to 2, 1 to 3, 1 to 5, 1 to 10, 1 to 26 or 1 to 27. In some embodiments, formulations described herein comprise complexes that comprise a structure of formula (I): [R¹]ₙ₁—R², wherein n1 is 0. In some embodiments, the average value of n1 of complexes of the formulation is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2).

In some embodiments, each instance of R¹ in a complex herein (e.g., a complex of a formulation provided herein) is conjugated to a different amino acid residue of the antibody. In some embodiments, each different amino acid comprises an F-amino group (e.g., lysine, arginine). However, in some embodiments, each different amino acid to which R¹ is covalently linked is a cysteine. In some embodiments, R¹ is directly covalently linked to an amino acid residue of the antibody. However, in some embodiments, R¹ is indirectly covalently linked to an amino acid of the antibody, e.g., covalently linked to a glycosylation site on the amino acid. In some embodiments, formulations are provided in which complexes for which R¹ is covalently linked to an amino acid residue residing in a CDR region of the antibody are present in only trace amounts, or in undetectable amount, or not at all. In some embodiments, formulations are provided in which complexes for which R¹ is covalently linked to an amino acid residue residing in a CDR region of the antibody are not detectable in the formulation using standard detection techniques.

In some embodiments, formulations provided herein comprise complexes that comprise a structure of formula (I): [R¹]ₙ₁—R², in which each instance of R¹ independently comprises a group of the formula (Ia):

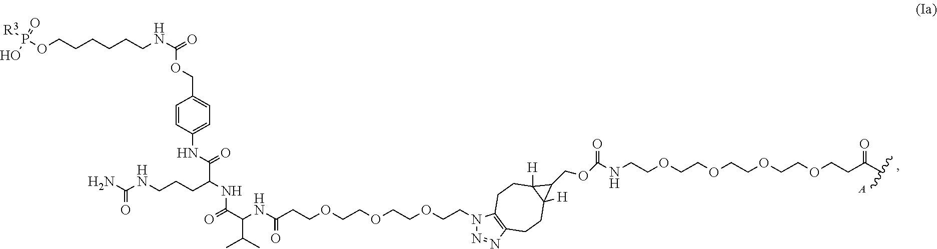

in which R³ comprises an oligonucleotide, e.g., an oligonucleotide comprising a 5′-X—Y—Z-3′ configuration; and R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, R³ comprises an oligonucleotide comprising a nucleobase sequence of CAGCGCCCACCAGUCA (SEQ ID NO: 21). In some embodiments, R³ comprises an oligonucleotide comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage. In some embodiments, in each complex n1 is independently an integer (e.g., an integer in the range of 1-27, 1-26, 1-10, 1-5, or 1-3). In some embodiments, formulations described herein further comprise complexes that comprise a structure of formula (I): [R¹]ₙ₁—R², wherein n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

In some embodiments, formulations provided herein comprise complexes that comprise a structure of formula (I): [R¹]ₙ₁—R², in which each instance of R¹ in a complex of a formulation provided herein comprises a group of the formula (Ib):

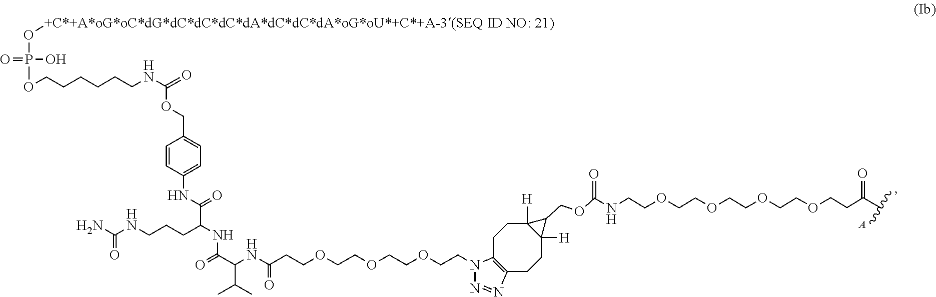

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21), wherein n1 is an integer (e.g., one or greater) representing the number of instances of R¹ in each complex, and each R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, formulations described herein further comprise complexes that comprise a structure of formula (I): [R¹]ₙ₁—R², wherein n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

In some embodiments, formulations provided herein comprise complexes that comprise a structure of formula (I): [R¹]ₙ₁—R², in which each R¹ comprises a group of the formula (Ic):

wherein R¹ is covalently linked to R² at attachment point A. In some embodiments, R² comprises an antibody comprising a sequence as set forth in Table 2. For example, in some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, formulations described herein further comprise complexes that comprise a structure of formula (I): [R¹]ₙ₁—R², wherein n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

In some embodiments, formulations provided herein comprise complexes that comprise a structure of the formula (Id):

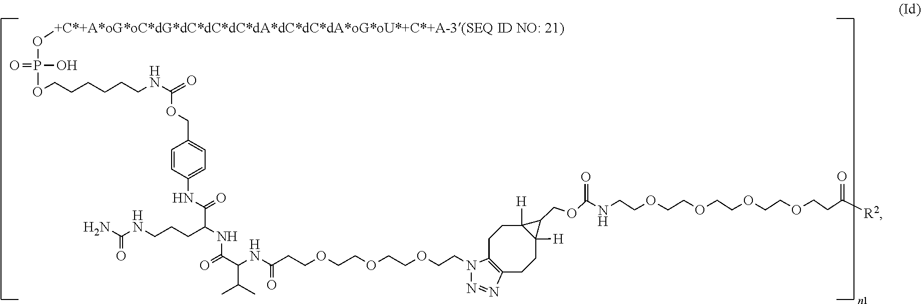

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21); wherein R² comprises an antibody comprising a sequence as set forth in Table 2; wherein n1 is an integer (e.g., one or greater) representing the number of instances of the group enclosed by square brackets, wherein each instance of the group enclosed by square brackets is covalently linked to a different amino acid residue of the antibody, optionally wherein each different amino acid residue is a lysine. In some embodiments, R² comprises an antibody comprising a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprising a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, R² comprises an antibody comprising a heavy chain variable region (VH) comprising an amino acid sequence at least 85% (e.g., at least 95%) identical to SEQ ID NO: 17 and/or comprising a light chain variable region (VL) comprising an amino acid sequence at least 85% (e.g., at least 95%) identical to SEQ ID NO: 18. In some embodiments, R² comprises an antibody comprising a VH comprising the amino acid sequence of SEQ ID NO: 17 and/or comprising a VL comprising the amino acid sequence of SEQ ID NO: 18. In some embodiments, R² comprises an antibody comprising a heavy chain comprising an amino acid sequence at least 85% (e.g., at least 95%) identical to SEQ ID NO: 19 and/or comprising a light chain comprising an amino acid sequence at least 85% (e.g., at least 95%) identical to SEQ ID NO: 20. In some embodiments, R² comprises an antibody comprising a heavy chain comprising the amino acid sequence of SEQ ID NO: 19 and/or comprising a light chain comprising the amino acid sequence of SEQ ID NO: 20. In some embodiments, R² comprises an antibody that is a Fab fragment, a full-length IgG, a Fab′ fragment, a F(ab′)₂ fragment, an scFv, or an Fv. In some embodiments, formulations described herein further comprise complexes in which n1 is 0. In some embodiments, the average value of n1 of complexes of the composition is in the range of 0.5 to 5 (e.g., 0.5-5, 1-5, 1-4, 1-3, 3-5, 0.5-4, 0.5-3, 0.5-2, 0.5-1.5, 0.5-1, 0.7-1.5, 1-1.6, 1-1.5, 1-1.4, 1-1.3, 1-1.2, 1.1-1.5, 0.8-2, 0.8-1.5, 0.8-1.3, 0.8-1.2, 0.8-1.1, 0.9-3, 0.9-2, 0.9-1.8, 0.9-1.6, 0.9-1.5, 0.9-1.4, 0.9-1.3, or 0.9-1.2). In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

In some embodiments, complexes provided in the formulations described herein comprise a structure of formula (A):

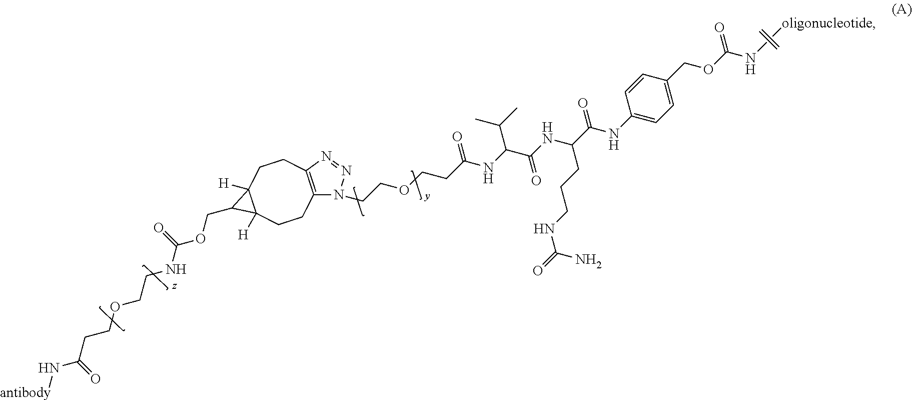

(A), wherein y is 0-15 (e.g., 3) and z is 0-15 (e.g., 4). In some embodiments, the amide shown adjacent the antibody (e.g., anti-TfR1 antibody) in the structure (A) results from a reaction with an amine of the antibody, such as a lysine epsilon amine. In some embodiments, a complex described herein comprises an anti-TfR1 antibody (e.g., an anti-TfR1 Fab) covalently linked via a lysine of the antibody to the 5′ end of an oligonucleotide (e.g., an oligonucleotide comprising a 5′-X—Y—Z-3′ configuration). In some embodiments, the antibody comprises a sequence as set forth in Table 2. For example, in some embodiments, the antibody comprises a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14; and/or comprises a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5, or 11, and a light chain complementarity determining region 3 (CDR-L3) comprising a sequence as set forth in SEQ ID NO: 6 or 16. In some embodiments, the antibody comprises a heavy chain variable region (VH) comprising an amino acid sequence at least 85% (e.g., at least 95%) identical to SEQ ID NO: 17 and/or comprises a light chain variable region (VL) comprising an amino acid sequence at least 85% (e.g., at least 95%) identical to SEQ ID NO: 18. In some embodiments, the antibody comprises a VH comprising the amino acid sequence of SEQ ID NO: 17 and/or comprises a VL comprising the amino acid sequence of SEQ ID NO: 18. In some embodiments, the antibody comprises a heavy chain comprising an amino acid sequence at least 85% (e.g., at least 95%) identical to SEQ ID NO: 19 and/or comprises a light chain comprising an amino acid sequence at least 85% (e.g., at least 95%) identical to SEQ ID NO: 20. In some embodiments, the antibody comprises a heavy chain comprising the amino acid sequence of SEQ ID NO: 19 and/or comprises a light chain comprising the amino acid sequence of SEQ ID NO: 20. In some embodiments, the antibody is a Fab fragment, a full-length IgG, a Fab′ fragment, a F(ab′)₂ fragment, an scFv, or an Fv. In some embodiments, the antibody is a Fab fragment. In some embodiments, formulations further comprise tris(hydroxymethyl)aminomethane and sucrose. In some embodiments, formulations comprise tris(hydroxymethyl)aminomethane at a concentration of 25 mM and/or (e.g., and) sucrose at a concentration of 10 w/v %, optionally wherein the formulation has a pH of 7.5. In some embodiments, formulations comprise complexes at a concentration of 30 mg/mL.

As described herein, in some embodiments, formulations provided herein comprise sucrose. In some embodiments, sucrose serves at least in part as a lyoprotectant. In some embodiments, the sucrose is from a plant, e.g., grass, fruit, or vegetable (e.g., root vegetable) source (e.g., beet (e.g., sugar beet, for example, Saccharum spp.)), sugarcane (e.g., Beta vulgaris), dates, sugar maple, sweet sorghum, apples, oranges, carrots, molasses, maple syrup, corn sweeteners) or animal product (e.g., honey). In some embodiments, the sucrose is from beet or sugarcane (e.g., beet sucrose, sugarcane sucrose). In some embodiments, a lyoprotectant other than sucrose may be used, e.g., trehalose, mannitol, lactose, polyethylene glycol, or polyvinyl pyrrolidone. However, in some embodiments, a collapse temperature modifier (e.g., dextran, ficoll, or gelatin) may be provided in a formulation.

In some embodiments, provided is a product (e.g., lyophilized formulation described herein), produced by a process comprising lyophilizing an aqueous solution of a formulation (e.g., aqueous form) described herein.

In some embodiments, a pharmaceutical composition is formulated to be compatible with its intended route of administration. Examples of routes of administration include parenteral, e.g., intravenous, intradermal, subcutaneous, administration. Typically, the route of administration is intravenous or subcutaneous.

### Methods of Use/Treatment

Complexes comprising an anti-TfR1 antibody (e.g., a Fab) covalently linked to a molecular payload (e.g., a DMPK targeting oligonucleotide) as described herein are effective in treating a subject having a myotonic dystrophy, e.g., DM1. In some embodiments, complexes comprise a molecular payload that is an oligonucleotide, e.g., an oligonucleotide that facilitates reduced expression or activity of DMPK (e.g., reduced level of a mutant or wild-type DMPK RNA).

In some embodiments, a subject may be a human subject, a non-human primate subject, a rodent subject, or any suitable mammalian subject. In some embodiments, a subject may have myotonic dystrophy. In some embodiments, a subject has a DMPK allele, which may optionally contain a disease-associated repeat, e.g., a CTG trinucleotide repeat expansion. In some embodiments, a subject may have a DMPK allele with an expanded disease-associated-repeat that comprises about 2-10 repeat units, about 2-50 repeat units, about 2-100 repeat units, about 50-1,000 repeat units, about 50-500 repeat units, about 50-250 repeat units, about 50-100 repeat units, about 500-10,000 repeat units, about 500-5,000 repeat units, about 500-2,500 repeat units, about 500-1,000 repeat units, or about 1,000-10,000 repeat units. In some embodiments, a subject may have myotonic dystrophy, such as DM1. In some embodiments, a subject is suffering from symptoms of DM1, e.g. muscle atrophy, muscle loss, excessive daytime sleepiness or cognitive delay. In some embodiments, a subject is not suffering from symptoms of DM1. In some embodiments, subjects have congenital myotonic dystrophy. In some embodiments, a subject is ambulant. In some embodiments, a subject is non-ambulant.

An aspect of the disclosure includes methods involving administering to a subject a formulation comprising an effective amount of complex(es) as described herein. In some embodiments, an effective amount of a pharmaceutical composition that comprises complex(es) comprising an antibody (e.g., Fab) described herein covalently linked to an oligonucleotide (e.g., a DMPK targeting oligonucleotide) described herein can be administered to a subject in need of treatment. In some embodiments, a pharmaceutical composition comprising complex(es) as described herein may be administered by a suitable route, which may include intravenous administration, e.g., as a bolus or by continuous infusion over a period of time. In some embodiments, administration may be performed by intravenous, intramuscular, intraperitoneal, intracerebrospinal, subcutaneous, intra-articular, intrasynovial, or intrathecal routes. In some embodiments, a pharmaceutical composition may be in solid form, aqueous form, or a liquid form. In some embodiments, an aqueous or liquid form may be nebulized or lyophilized. In some embodiments, a lyophilized form may be reconstituted with an aqueous or liquid solution.

In some embodiments, provided are methods of and/or uses for treating a subject having a DMPK allele, which may optionally contain a disease-associated repeat, comprising administering to the subject a formulation described herein that comprises an effective amount of complex(es) described herein. In some embodiments, provided are methods of and/or uses for reducing the expression or activity of DMPK (e.g., reducing the level of a mutant or wild-type DMPK RNA, or the activity of a DMPK gene product) in a cell (e.g., a muscle cell), the methods comprising contacting the cell with a formulation described herein comprising an effective amount of complex(es) described herein. In some embodiments, the method comprises administering a lyophilized form (e.g., lyophilized powder) of the formulation described herein, comprising reconstituting a lyophilized form of the formulation in an aqueous solution, and administering the aqueous solution of the formulation to a subject in need thereof. For example, in some embodiments, a lyophilized form of the formulation is shipped and/or stored in the lyophilized form, reconstituted at a location for administering the aqueous solution of the formulation (e.g., healthcare provider location), and administered in the reconstituted form (e.g., as an aqueous solution) by injection or intravenously, e.g., by infusion.

In some embodiments, a pharmaceutical composition is administered via site-specific or local delivery techniques. Examples of these techniques include implantable depot sources of the complex, local delivery catheters, site specific carriers, direct injection, or direct application.

In some embodiments, a pharmaceutical composition that comprises a complex comprising a an anti-TfR1 antibody (e.g., a fab) covalently linked to a molecular payload (e.g., a DMPK targeting oligonucleotide) is administered at an effective concentration that confers therapeutic effect on a subject. Effective amounts vary, as recognized by those skilled in the art, depending on the severity of the disease, unique characteristics of the subject being treated, e.g. age, physical conditions, health, or weight, the duration of the treatment, the nature of any concurrent therapies, the route of administration and related factors. These related factors are known to those in the art and may be addressed with no more than routine experimentation. In some embodiments, an effective concentration is the maximum dose that is considered to be safe for the patient. In some embodiments, an effective concentration will be the lowest possible concentration that provides maximum efficacy.

Empirical considerations, e.g. the half-life of the complex(es) in a subject, generally will contribute to determination of the concentration of pharmaceutical composition that is used for treatment. The frequency of administration may be empirically determined and adjusted to maximize the efficacy of the treatment. The efficacy of treatment may be assessed using any suitable methods. In some embodiments, the efficacy of treatment may be assessed by evaluation or observation of symptoms associated with a myotonic dystrophy, e.g. muscle atrophy or muscle weakness, through measures of a subject's self-reported outcomes, e.g. mobility, self-care, usual activities, pain/discomfort, and anxiety/depression, or by quality-of-life indicators, e.g. lifespan. In some embodiments, a pharmaceutical composition that comprises a complex comprising a muscle-targeting agent covalently linked to a molecular payload described herein is administered to a subject at an effective concentration sufficient to modulate activity or expression of a target gene by at least 10%, at least 20%, at least 30%, at least 40%, at least 50%, at least 60%, at least 70%, at least 80%, at least 90% or at least 95% relative to a control, e.g. baseline level of gene expression prior to treatment.

### EXAMPLES

### Example 1. In Vivo Tissue Distribution of Conjugates Containing Anti-TfR1 Fab Conjugated to a DMPK-Targeting Oligonucleotide in DM1 Mouse Model

Conjugates comprising an anti-TfR1 Fab conjugated to a DMPK-targeting oligonucleotide (ASO) were tested in a mouse model that expresses both human TfR1 and a human DMPK mutant that harbors expanded CUG repeats. The anti-TfR1 Fab used has the VH/VL sequences provided in Table 2. The Fab was covalently linked (through lysine conjugation) via a linker comprising a valine-citrulline sequence to a DMPK targeting oligonucleotide comprising a nucleobase sequence of SEQ ID NO: 21. The conjugate comprises a structure of formula (Id):

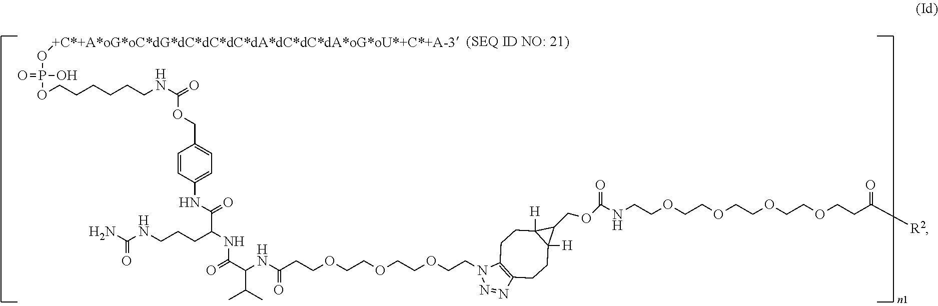

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, and wherein the oligonucleotide comprises a nucleobase sequence of CAGCGCCCACCAGUCA (SEQ ID NO: 21), and in which R² comprises the anti-TfR1 antibody provided in Table 2, and wherein in each conjugate n1 is independently an integer of 1-3.

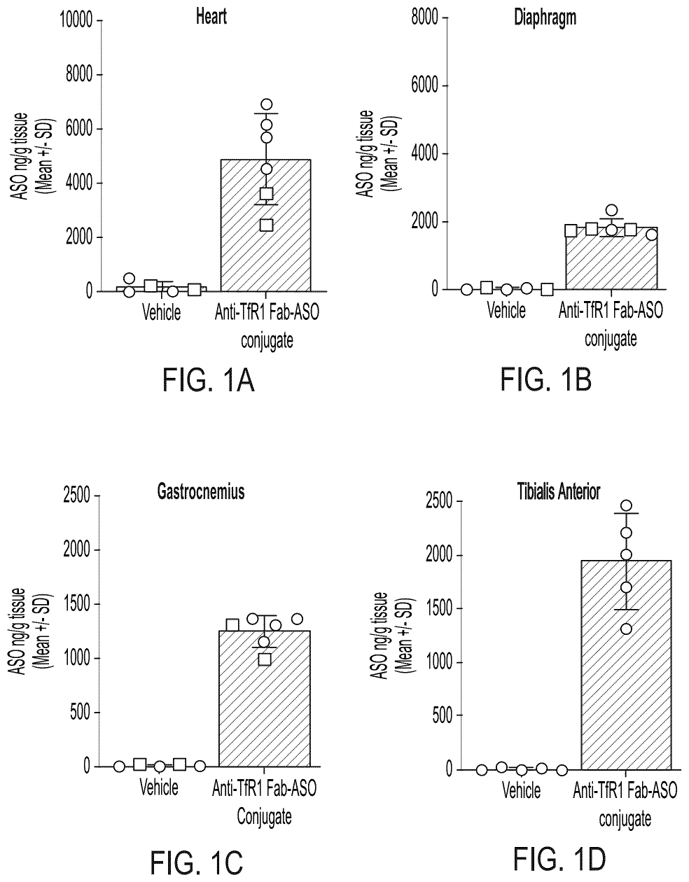

The conjugates were administered intravenously to the mice at day 0 and day 7, each time at a dose equivalent to 9.7 mg/kg of the ASO. The tissue exposure of the ASO was tested by hybridization ELISA (Burki et al., Nucleic Acid Ther. 2015 October; 25(5):275-84, incorporated herein by reference), and the levels of ASO in the tissue were graphed. FIGS. 1A, 1B, 1C, and 1D show the amount of ASO in the heart, diaphragm, gastrocnemius, or tibialis anterior, respectively, two weeks after the first injection. These results demonstrate that conjugates comprising an anti-TfR1 antibody (e.g., an anti-TfR1 Fab having the VH and VL sequences provided in Table 2) are capable of delivering an oligonucleotide (e.g., a DMPK-targeting ASO) to various muscle tissues following intravenous administration.

### Example 2. Sustained Knockdown of Toxic Human DMPK in hTfR1/DMSXL Homozygous Mice at 4 Weeks after Repeat Dosing of Anti-TfR1 Fab-ASO Conjugates

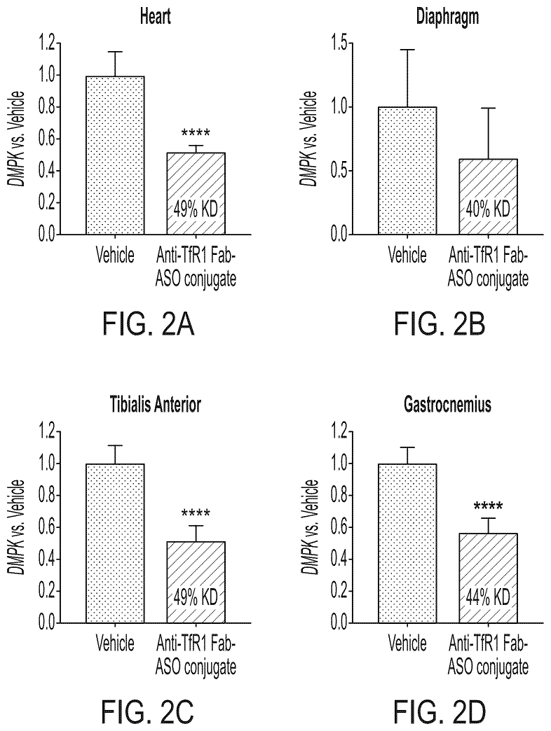

Conjugates (labeled in this Example as “Anti-TfR1 Fab-ASO conjugate”) as described in Example 1, containing an anti-TfR1 Fab covalently linked to a DMPK-targeting oligonucleotide (ASO) were tested in a mouse model that expresses both human TfR1 and two copies of a mutant human DMPK transgene that harbors expanded CUG repeats (hTfR1/DMSXL mice). Mice were administered either vehicle control (PBS) or 10 mg/kg ASO-equivalent dose of anti-TfR1 Fab-ASO conjugate at days 0 and 7. Mice were sacrificed at day 28 (four weeks following administration of the first dose of anti-TfR1 Fab-ASO conjugate), and tissues were collected. RNA was extracted and selected tissue samples were fixed, paraffin embedded and sectioned, then subjected to in situ hybridization. Reverse transcription-quantitative polymerase chain reaction (RT-qPCR) of the RNA samples was performed to measure human DMPK and mouse Ppib (peptidylprolyl isomerase) as an internal control. DMPK expression is shown in FIGS. 2A-2D as geometric means+/−standard deviation (n=6−9). Significance was assessed by Student's t-test (**** P<0.0001).

FIG. 2A shows that anti-TfR1 Fab-ASO conjugate knocked down DMPK expression in heart by 49% relative to PBS-treated mice. FIG. 2B shows that anti-TfR1 Fab-ASO conjugate knocked down DMPK expression in diaphragm by 40% relative to PBS-treated mice. FIG. 2C shows that anti-TfR1 Fab-ASO conjugate knocked down DMPK expression in tibialis anterior by 49% relative to PBS-treated mice. FIG. 2D shows that anti-TfR1 Fab-ASO conjugate knocked down DMPK expression in gastrocnemius by 44% relative to PBS-treated mice.

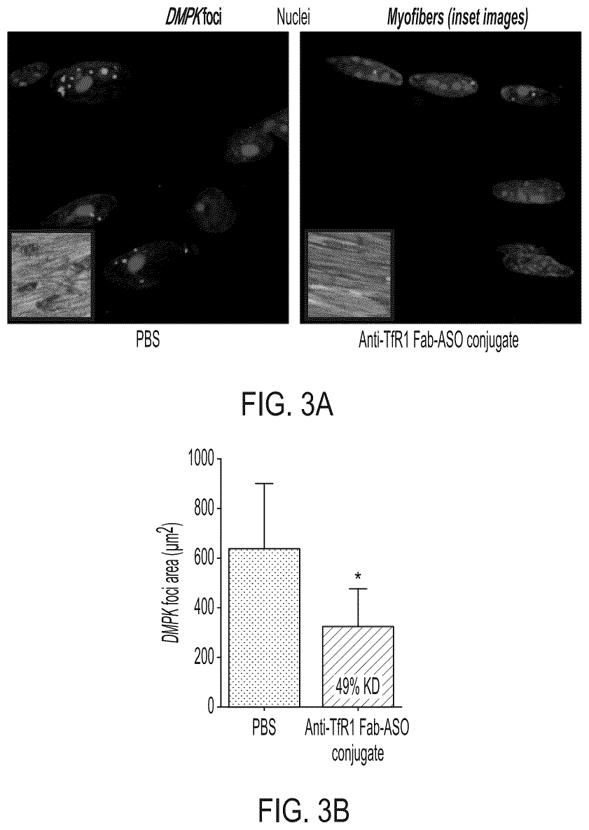

FIGS. 3A and 3B show that anti-TfR1 Fab-ASO conjugate reduced DMPK foci within nuclei of myofibers. FIG. 3A shows reduced DMPK foci by in situ hybridization, and FIG. 3B shows quantification of DMPK foci in fluorescent microscopy images, demonstrating the conjugate reduced foci area by 49%. Data are presented as mean+/−standard deviation (n=7). Significance was assessed by t-test (*P<0.05).

These results demonstrate that administration of anti-TfR1 Fab-ASO conjugate leads to robust, sustained knockdown of human toxic DMPK in cardiac and skeletal muscle.

### Example 3. Correction of Splicing Defects in hTfR1/DMSXL Homozygous Mice by Anti-TfR1 Fab-ASO Conjugates

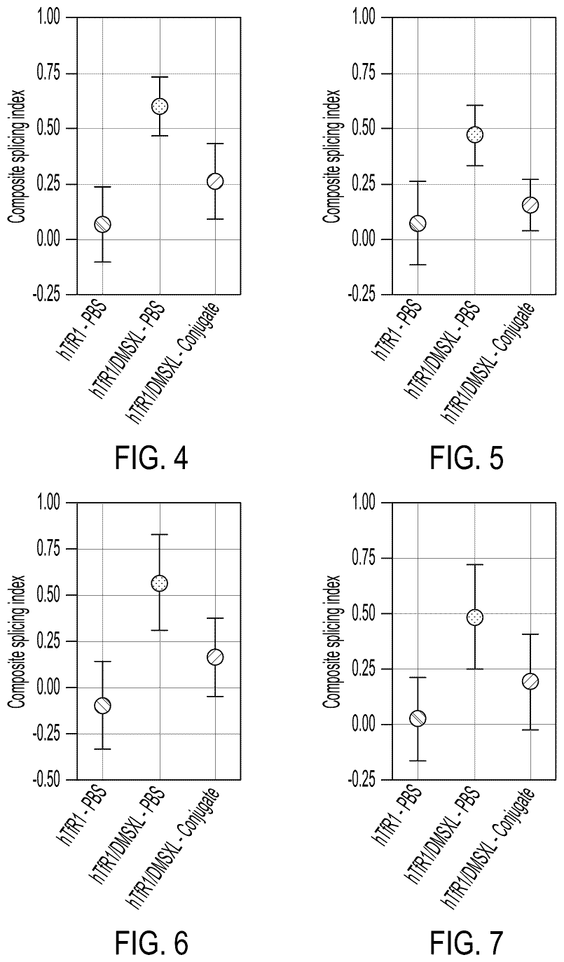

Conjugates (labeled in this Example as “Anti-TfR1 Fab-ASO conjugate”) as described in Example 1, containing an anti-TfR1 Fab covalently linked to a DMPK-targeting oligonucleotide (ASO) were tested in a mouse model (“hTfR1/DMSXL”) that expresses both human TfR1 and two copies of a mutant human DMPK transgene that harbors expanded CUG repeats. These mice are known to display splicing defects that are consistent with those observed in patients afflicted with DM1 (Huguet, et al. (2012) PLOS Genetics 8(11): e1003043). Mice were administered either vehicle control (“hTfR1/DMSXL—PBS”) or 10 mg/kg ASO-equivalent dose of anti-TfR1 Fab-ASO conjugate (“hTfR1/DMSXL—Conjugate”) on days 0 and 7. Mice expressing only the human TfR1 but not the mutant human DMPK transgene (hTfR1 mice) and treated with PBS (“hTfR1—PBS”) were used as another control to define the extent of the splicing phenotype in hTfR1/DMSXL mice and assess the magnitude of the effect of the conjugate on splicing. Mice were sacrificed on day 28 (four weeks following administration of the first dose of anti-TfR1 Fab-ASO conjugate), tissues were collected, and RNA was extracted. Reverse transcription-quantitative polymerase chain reaction (RT-qPCR) was performed to measure exon inclusion in a set of RNAs known to be mis-spliced during DM1 progression in humans and mice (Nakamori, et al. (2013) Ann. Neurol. 74(6): 862-872; Huguet, et al. (2012) PLOS Genetics 8(11): e1003043). Exon inclusion was calculated as normalized percent spliced in (PSI) for each splicing RNA marker, and composite splicing indices were calculated using the normalized PSI values from splicing markers in heart (FIG. 4), diaphragm (FIG. 5), tibialis anterior (FIG. 6), and gastrocnemius (FIG. 7). Composite splicing indices were calculated as previously described (Tanner M K, et al. (2021) Nucleic Acids Res. 49:2240-2254), and are shown as mean+/−standard deviation.

FIG. 4 shows that anti-TfR1 Fab-ASO conjugate corrected splicing in heart tissue of hTfR1/DMSXL mice, as demonstrated by composite splicing index data. The normalized PSI values used to generate the composite splicing index data showed correction of Mbnl2 exon 6 (E6) and Nfix E7 splicing in heart tissue of hTfR1/DMSXL mice by treatment with anti-TfR1 Fab-ASO conjugate, but did not show correction of Ldb3 E11 splicing. Composite splicing index data shown in FIG. 4 were based on Ldb3 E11, Mbnl2 E6, and Nfix E7 splicing data; Bin1 E11, Dtna E12, Insr E11, and Mbnl2 E5 were not included because their normalized PSI values in heart tissue were not changed in hTfR1/DMSXL mice relative to hTfR1 mice under the experimental conditions tested.

FIG. 5 shows that anti-TfR1 Fab-ASO conjugate corrected splicing in diaphragm tissue of hTfR1/DMSXL mice, as demonstrated by composite splicing index data. The normalized PSI values used to generate the composite splicing index data showed correction of Bin1 E11, Insr E11, Ldb3 E11 and Nfix E7 splicing in diaphragm tissue of hTfR1/DMSXL mice by treatment with anti-TfR1 Fab-ASO conjugate. Composite splicing index data shown in FIG. 5 were based on Bin1 E11, Insr E11, Ldb3 E11 and Nfix E7 splicing data; Dtna E12, Mbnl2 E5, Mbnl2 E6, and Ttn E313 were not included because their normalized PSI values in diaphragm tissue were not changed in hTfR1/DMSXL mice relative to hTfR1 mice under the experimental conditions tested.

FIG. 6 shows that anti-TfR1 Fab-ASO conjugate corrected splicing in tibialis anterior tissue of hTfR1/DMSXL mice, as demonstrated by composite splicing index data. The normalized PSI values used to generate the composite splicing index data showed correction of Bin1 E11, Ldb3 E11, and Nfix E7 splicing in tibialis anterior tissue of hTfR1/DMSXL mice by treatment with anti-TfR1 Fab-ASO conjugate, but did not show correction of Mbnl2 E6 splicing. Composite splicing index data shown in FIG. 6 were based on Bin1 E11, Ldb3 E11, Mbnl2 E6, and Nfix E7 splicing data; Dtna E12, Insr E11, Mbnl2 E5, and Ttn E313 were not included because their normalized PSI values in tibialis anterior tissue were not changed in hTfR1/DMSXL mice relative to hTfR1 mice under the experimental conditions tested.

FIG. 7 shows that anti-TfR1 Fab-ASO conjugate corrected splicing in gastrocnemius tissue of hTfR1/DMSXL mice, as demonstrated by composite splicing index data. The normalized PSI values used to generate the composite splicing index data showed correction of Mbnl2 E6, Nfix E7, and Ttn E313 splicing in gastrocnemius tissue of hTfR1/DMSXL mice by treatment with anti-TfR1 Fab-ASO conjugate. Composite splicing index data shown in FIG. 7 were based on Mbnl2 E6, Nfix E7, and Ttn E313 splicing data; Bin1 E11, Dtna E12, Insr E11, Ldb3 E11, and Mbnl2 E5 were not included because their normalized PSI values in gastrocnemius tissue were not changed in hTfR1/DMSXL mice relative to hTfR1 mice under the experimental conditions tested.

These results demonstrate that administration of anti-TfR1 Fab-ASO conjugate facilitates correction of DM1 splicing defects in cardiac and skeletal muscle.

### Example 4. DMPK Knockdown in Non-Human Primate and DM1 Patient Myotubes

Conjugates (labeled in this Example as “Anti-TfR1 Fab-ASO conjugate”) as described in Example 1, containing an anti-TfR1 Fab covalently linked to a DMPK-targeting oligonucleotide (ASO) were tested in human DM1 patient myotubes (32F cells) and in non-human primate (NHP) myotubes. The DM1 patient myotubes used express both a mutant DMPK mRNA containing 380 CUG repeats and a wild-type DMPK mRNA. The NHP myotubes used express only wild-type DMPK.

DM1 patient cells or NHP cells were seeded at a density of 50,000 cells per well in 96 well plates in growth medium and were allowed to recover overnight. The following day, the growth medium was changed to a low-serum differentiation medium and the cells were treated with conjugates at a concentration equivalent to 125 nM, 250 nM, or 500 nM ASO. The cells were incubated for ten days, then cDNA was synthesized using the Cells-to-Ct kit with crude cell lysates as the source of total RNA.

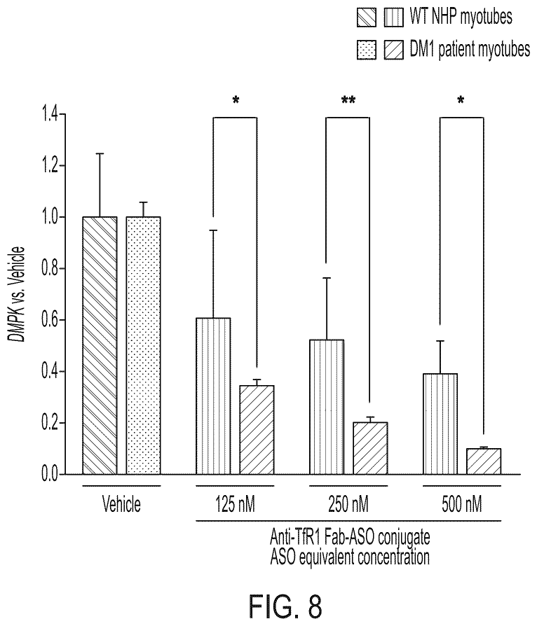

cDNA was used to assess total DMPK knockdown using Taqman PCR. The data was normalized to PPIB expression and the 2^{-ΔΔCt} method was used to determine DMPK knock down compared to a PBS-treated control (“Vehicle”). Data shown in FIG. 8 are presented as mean DMPK expression relative to species-matched vehicle control+standard deviation (n=4 replicates per condition).

The results show that the anti-TfR1 Fab-ASO conjugates achieved knockdown of DMPK expression in both normal NHP myotubes and DM1 patient myotubes, with greater knockdown of DMPK expression in DM1 patient cells (expressing both DMPK mRNA containing 380 CUG repeats and wild-type DMPK mRNA) compared to NHP cells (expressing only wild-type DMPK mRNA) when treated at physiologically relevant concentrations (FIG. 8). At an ASO-equivalent concentration of 125 nM, the conjugates achieved approximately 40% DMPK knockdown relative to vehicle-only control in NHP myotubes, and approximately 65% DMPK knockdown in DM1 patient myotubes. At an ASO-equivalent concentration of 250 nM, the conjugates achieved approximately 45% DMPK knockdown relative to vehicle-only control in NHP myotubes, and approximately 80% DMPK knockdown in DM1 patient myotubes. At an ASO-equivalent concentration of 500 nM, the conjugates achieved approximately 60% DMPK knockdown relative to vehicle-only control in NHP myotubes, and approximately 90% DMPK knockdown in DM1 patient myotubes.

These results indicate that conjugates containing anti-TfR1 Fab covalently linked to a DMPK-targeting oligonucleotide can achieve greater knockdown of DMPK in human myotubes expressing both wild-type DMPK mRNA and mutant DMPK mRNA (with expanded CUG repeats) relative to cynomolgus monkey myotubes expressing wild-type DMPK.

### Example 5. Comparison of Thermal Stability of Different Exemplary Formulations

Formulations comprising conjugates as described in Example 1, containing an anti-TfR1 Fab covalently linked to a DMPK-targeting oligonucleotide (ASO) were prepared. The anti-TfR1 Fab-oligonucleotide conjugates starting material (“SM”) was at a concentration of 33 mg/mL in a buffer containing 100 mM sodium phosphate and 100 mM sodium chloride at pH 7.4. For stability testing, the anti-TfR1 Fab-oligonucleotide conjugates were formulated in different buffers at a concentration of approximately 40 mg/mL. The buffer conditions in each of the four formulations were as follows:
Formulation buffer 1:25 mM succinate, 10% (w/v) sucrose, pH 5.2.
Formulation buffer 2:25 mM succinate, 10% (w/v) sucrose, pH 6.0.
Formulation buffer 3:25 mM tris(hydroxymethyl)aminomethane, 10% (w/v) sucrose, pH 7.5.
Formulation buffer 4:100 mM sodium phosphate, 100 mM sodium chloride, pH 7.4.

The formulation drug substance (formulation with conjugates described herein) was buffer exchanged into formulation buffers 1-4 as provided above using Vivaspin® 6 centrifugal membrane filters (30 kDa molecular weight cutoff). Following buffer exchange, the anti-TfR1 Fab-oligonucleotide conjugates were concentrated to approximately 40 mg/ml. Aliquots of approximately 150 μL from each of the concentrated samples were used to study thermal stability of the conjugates in each formulation. The samples were incubated for 1 week (“T1”), 2 weeks (“T2”), 4 weeks (“T4”), or 8 weeks (“T8”) at −20° C., 2-8° C., 25° C., or 40° C. in glass vials. No change in color, appearance, or pH was observed in any of the test samples at any temperature or time point tested.

The thermal stability of each formulation was first tested by measuring the concentration of the conjugates in the various samples at T0 (week 0), T4 and T8, and at the specified temperatures. Standard BCA (Bicinchoninic Acid) analysis was used to measure the concentration. BCA assay was carried out using standard procedure:
1) Working Reagent (WR) was prepared by thoroughly mixing 20 mL of BCA reagent A with 200 μL BCA reagent B.
2) A standard curve and dilution curves for the test samples were prepared. The test samples were first diluted 1:4, then 20 μL of each test sample or standard was thoroughly mixed with 200 μL of WR and incubated at 60° C. for 10 minutes. The resulting solution was added to the first row of a 96-well plate and serially diluted. A 6-point standard curve was prepared.
3) Absorbance at 480 nm was measured on a plate reader.
4) The standard curve was used to determine the protein concentration of each test sample. Five dilutions were measured for each test sample.

The results of the concentration measurements of the thermal stability study are shown in Table 3 below.

TABLE 3

| | | Measured average concentration of conjugates (mg/mL)^{†} | | | |
|---|---|---|---|---|---|
| Time | Temp. | Formulation | Formulation | Formulation | Formulation |
| point | (° C.) | 1 | 2 | 3 | 4 |
| T0 | N/A | 36.90 | 42.44 | 44.28 | 44.29 |
| T4 | −20 | 32.16 | 43.72 | 43.50 | 43.28 |
| | 2-8 | 31.70 | 42.62 | 39.31 | 40.42 |
| | 25 | 34.52 | 40.02 | 42.69 | 41.94 |
| | 40 | 41.00 | 41.40 | 42.60 | 42.03 |
| T8 | −20 | 32.43 | 42.15 | 42.01 | 42.67 |
| | 2-8 | 32.83 | 41.18 | 41.49 | 42.09 |
| | 25 | 34.23 | 43.80 | 42.16 | 43.15 |
| | 40 | 42.90 | 45.40 | 42.37 | 43.32 |

^{†}Concentrations shown were calculated from the average of measurements of 5 serial dilutions of each test sample.

The results in Table 3 show that the concentrations of the Fab-oligonucleotide conjugates in Formulations 2, 3, and 4 are consistent at T0, T4, and T8. The concentration of the Fab-oligonucleotide conjugates in Formulation 1 at T4 and T8 showed reduced concentration at the lower storage temperatures compared to the T0 measurement.

Thermal stability of the Fab-oligonucleotide conjugates was further studied by measurement of aggregate formation at a concentration of 40 mg/mL. After dilution to 1 mg/mL, T0, T1, T2, T4, and T8 samples incubated at various specified temperatures were the analyzed by size exclusion chromatography (SEC-UV) using a Waters ACQUITY UPLC Protein BEH SEC Column (125 Å, 1.7 μm, 4.6×150 mm) with a mobile phase of 0.2 M potassium phosphate pH 6.8, 0.2 M potassium chloride isocratic elution, 15% isopropyl alcohol. The column temperature was 30% C, and 10 g injections were used. The flow rate was 0.35 mL/min, and analysis was conducted at a wavelength of 280 nm.

Percentages of high molecular weight (HMW) species, Fab-oligonucleotide conjugate monomers, and low molecular weight (LMW) species were measured in the SEC thermal stability analysis for each sample. The results are shown in Table 4 below. The values for the starting material (SM) are shown in the top row for comparison.

TABLE 4

| | | | | |
|---|---|---|---|---|
| Time | Temp. | % HMW | % | % LMW |
| point | (° C.) | species | monomer | species |

| | | | |
|---|---|---|---|
| SM | 10.0 | 89.9 | 0.2 |

Formulation 1

| | | | | |
|---|---|---|---|---|
| T0 | N/A | 7.6 | 92.0 | 0.4 |
| T1 | −20 | 5.7 | 93.6 | 0.7 |
| | 2-8 | 4.4 | 94.8 | 0.8 |
| | 25 | 4.8 | 94.0 | 1.2 |
| | 40 | 4.4 | 94.0 | 1.6 |
| T2 | −20 | 7.5 | 91.8 | 0.7 |
| | 2-8 | 7.5 | 91.8 | 0.7 |
| | 25 | 7.5 | 90.5 | 2.0 |
| | 40 | 6.6 | 91.2 | 2.1 |
| T4 | −20 | 7.5 | 92.4 | 0.1 |
| | 2-8 | 8.1 | 91.5 | 0.5 |
| | 25 | 7.2 | 90.7 | 2.1 |
| | 40 | 6.6 | 88.3 | 5.1 |
| T8 | −20 | 8.8 | 90.7 | 0.5 |
| | 2-8 | 8.9 | 90.3 | 0.8 |
| | 25 | 8.6 | 89.2 | 2.2 |
| | 40 | 8.3 | 85.8 | 5.9 |

Formulation 2

| | | | | |
|---|---|---|---|---|
| T0 | N/A | 10.0 | 89.7 | 0.2 |
| T1 | −20 | 9.8 | 90.0 | 0.2 |
| | 2-8 | 9.9 | 89.9 | 0.2 |
| | 25 | 9.3 | 90.2 | 0.6 |
| | 40 | 8.6 | 89.9 | 1.5 |
| T2 | −20 | 9.3 | 90.4 | 0.3 |
| | 2-8 | 9.4 | 90.2 | 0.4 |
| | 25 | 9.1 | 90.0 | 0.9 |
| | 40 | 8.4 | 89.2 | 2.3 |
| T4 | −20 | 9.1 | 90.8 | 0.1 |
| | 2-8 | 9.4 | 90.4 | 0.2 |
| | 25 | 8.3 | 90.6 | 1.1 |
| | 40 | 7.9 | 89.7 | 2.4 |
| T8 | −20 | 9.5 | 90.3 | 0.2 |
| | 2-8 | 9.6 | 89.9 | 0.5 |
| | 25 | 8.7 | 89.4 | 1.9 |
| | 40 | 8.8 | 87.0 | 4.2 |

Formulation 3

| | | | | |
|---|---|---|---|---|
| T0 | N/A | 9.8 | 89.8 | 0.5 |
| T1 | −20 | 10.1 | 89.6 | 0.3 |
| | 2-8 | 9.8 | 89.8 | 0.4 |
| | 25 | 9.2 | 89.8 | 1.0 |
| | 40 | 8.7 | 89.6 | 1.7 |
| T2 | −20 | 9.5 | 90.1 | 0.4 |
| | 2-8 | 9.3 | 90.1 | 0.6 |
| | 25 | 8.5 | 90.2 | 1.3 |
| | 40 | 8.8 | 89.2 | 2.0 |
| T4 | −20 | 8.7 | 91.1 | 0.2 |
| | 2-8 | 7.8 | 90.9 | 1.3 |
| | 25 | 7.9 | 90.8 | 1.3 |
| | 40 | 8.8 | 88.7 | 2.5 |
| T8 | −20 | 8.9 | 90.6 | 0.5 |
| | 2-8 | 8.5 | 90.7 | 0.8 |
| | 25 | 8.6 | 89.8 | 1.6 |
| | 40 | 11.0 | 86.0 | 3.0 |

Formulation 4

| | | | | |
|---|---|---|---|---|
| T0 | N/A | 9.6 | 90.2 | 0.2 |
| T1 | −20 | 9.1 | 90.8 | 0.2 |
| | 2-8 | 9.7 | 90.1 | 0.2 |
| | 25 | 9.2 | 90.2 | 0.7 |
| | 40 | 9.2 | 88.9 | 1.9 |
| T2 | −20 | 9.5 | 90.2 | 0.3 |
| | 2-8 | 9.6 | 90.0 | 0.4 |
| | 25 | 9.6 | 89.3 | 1.2 |
| | 40 | 9.2 | 88.4 | 2.5 |
| T4 | −20 | 8.5 | 91.4 | 0.1 |
| | 2-8 | 8.7 | 91.0 | 0.2 |
| | 25 | 8.9 | 89.8 | 1.3 |
| | 40 | 9.0 | 87.4 | 3.6 |
| T8 | −20 | 9.7 | 90.0 | 0.3 |
| | 2-8 | 9.7 | 89.8 | 0.5 |
| | 25 | 9.9 | 88.3 | 1.8 |
| | 40 | 11.1 | 83.8 | 5.1 |

The results showed no significant change in percent monomer with increasing temperature or over time, and a slight increase in LMW species was observed over time with increasing temperature.

Overall, the SEC analysis indicated that Formulations 1, 2, and 3 provided slightly improved stability with respect to % monomer at the 8-week time point, compared to formulation 4. Formulation 3 showed the best stability with respect to changes in % LMW species.

Thermal stability of the Fab-oligonucleotide conjugates at 40 mg/mL was further studied by non-reduced protein express assay using capillary electrophoresis (CE-SDS). For analysis by this method, samples were diluted to 1 mg/mL, then mixed 1:1 with 10 mM N-ethylmaleimide in PBS. 5 μL of each of these samples was then added to 7 μL of sample buffer (Protein Express Reagent Kit, Perkin Elmer CLS960008) in a 96-well plate. Samples were incubated at 70° C. for 10 minutes, then cooled on ice for 5 minutes. 32 μL of water was added to each sample well, and the plate was centrifuged at 1200 g for 2 minutes prior to analysis. Samples were analyzed on an HT Protein Express LabChip (Perkin Elmer 760499), using a LabChip GXII Touch HT (Perkin Elmer), using measurement method HT High Sensitivity Antibody Analysis 200.

Results of the non-reduced CE-SDS analysis are shown in Table 5 below. For comparison, the starting material showed a size of 62.4 kDa with 100.0% purity.

TABLE 5

| | | Size^{†} and % purity | | | | | | | |
|---|---|---|---|---|---|---|---|---|---|
| Time point | Temp.(° C.) | Formulation 1 | | Formulation 2 | | Formulation 3 | | Formulation 4 | |
| T0 | N/A | 65.0 | (100.0) | 63.5 | (100.0) | 63.3 | (100.0) | 62.6 | (100.0) |
| T1 | −20 | 66.0 | (100.0) | 64.3 | (100.0) | 65.1 | (100.0) | 64.2 | (100.0) |
| | 2-8 | 66.2 | (100.0) | 65.4 | (100.0) | 64.0 | (100.0) | 63.7 | (100.0) |
| | 25 | 66.3 | (98.4) | 64.1 | (98.9) | 64.1 | (98.8) | 63.3 | (98.7) |
| | 40 | 65.1 | (97.4) | 63.1 | (97.8) | 62.9 | (97.9) | 63.2 | (96.8) |
| T2 | −20 | 66.0 | (100.0) | 64.6 | (100.0) | 63.5 | (100.0) | 63.2 | (100.0) |
| | 2-8 | 66.4 | (100.0) | 64.6 | (100.0) | 63.0 | (100.0) | 63.3 | (100.0) |
| | 25 | 66.1 | (97.7) | 64.0 | (98.8) | 63.3 | (98.8) | 63.4 | (98.5) |
| | 40 | 66.3 | (95.7) | 64.3 | (97.5) | 63.3 | (97.9) | 63.5 | (96.7) |
| T4 | −20 | 66.6 | (100.0) | 63.7 | (100.0) | 63.5 | (100.0) | 64.9 | (100.0) |
| | 2-8 | 67.0 | (100.0) | 63.7 | (100.0) | 63.2 | (100.0) | 64.0 | (100.0) |
| | 25 | 66.2 | (97.0) | 63.5 | (98.5) | 63.8 | (98.6) | 64.7 | (98.2) |
| | 40 | 66.0 | (84.1) | 64.1 | (96.6) | 63.7 | (97.1) | 64.3 | (95.9) |
| T8 | −20 | 66.4 | (100.0) | 65.9 | (100.0) | 65.4 | (100.0) | 64.2 | (100.0) |
| | 2-8 | 66.7 | (100.0) | 65.9 | (100.0) | 65.7 | (100.0) | 64.2 | (100.0) |
| | 25 | 67.1 | (96.9) | 64.6 | (98.8) | 64.9 | (98.2) | 63.7 | (97.7) |
| | 40 | 67.4 | (74.8) | 65.9 | (95.0) | 64.7 | (96.4) | 64.1 | (94.9) |

^{†}Size is shown in kDa. Values in parentheses represent the percent purity.

The results showed that the percent purity of conjugates in Formulation 1 gradually decreased at 25° C. and 40° C. from T1 to T8, and the formation of new species was observed, suggesting that conjugates in Formulation 1 are less stable at higher temperatures. The results also showed that conjugates had good percent purity in Formulations 2, 3, and 4 at all temperatures and time points tested. The high percent purity of conjugates in Formulation 3 at higher temperatures suggests that they have improved stability in Formulation 3 relative to the other formulations.

Thermal stability of the Fab-oligonucleotide conjugates at 40 mg/mL was further studied by reduced protein express assay using capillary electrophoresis (CE-SDS). For analysis by this method, samples were diluted with PBS to 0.5 mg/mL. Samples were next mixed with denaturing solution: 24.5 μL of 14.2 M P3-mercaptoethanol (BME) was added to 700 μL of Protein Express Sample Buffer (Protein Express Reagent Kit, Perkin Elmer CLS960008), and then 5 μL of each of the diluted samples was added to 7 μL of the sample denaturing solution in a 96-well plate. Samples were incubated at 70° C. for 10 minutes, then cooled on ice for 5 minutes. 32 μL of water was added to each sample well, and the plate was centrifuged at 1200 g for 2 minutes prior to analysis. Samples were analyzed on an HT Protein Express LabChip (Perkin Elmer 760499), using a LabChip GXII Touch HT (Perkin Elmer), using measurement method HT High Sensitivity Antibody Analysis 200.

Results of the reduced CE-SDS analysis are shown in Table 6 below. Values for the starting material (SM) are shown for comparison in the top row.

TABLE 6

| | | | | | | | |
|---|---|---|---|---|---|---|---|
| | | LC | | Fab H/L | % Fab | Other | |
| Time | Temp. | Fragment | % LC | Fragment | H/L | Fragment | % Other |
| point | (° C.) | Size^{†} | Fragment | Size^{†} | Fragment | Size^{†} | Fragment |

| | | | | | | |
|---|---|---|---|---|---|---|
| SM | 28.4 | 3.0 | 34.9 | 87.3 | 41.7 | 9.7 |

Formulation 1

| | | | | | | | |
|---|---|---|---|---|---|---|---|
| T0 | N/A | 27.7 | 15.3 | 35.6 | 79.9 | 41.7 | 5.2 |
| T1 | −20 | 27.0 | 3.9 | 34.2 | 89.8 | 41.0 | 6.4 |
| | 2-8 | 26.9 | 3.9 | 33.8 | 89.9 | 40.7 | 6.3 |
| | 25 | 27.8 | 4.0 | 34.9 | 89.7 | 42.0 | 6.3 |
| | 40 | 27.8 | 4.4 | 34.9 | 89.0 | 42.1 | 6.6 |
| T2 | −20 | 27.6 | 3.9 | 34.4 | 44.9 | 42.4 | 5.6 |
| | 2-8 | 27.4 | 4.0 | 34.1 | 45.8 | 42.0 | 5.6 |
| | 25 | 27.5 | 4.3 | 34.0 | 46.7 | 42.1 | 5.3 |
| | 40 | 27.4 | 4.9 | 33.9 | 45.3 | 41.8 | 5.2 |
| T4 | −20 | 27.1 | 3.7 | 33.7 | 48.0 | 41.5 | 6.2 |
| | 2-8 | 27.2 | 3.8 | 33.8 | 46.9 | 41.9 | 6.1 |
| | 25 | 26.9 | 4.4 | 33.4 | 47.2 | 41.3 | 5.4 |
| | 40 | 27.3 | 5.6 | 33.7 | 50.0 | 41.6 | 4.5 |
| T8 | −20 | 27.3 | 3.1 | 34.3 | 48.1 | 41.8 | 5.0 |
| | 2-8 | 27.6 | 3.4 | 34.3 | 46.1 | 42.2 | 5.1 |
| | 25 | 27.3 | 3.8 | 34.0 | 47.1 | 41.7 | 4.8 |
| | 40 | 27.5 | 6.4 | 33.9 | 53.9 | 41.7 | 4.3 |

Formulation 2

| | | | | | | | |
|---|---|---|---|---|---|---|---|
| T0 | N/A | 28.2 | 3.0 | 34.8 | 87.5 | 41.4 | 9.4 |
| T1 | −20 | 27.3 | 3.8 | 33.8 | 87.0 | 40.9 | 9.2 |
| | 2-8 | 27.1 | 3.7 | 33.5 | 86.5 | 40.6 | 8.8 |
| | 25 | 27.6 | 4.0 | 34.4 | 87.5 | 41.8 | 8.4 |
| | 40 | 28.1 | 4.3 | 34.8 | 87.4 | 42.3 | 8.3 |
| T2 | −20 | 27.5 | 4.5 | 34.3 | 87.5 | 42.0 | 8.0 |
| | 2-8 | 27.4 | 4.2 | 34.1 | 88.2 | 41.8 | 7.6 |
| | 25 | 26.7 | 4.7 | 33.2 | 87.0 | 40.9 | 7.3 |
| | 40 | 26.8 | 5.3 | 33.3 | 87.7 | 40.9 | 7.0 |
| T4 | −20 | 27.1 | 4.3 | 33.8 | 87.7 | 41.3 | 8.0 |
| | 2-8 | 27.0 | 4.2 | 33.7 | 88.2 | 41.2 | 7.6 |
| | 25 | 27.4 | 4.7 | 34.1 | 86.9 | 41.8 | 7.2 |
| | 40 | 27.0 | 5.1 | 33.6 | 85.8 | 41.1 | 6.6 |
| T8 | −20 | 27.5 | 3.7 | 34.3 | 89.1 | 41.8 | 7.2 |
| | 2-8 | 27.2 | 3.8 | 34.0 | 89.3 | 41.5 | 6.9 |
| | 25 | 27.1 | 6.3 | 33.7 | 87.7 | 41.1 | 6.0 |
| | 40 | 27.2 | 4.6 | 34.0 | 88.7 | 41.4 | 6.7 |

Formulation 3

| | | | | | | | |
|---|---|---|---|---|---|---|---|
| T0 | N/A | 28.3 | 3.1 | 34.8 | 87.6 | 41.4 | 9.4 |
| T1 | −20 | 27.2 | 3.8 | 33.7 | 87.0 | 41.1 | 9.2 |
| | 2-8 | 27.3 | 4.0 | 33.8 | 86.9 | 41.1 | 9.2 |
| | 25 | 28.0 | 4.1 | 34.7 | 87.0 | 42.2 | 8.8 |
| | 40 | 28.0 | 4.5 | 34.7 | 87.3 | 42.0 | 8.2 |
| T2 | −20 | 26.8 | 4.6 | 33.4 | 87.5 | 40.9 | 8.0 |
| | 2-8 | 27.2 | 4.6 | 33.8 | 87.5 | 41.4 | 7.9 |
| | 25 | 26.6 | 5.1 | 33.2 | 86.3 | 40.6 | 7.7 |
| | 40 | 26.8 | 5.5 | 33.2 | 82.4 | 40.6 | 6.1 |
| T4 | −20 | 26.9 | 4.4 | 33.5 | 87.5 | 41.0 | 8.1 |
| | 2-8 | 26.8 | 4.9 | 33.4 | 86.8 | 40.9 | 8.3 |
| | 25 | 27.2 | 4.9 | 33.8 | 86.7 | 41.4 | 7.4 |
| | 40 | 27.3 | 6.0 | 33.9 | 84.6 | 41.5 | 6.8 |
| T8 | −20 | 26.9 | 4.2 | 33.5 | 88.0 | 41.0 | 7.8 |
| | 2-8 | 27.0 | 4.3 | 33.6 | 88.4 | 41.0 | 7.3 |
| | 25 | 27.0 | 5.1 | 33.7 | 88.5 | 41.1 | 7.0 |
| | 40 | 27.2 | 6.3 | 33.8 | 84.3 | 41.3 | 5.7 |

Formulation 4

| | | | | | | | |
|---|---|---|---|---|---|---|---|
| T0 | N/A | 28.0 | 3.0 | 34.5 | 87.6 | 41.1 | 9.4 |
| T1 | −20 | 27.1 | 3.7 | 33.6 | 87.6 | 40.9 | 8.7 |
| | 2-8 | 27.1 | 3.8 | 33.7 | 87.4 | 40.9 | 8.8 |
| | 25 | 27.6 | 3.9 | 34.2 | 87.4 | 41.6 | 8.7 |
| | 40 | 27.4 | 4.9 | 34.1 | 87.0 | 41.5 | 8.1 |
| T2 | −20 | 27.0 | 4.3 | 33.6 | 87.3 | 41.2 | 8.1 |
| | 2-8 | 27.4 | 4.4 | 33.9 | 87.6 | 41.4 | 8.0 |
| | 25 | 27.0 | 4.7 | 33.7 | 87.8 | 41.2 | 7.5 |
| | 40 | 26.8 | 5.9 | 33.5 | 86.9 | 40.8 | 7.2 |
| T4 | −20 | 27.2 | 3.8 | 33.8 | 89.1 | 41.4 | 7.1 |
| | 2-8 | 26.7 | 4.3 | 33.8 | 88.0 | 40.6 | 7.7 |
| | 25 | 27.1 | 4.9 | 33.7 | 87.8 | 41.3 | 7.3 |
| | 40 | 27.2 | 6.3 | 33.8 | 86.8 | 41.4 | 6.7 |
| T8 | −20 | 26.9 | 4.2 | 33.5 | 87.7 | 40.9 | 8.1 |
| | 2-8 | 26.8 | 4.1 | 33.5 | 88.6 | 40.8 | 7.3 |
| | 25 | 26.7 | 5.1 | 33.4 | 88.0 | 40.7 | 5.4 |
| | 40 | 26.8 | 6.8 | 33.4 | 87.7 | 40.7 | 5.4 |

^{†}Sizes are shown in kDa.

In Formulation 1, there were two additional peaks including one additional HC peak observed in all samples at T2, T4, and T8, indicating instability of the conjugates in this formulation. There were no significant differences in overall stability of the conjugates in formulations 2, 3, and 4 observed by the reduced CE-SDS.

Overall, the results of the various thermal stability tests presented herein suggest that Formulation 3 provided the best stability for the Fab-oligonucleotide conjugates.

### Example 6. Comparison of Freeze/Thaw Stability of Different Exemplary Formulations

Formulations comprising conjugates as described in Example 1, containing an anti-TfR1 Fab covalently linked to a DMPK-targeting oligonucleotide (ASO) were prepared. The anti-TfR1 Fab-oligonucleotide conjugates starting material (“SM”) was at a concentration of 33 mg/mL in a buffer containing 100 mM sodium phosphate and 100 mM sodium chloride at pH 7.4. For freeze/thaw stability testing, the anti-TfR1 Fab-oligonucleotide conjugates were formulated in different buffers at a concentration of approximately 40 mg/mL. The buffer conditions in each of the four formulations were as follows:
Formulation buffer 1:25 mM succinate, 10% (w/v) sucrose, pH 5.2.
Formulation buffer 2:25 mM succinate, 10% (w/v) sucrose, pH 6.0.
Formulation buffer 3:25 mM tris(hydroxymethyl)aminomethane, 10% (w/v) sucrose, pH 7.5.
Formulation buffer 4:100 mM sodium phosphate, 100 mM sodium chloride, pH 7.4.

To test freeze/thaw stability of the conjugates in each formulation, aliquots were prepared and subjected to 0, 1, 3, or 5 freeze/thaw cycles, after which time visual appearance, protein concentration, and aggregate/fragment formation were assessed.

For each of formulations 1, 2, 3, and 4, visual appearance assessment showed the solutions were clear, colorless, and with no visible aggregates after 0, 1, 3, and 5 freeze/thaw cycles.

BCA analysis of each formulation, conducted as described in Example 5, suggested that there were no significant changes in the concentration of the complexes over 5 freeze/thaw (“F/T”) cycles. The results are shown in Table 7 below. Values for the starting material (SM) are shown for comparison in the top row.

TABLE 7

| | Concentration (mg/mL) | | | |
|---|---|---|---|---|
| F/T | Formulation | Formulation | Formulation | Formulation |
| cycles | 1 | 2 | 3 | 4 |
| 0 | 34.4 | 42.4 | 40.6 | 44.2 |
| 1 | 35.5 | 42.3 | 41.9 | 44.7 |
| 3 | 35.4 | 42.1 | 43.3 | 43.2 |
| 5 | 36.4 | 42.5 | 42.9 | 44.1 |

Size exclusion chromatography (SEC) analysis of each formulation, conducted as described in Example 5, suggested that there were no significant differences between the four formulations with respect to low molecular weight (LMW) species, monomer, and high molecular weight (HMW) species over 5 freeze/thaw (“F/T”) cycles. The results are shown in Table 8 below. Values for the starting material (SM) are shown for comparison in the top row.

TABLE 8

| F/T cycles | % HMW species | % monomer | % LMW species |
|---|---|---|---|
| SM | 9.48 | 89.70 | 0.83 |

Formulation 1

| | | | |
|---|---|---|---|
| 0 | 8.15 | 89.99 | 1.86 |
| 1 | 7.68 | 91.20 | 1.11 |
| 3 | 7.62 | 90.83 | 1.55 |
| 5 | 7.68 | 91.08 | 1.25 |

Formulation 2

| | | | |
|---|---|---|---|
| 0 | 9.50 | 89.66 | 0.84 |
| 1 | 9.45 | 89.71 | 0.84 |
| 3 | 9.49 | 89.68 | 0.82 |
| 5 | 9.55 | 89.67 | 0.78 |

Formulation 3

| | | | |
|---|---|---|---|
| 0 | 8.61 | 90.03 | 1.36 |
| 1 | 8.73 | 89.81 | 1.46 |
| 3 | 8.76 | 89.74 | 1.50 |
| 5 | 8.77 | 89.77 | 1.46 |

Formulation 4

| | | | |
|---|---|---|---|
| 0 | 9.73 | 89.24 | 1.03 |
| 1 | 9.63 | 89.46 | 0.92 |
| 3 | 9.56 | 89.45 | 1.00 |
| 5 | 9.48 | 89.52 | 0.99 |

### Example 7. Evaluation of Stability of Lyophilized Formulations of Conjugates

Conjugates (labeled in this Example as “Anti-TfR1 Fab-ASO conjugate”) as described in Example 1, containing an anti-TfR1 Fab covalently linked to a DMPK-targeting oligonucleotide (ASO) were prepared and lyophilized with tris(hydroxymethyl)aminomethane (25 mM), sucrose (10% (w/v)), at a pH of 7.5 and tested for stability. Prior to reconstitution all samples at all timepoints and temperatures were visually identified as a white cake and uniform in shape. Additionally, the samples reconstituted in less than 1 minute, and became clear and colorless with fewer than 10 particles. All samples were considered stable by visual analysis pre- and post-reconstitution up to 400 for at least 8 weeks.

X-ray diffraction analysis was performed to evaluate the crystallographic structure of the lyophilized composition. Approximately 20 mg of lyophilized anti-TfR1 Fab-ASO conjugates was placed in a plastic sample holder and flattened with a glass slide to ensure a flat surface. When performing this analysis, a Proto AXRD® Benchtop Power X-ray Diffractometer was used with a fine focus Cu-anode metal/ceramic construction 1500-Watt x-ray tube.

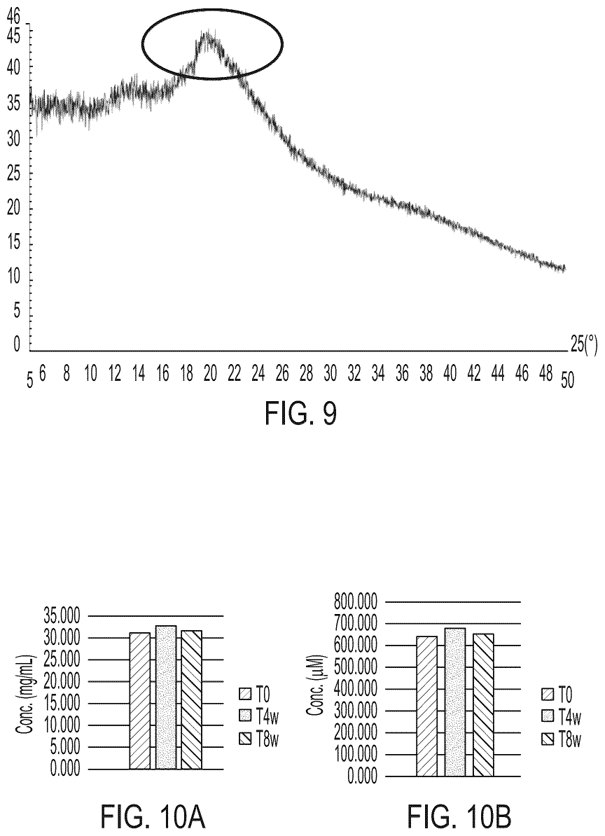

The diffraction pattern was measured (FIG. 9), and a broad “halo” (circled in FIG. 9) was identified, which indicates that the material is amorphous at time 0 (T0).

To test the absorbance of the samples, the lyophilized anti-TfR1 Fab-ASO conjugates were first reconstituted with water and then diluted using formulation buffer (25 mM tris(hydroxymethyl)aminomethane, 10% (w/v) sucrose, pH 7.5) to a concentration of 0.2 mg/ml. Absorbance was measured at 260 nm and 280 nm using a DeNovix DS11+ spectrophotometer. Readings were performed in triplicate and averaged. These results were used to calculate the concentration (Fab (mg/mL and μM) and oligonucleotide (μM)), percent recovery, and Drug Antibody Ratio (DAR), at multiple timepoints. For all protein concentrations, percent recovery, and DAR testing, bovine serum albumin (BSA) was used as a positive control, PBS was used as a blank and negative control for BSA, and formulation buffer was used as a blank and negative control for the conjugates samples.

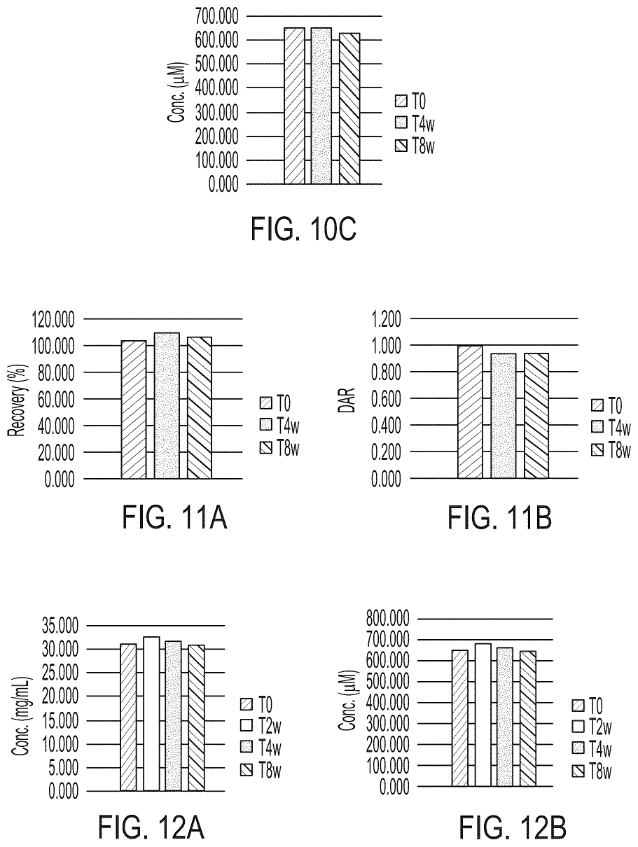

At 2-8° C. the average concentration of the replicates for Fab (FIG. 10A and FIG. 10B) showed no significant change from time 0 (T0), when compared to 4 weeks (T4w) or 8 weeks (T8w). Additionally, there was no significant change when comparing the 4 week and 8-week timepoints. At 2-8° C. the average concentration of the replicates for oligonucleotide concentration (FIG. 10C) showed no significant difference when comparing T0, T4w, and T8w. At 2-8° C. the average percent recovery (FIG. 11A) was close to 100% for all samples at all time points. At 2-8° C. the average DAR (FIG. 11B) was close to 1.0 at all timepoints.

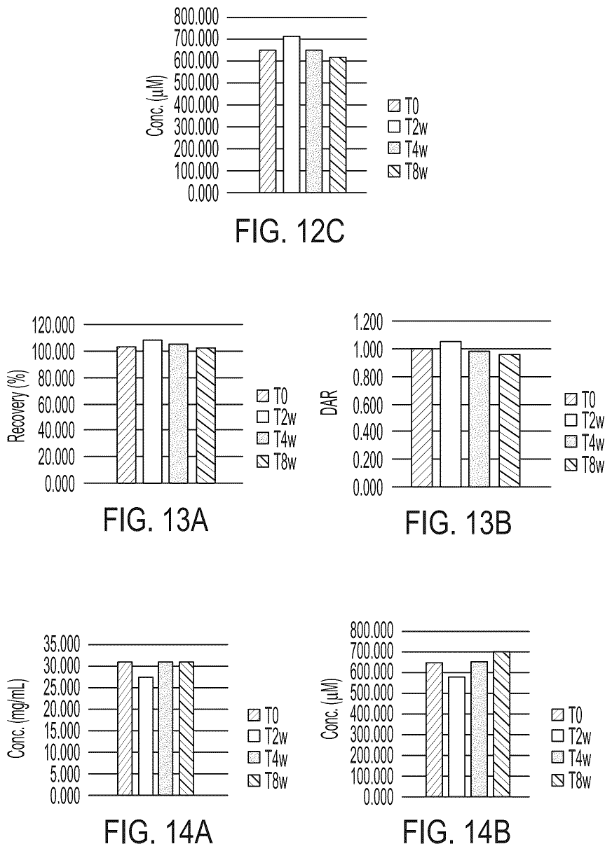

At 25° C. the average concentration of the replicates for Fab (FIG. 12A and FIG. 12B) showed no significant change from time 0 (T0), when compared to 2 weeks (T2w), 4 weeks (T4w), or 8 weeks (T8w) and there was no significant change when comparing T2w, T4w, and T8w to one another. At 25° C. the average concentration of the replicates for the oligonucleotide (FIG. 12C) did not show significant change over time. At 25° C. the average percent recovery (FIG. 13A) was close to 100% for all timepoints. At 25° C. the average DAR (FIG. 13B) was close to 1.0 at all timepoints.

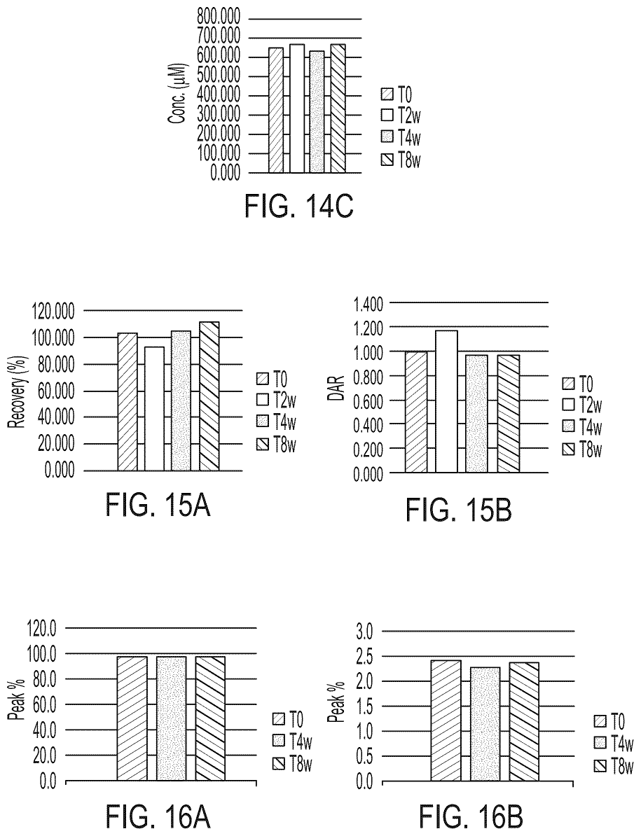

At 40° C. the average concentration of the replicates for Fab (FIG. 14A and FIG. 14B) showed no significant change from T0, when compared to T2w, T4w, or T8w. At 40° C. the average concentration of the replicates for oligonucleotide concentration (FIG. 10C) showed no significant change at any of the timepoints. At 40° C. the average percent recovery (FIG. 15A) was close to 100% for all samples at all time points. At 40° C. the average DAR (FIG. 15B) was close to 1.0 at all timepoints.

Next, the anti-TfR1 Fab-ASO conjugates were analyzed using size-exclusion chromatograph (SEC). All samples were diluted to a concentration of 1.0 mg/ml using formulation buffer (25 mM tris(hydroxymethyl)aminomethane, 10% (w/v) sucrose, pH 7.5) prior to analysis. For the mobile phase of the chromatographic conditions an isocratic elution was used containing potassium phosphate (200 mM), potassium chloride (200 mM), 5% (v/v) isopropanol (IPA) at a pH of 6.8, at a flow rate of 0.250 ml/min using a 20 minute run time. The wavelength used for measurements was 280 nm. The high-performance liquid chromatography system used was the Dionex Ultimate 3000 UPLC System, with a Waters BEH SEC column (200 Å, 300×4.6 mm, 1.7 μm, P/N 186005226).

At 2-8° C. the sum of the relative area of the first and second (main) peaks (FIG. 16A) show no significant difference when comparing T0, T4w, and T8w. Further, the sum of the relative area of the third and fourth (high molecular weight species (HMWS)) peaks (FIG. 16B) also show no significant difference when comparing T0, T4w, and T8w.

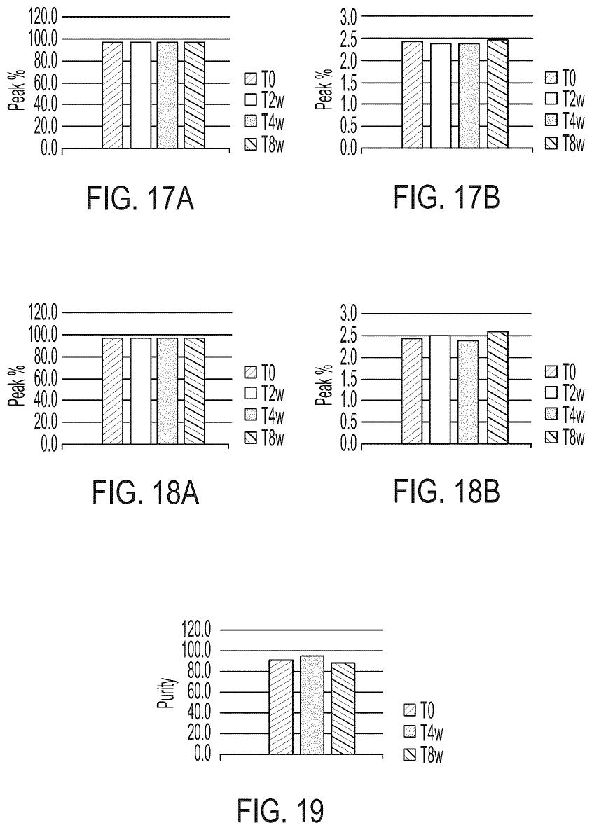

At 25° C. the sum of the relative area of the first and second (main) peaks (FIG. 17A) show no significant difference when comparing T0, T2w, T4w, and T8w. Further, the sum of the relative area of the third and fourth (HMWS) peaks (FIG. 17B) also show no significant difference when comparing T0, T2w, T4w, and T8w.

At 40° C. the sum of the relative area of the first and second (main) peaks (FIG. 18A) show no significant difference when comparing T0, T2w, T4w, and T8w. Further, the sum of the relative area of the third and fourth (HMWS) peaks (FIG. 18B) also show no significant difference when comparing T0, T2w, T4w, and T8w.

To conduct non-reduced capillary electrophoresis sodium dodecyl sulfate (CE-SDS) analysis, samples were diluted to 1 mg/ml in phosphate buffered saline (PBS). The diluted samples were then further diluted 1:1 with 10 mM N-ethylmaleimide (NEM) in PBS. 5.0 μl of the resulting solution was then added to 7.0 μl sample buffer. The samples were then incubated at 70° C. for 10 minutes. After incubation, 32 μl of water was added and the samples were centrifuged at 1200 g for 2 minutes. The samples were then analyzed using HT High Sensitivity Antibody Analysis 200, using HT Protein Express LabChip (Perkin Elmer, 760499) with Protein Express Reagent Kit (Perkin Elmer, CLS960008). Analysis was performed on a LabChip GXII Touch HT (Perkin Elmer).

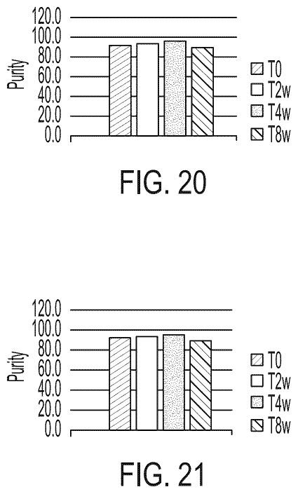

At 2-8° C. (FIG. 19), the purity of the main peak showed no significant change between T0, T4w, and T8w. At 25° C. (FIG. 20) and 40° C. (FIG. 21), the purity of the main peak showed no significant change between T0, T2w, T4w, and T8w.

To conduct reduced CE-SDS analysis, samples were diluted to 0.5 mg/ml in PBS. 5.0 μl of the diluted sample was added to 7.0 μl of sample denaturing solution (24.5 μL of 1M dithiothreitol (DTT) added to 700 μL Protein Express Sample Buffer). The samples were then incubated at 70° C. for 10 minutes. After incubation, 32 μl of water was added and the samples were centrifuged at 1200 g for 2 minutes. The samples were then analyzed using HT High Sensitivity Antibody Analysis 200, using HT Protein Express LabChip (Perkin Elmer, 760499) with Protein Express Reagent Kit (Perkin Elmer, CLS960008). Analysis was performed on a LabChip GXII Touch HT (Perkin Elmer).

At 2-8° C. (Table 9), the purity of the peaks 1, 2, 3, and 4 showed no significant change between T0, T4w, and T8w. At 25° C. (Table 10) and 40° C. (Table 11), the purity of the peaks 1, 2, 3, and 4 showed no significant change between T0, T2w, T4w, and T8w.

Overall, these results demonstrate that lyophilized anti-TfR1 Fab-ASO conjugates are stable at all temperatures and timepoints tested.

TABLE 9
Capillary electrophoresis sodium dodecyl sulfate (CE-SDS)
results (reduced) at 2-8° C.

| Time point | Peak 1 (%) | Peak 2 (%) | Peak 3 (%) | Total (%) |
|---|---|---|---|---|
| T0 | 3.05 | 89.97 | 6.09 | 99.11 |
| T4w | 2.87 | 88.40 | 8.42 | 99.69 |
| T8w | 4.12 | 85.15 | 8.41 | 97.68 |

TABLE 10
Capillary electrophoresis sodium dodecyl sulfate (CE-SDS)
results (reduced) at 25° C.

| Time point | Peak 1 (%) | Peak 2 (%) | Peak 3 (%) | Total (%) |
|---|---|---|---|---|
| T0 | 3.05 | 89.97 | 6.09 | 99.11 |
| T2w | 3.21 | 88.94 | 7.31 | 99.46 |
| T4w | 2.87 | 87.62 | 9.09 | 99.58 |
| T8w | 4.12 | 85.22 | 8.38 | 97.72 |

TABLE 11
Capillary electrophoresis sodium dodecyl sulfate (CE-SDS)
results (reduced) at 40° C.

| Time point | Peak 1 (%) | Peak 2 (%) | Peak 3 (%) | Total (%) |
|---|---|---|---|---|
| T0 | 3.05 | 89.97 | 6.09 | 99.11 |
| T2w | 3.21 | 88.80 | 7.34 | 99.35 |
| T4w | 2.91 | 87.80 | 8.75 | 99.46 |
| T8w | 4.14 | 85.20 | 8.41 | 97.75 |

### ADDITIONAL EMBODIMENTS

1. A formulation comprising complexes that comprise an oligonucleotide covalently linked to an anti-transferrin receptor 1 (TfR1) antibody,
wherein the anti-TfR1 antibody comprises: a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16,
wherein the oligonucleotide comprises a 5′-X—Y—Z-3′ configuration, wherein X and Z are flanking regions comprising one or more modified nucleosides and Y is a gap region comprising one or more 2′-deoxyribonucleosides,
and wherein the complexes are formulated with tris(hydroxymethyl)aminomethane and sucrose.
   2. A formulation comprising complexes comprising a structure of formula (I): [R¹]ₙ₁—R², wherein each R¹ independently comprises a group of the formula (Ia):

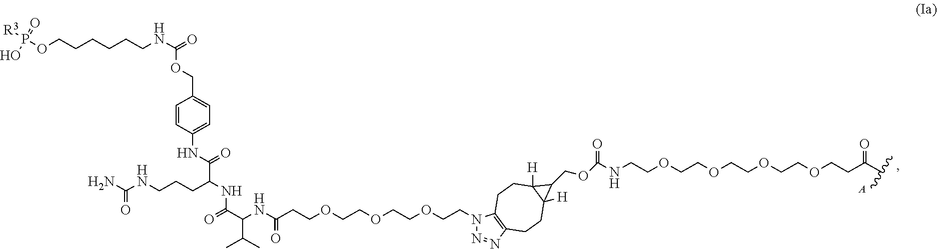

wherein

R² comprises an antibody, and
R³ comprises an oligonucleotide comprising a 5′-X—Y—Z-3′ configuration, wherein X and Z are flanking regions comprising one or more modified nucleosides and Y is a gap region comprising one or more 2′-deoxyribonucleosides;
wherein R¹ is covalently linked to R² at attachment point A; and
wherein n1 is an integer representing the number of instances of R¹, wherein each instance of R¹ is covalently linked to a different amino acid residue of the antibody, optionally wherein each different amino acid residue is a lysine;
wherein the complexes are formulated with tris(hydroxymethyl)aminomethane and sucrose,
optionally wherein the antibody is an anti-TfR1 antibody.
   3. The formulation of embodiment 2, wherein the antibody comprises:
a heavy chain complementarity determining region 1 (CDR-H1) comprising a sequence as set forth in SEQ ID NOs: 1, 7, or 12, a heavy chain complementarity determining region 2 (CDR-H2) comprising a sequence as set forth in SEQ ID NOs: 2, 8, or 13, a heavy chain complementarity determining region 3 (CDR-H3) comprising a sequence as set forth in SEQ ID NOs: 3, 9, or 14, a light chain complementarity determining region 1 (CDR-L1) comprising a sequence as set forth in SEQ ID NOs: 4, 10, or 15, a light chain complementarity determining region 2 (CDR-L2) comprising a sequence as set forth in SEQ ID NOs: 5 or 11, and a light chain complementarity determining region 3 (CDR-L3) comprising a sequence as set forth in SEQ ID NOs: 6 or 16.
   4. The formulation of any one of embodiments 1 to 3, wherein the formulation is in a lyophilized form.
   5. The formulation of any one of embodiments 1 to 3, wherein the formulation is in a frozen solid form.
   6. The formulation of any one of embodiments 1 to 3, wherein the formulation is in an aqueous solution.
   7. The formulation of embodiment 6, wherein the tris(hydroxymethyl)aminomethane is present in the aqueous solution at a concentration in the range of 5 mM to 50 mM.
   8. The formulation of embodiment 6 or 7, wherein the sucrose is present in the aqueous solution at a concentration in the range of 5% to 15% weight per volume (w/v %).
   9. The formulation of any one of embodiments 6 to 8, wherein the aqueous solution has a pH in the range of 6.5 to 8.5.
   10. The formulation of any one of embodiments 6 to 9, wherein the tris(hydroxymethyl)aminomethane is present in the aqueous solution at a concentration of 25 mM and/or the sucrose is present in the aqueous solution at a concentration of 10 w/v % and/or the aqueous solution is at a pH of 7.5.
   11. The formulation of any one of embodiments 1-10, wherein the antibody is a Fab fragment, a full-length IgG, a Fab′ fragment, a F(ab′)₂ fragment, an scFv, or an Fv.
   12. The formulation of embodiment 11, wherein the antibody is a Fab fragment.
   13. The formulation of any one of embodiments 1-12, wherein the antibody comprises a heavy chain variable region (VH) comprising an amino acid sequence at least 85% identical to SEQ ID NO: 17; and/or wherein the antibody comprises a light chain variable region (VL) comprising an amino acid sequence at least 85% identical to SEQ ID NO: 18,
optionally wherein the antibody comprises a VH comprising the amino acid sequence of SEQ ID NO: 17 and/or a VL comprising the amino acid sequence of SEQ ID NO: 18.
   14. The formulation of any one of embodiments 1-13, wherein the antibody comprises a heavy chain comprising an amino acid sequence at least 85% identical to SEQ ID NO: 19; and/or wherein the antibody comprises a light chain comprising an amino acid sequence at least 85% identical to SEQ ID NO: 20,
optionally wherein the antibody comprises a heavy chain comprising the amino acid sequence of SEQ ID NO: 19 and/or a light chain comprising the amino acid sequence of SEQ ID NO: 20.
   15. The formulation of any one of embodiments 1-14, wherein the oligonucleotide is 10-30 nucleotides in length.
   16. The formulation of any one of embodiments 1-15, wherein the oligonucleotide comprises a nucleotide sequence having a region of complementarity of at least 8 consecutive nucleotides in length to SEQ ID NO: 22.
   17. The formulation of any one of embodiments 1-16, wherein the oligonucleotide comprises at least 8 consecutive nucleotides of a nucleotide sequence as set forth in SEQ ID NO: 21, optionally wherein the oligonucleotide comprises the nucleotide sequence of SEQ ID NO: 21.
   18. The formulation of any one of embodiments 2-17, wherein each R¹ comprises a group of the formula (Ib):

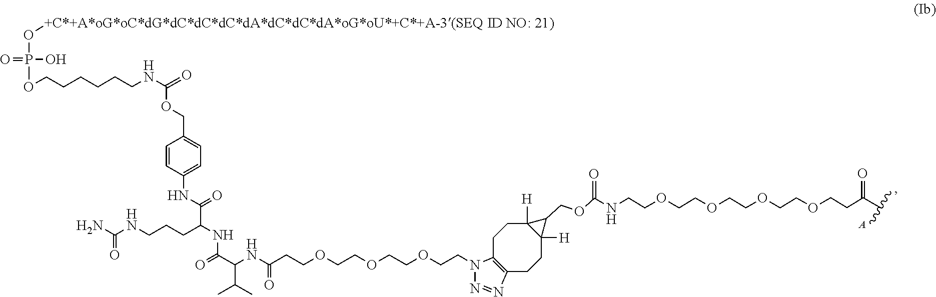

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, such that the oligonucleotide comprises a nucleobase sequence of CAGCGCCCACCAGUCA (SEQ ID NO: 21).
19. The formulation of any one of embodiments 2-17, wherein each R¹ comprises a group of the formula (Ic):
20. The formulation of any one of embodiments 1-19, wherein the complexes are present in the formulation at a concentration in the range of 10 mg/mL to 50 mg/mL.
21. The formulation of any one of embodiments 1-20, further comprising one or more antibodies that are not covalently linked to an oligonucleotide.
22. The formulation of embodiment 21, wherein the average value of n1 of complexes in the formulation is in the range of 0.5 to 5.
23. A method of reducing DMPK expression in a subject, the method comprising administering to the subject an effective amount of the formulation of any one of embodiments 1-22.
24. A method of treating myotonic dystrophy in a subject, the method comprising administering to the subject an effective amount of the formulation of any one of embodiments 1-22.
25. The method of embodiment 23 or embodiment 24, wherein the subject has an expansion of a disease-associated repeat of a DMPK allele that is associated with myotonic dystrophy.
26. The method of embodiment 25, wherein the disease-associated repeat comprises repeating units of a CTG trinucleotide sequence.
27. The method of any one of embodiments 23-26, wherein the complexes reduce DMPK expression in the subject.
28. A complex comprising a structure of formula (I): [R¹]ₙ₁—R², wherein each R¹ comprises a group of the formula (Ia):

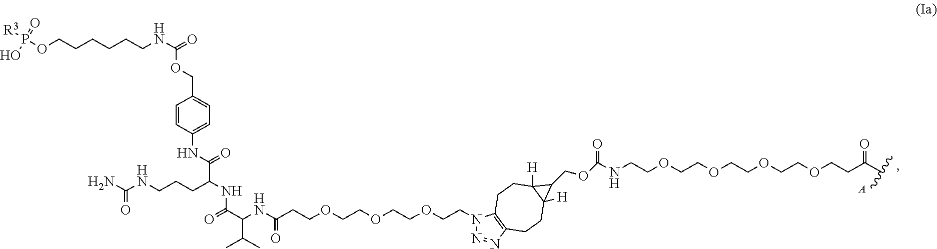

wherein R³ comprises an oligonucleotide comprising a structure of +C*+A*oG*oC*dG*dC*dC*dC*dA*dC*dC*dA*oG*oU*+C*+A (SEQ ID NO: 21), wherein +N represents an LNA (2′-4′ methylene bridge) ribonucleoside, dN represents a 2′-deoxyribonucleoside, oN represents a 2′-MOE modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, and * represents a phosphorothioate internucleoside linkage;
wherein R² comprises a Fab, and wherein the Fab comprises a CDR-H1, a CDR-H2, a CDR-H3, a CDR-L1, a CDR-L2, and a CDR-L3 selected from Table 2,
optionally wherein the Fab comprises a VH comprising the amino acid sequence of SEQ ID NO: 17 and a VL comprising the amino acid sequence of SEQ ID NO: 18,
further optionally wherein the Fab comprises a heavy chain comprising the amino acid sequence of SEQ ID NO: 19 and a light chain comprising the amino acid sequence of SEQ ID NO: 20;
wherein R¹ is covalently linked to R² at attachment point A; and wherein n1 is an integer representing the number of instances of R¹, wherein each instance of R¹ is covalently linked to a different amino acid residue of the Fab, optionally wherein each different amino acid residue is a lysine.
   29. A complex comprising a structure of formula (I): [R¹]ₙ₁—R², wherein each R¹ comprises a group of the formula (Ib):

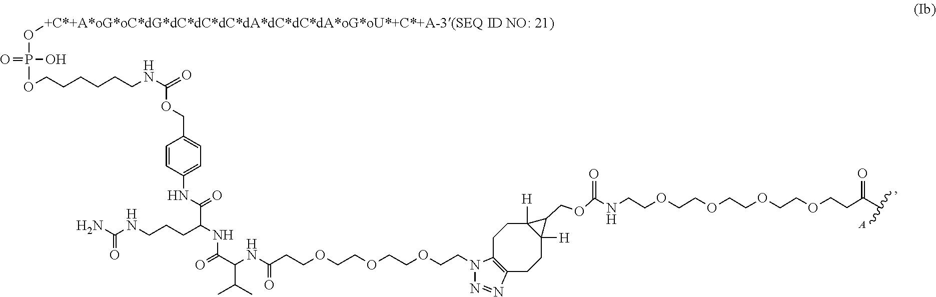

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage, such that the oligonucleotide comprises a nucleobase sequence of CAGCGCCCACCAGUCA (SEQ ID NO: 21);
wherein R² comprises a Fab, and wherein the Fab comprises a CDR-H1, a CDR-H2, a CDR-H3, a CDR-L1, a CDR-L2, and a CDR-L3 selected from Table 2,
optionally wherein the Fab comprises a VH comprising the amino acid sequence of SEQ ID NO: 17 and a VL comprising the amino acid sequence of SEQ ID NO: 18,
further optionally wherein the Fab comprises a heavy chain comprising the amino acid sequence of SEQ ID NO: 19 and a light chain comprising the amino acid sequence of SEQ ID NO: 20;
wherein R¹ is covalently linked to R² at attachment point A; and wherein n1 is an integer representing the number of instances of R¹, wherein each instance of R¹ is covalently linked to a different amino acid residue of the Fab, optionally wherein each different amino acid residue is a lysine.
   30. A complex comprising a structure of formula (I): [R¹]ₙ₁—R², wherein R¹ comprises a group of the formula (Ic):
wherein R² comprises a Fab comprising a CDR-H1, a CDR-H2, a CDR-H3, a CDR-L1, a CDR-L2, and a CDR-L3 selected from Table 2,
optionally wherein R² comprises a Fab comprising a VH comprising the amino acid sequence of SEQ ID NO: 17 and a VL comprising the amino acid sequence of SEQ ID NO: 18,
further optionally wherein R² comprises a Fab comprising a heavy chain comprising the amino acid sequence of SEQ ID NO: 19 and a light chain comprising the amino acid sequence of SEQ ID NO: 20; wherein R¹ is covalently linked to R² at attachment point A;
wherein n1 is an integer representing the number of instances of R¹, wherein each instance of R¹ is covalently linked to a different amino acid residue of the Fab, optionally wherein each different amino acid residue is a lysine.
   31. A complex comprising a structure of the formula (Id):

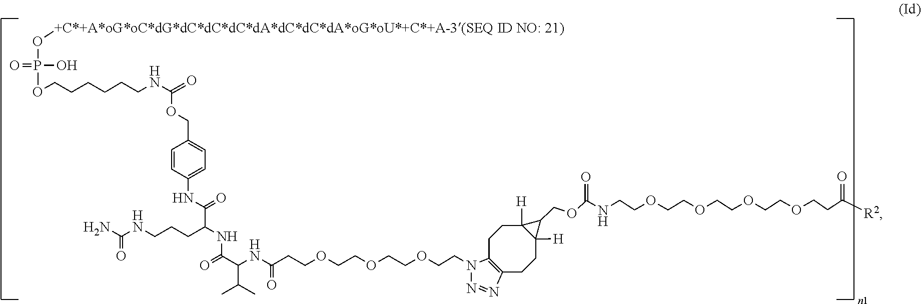

wherein +N represents an LNA (2′-4′ methylene bridge) ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine (2′-4′ methylene bridge), oU represents a 5-methyl-2′-MOE-uridine, * represents a phosphorothioate internucleoside linkage;
wherein R² comprises a Fab, and wherein the Fab comprises a CDR-H1, a CDR-H2, a CDR-H3, a CDR-L1, a CDR-L2, and a CDR-L3 selected from Table 2,
optionally wherein the Fab comprises a VH comprising the amino acid sequence of SEQ ID NO: 17 and a VL comprising the amino acid sequence of SEQ ID NO: 18,
further optionally wherein the Fab comprises a heavy chain comprising the amino acid sequence of SEQ ID NO: 19 and a light chain comprising the amino acid sequence of SEQ ID NO: 20;
wherein n1 is an integer representing the number of instances of the group enclosed by square brackets, wherein each instance of the group enclosed by square brackets is covalently linked to a different amino acid residue of the Fab, optionally wherein each different amino acid residue is a lysine.
   32. A formulation comprising a plurality of complexes of any one of embodiments 28-31, and tris(hydroxymethyl)aminomethane at a concentration of 5 to 50 mM, and sucrose at a concentration of 5 w/v % to 10 w/v %, wherein the formulation is an aqueous solution and is at a pH of 6.5 to 8.5, optionally wherein the plurality of complexes are at a concentration of 10 mg/mL to 50 mg/mL.
   33. A lyophilized form of the formulation of embodiment 32.
   34. A product produced by a process comprising lyophilizing the formulation of embodiment 32.
   35. A frozen form of the formulation of embodiment 32.
   36. A product produced by a process comprising freezing the formulation of embodiment 32.
   37. A formulation comprising a plurality of complexes of any one of embodiments 28-31, and tris(hydroxymethyl)aminomethane at a concentration of 5 to 50 mM, and sucrose at a concentration of 5 w/v % to 15 w/v %, wherein the formulation is an aqueous solution and is at a pH of 6.5 to 8.5, optionally wherein the plurality of complexes are at a concentration of 10 mg/mL to 50 mg/mL.
   38. A formulation comprising a plurality of complexes of any one of embodiments 28-31, and tris(hydroxymethyl)aminomethane at a concentration of 25 mM, and sucrose at a concentration of 10 w/v %, wherein the formulation is an aqueous solution and is at a pH of 7.5, optionally wherein the plurality of complexes are at a concentration of 10 mg/mL to 50 mg/mL.
   39. The formulation of any one of embodiments 32, 37 and 38, further comprising one or more antibodies that are not covalently linked to an oligonucleotide.
   40. The formulation of embodiment 39, wherein the average value of n1 of complexes in the formulation is in the range of 0.5 to 5.
   41. A lyophilized form of the formulation of any one of embodiments 37-40.
   42. A lyophilized cake comprising a plurality of complexes of any one of embodiments 28-31, tris(hydroxymethyl)aminomethane, and sucrose.
   43. The lyophilized cake of embodiment 42, comprising 4.02×10⁻² mg-1.21 mg tris(hydroxymethyl)aminomethane per g of cake; 989 mg-999 mg sucrose per g of cake; and/or 0.666 mg-9.90 mg complexes per g of cake;
optionally wherein the lyophilized cake comprises:
(i) 0.303 mg tris(hydroxymethyl)aminomethane per g of cake, 998.6 mg sucrose per g of cake, and/or 0.999 mg complexes per g of cake;
(ii) 0.302 mg tris(hydroxymethyl)aminomethane per g of cake, 997.7 mg sucrose per g of cake, and/or 1.995 mg complexes per g of cake;
(iii) 0.302 mg tris(hydroxymethyl)aminomethane per g of cake, 997.2 mg sucrose per g of cake, and/or 2.49 mg complexes per g of cake;
(iv) 0.302 mg tris(hydroxymethyl)aminomethane per g of cake, 996.7 mg sucrose per g of cake, and/or 2.99 mg complexes per g of cake;
(v) 0.302 mg tris(hydroxymethyl)aminomethane per g of cake, 995.7 mg sucrose per g of cake, and/or 3.98 mg complexes per g of cake; or
(vi) 0.301 mg tris(hydroxymethyl)aminomethane per g of cake, 994.7 mg sucrose per g of cake, and/or 4.97 mg complexes per g of cake.
   44. The lyophilized cake of embodiment 42 or embodiment 43, wherein when about 10 g of the lyophilized cake are reconstituted in water to produce 1 mL of a solution, the solution comprises tris(hydroxymethyl)aminomethane at a concentration of 5 to 50 mM, sucrose at a concentration of 5 w/v % to 15 w/v %, and complexes at a concentration of 10 mg/mL to 50 mg/mL,
optionally wherein the tris(hydroxymethyl)aminomethane is at a concentration of 25 mM and/or the sucrose is at a concentration of 10 w/v %.

### EQUIVALENTS AND TERMINOLOGY

The disclosure illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the disclosure. Thus, it should be understood that although the present disclosure has been specifically disclosed by preferred embodiments, optional features, modification and variation of the concepts herein disclosed may be resorted to by those skilled in the art, and that such modifications and variations are considered to be within the scope of this disclosure.

In addition, where features or aspects of the disclosure are described in terms of Markush groups or other grouping of alternatives, those skilled in the art will recognize that the disclosure is also thereby described in terms of any individual member or subgroup of members of the Markush group or other group.

It should be appreciated that, in some embodiments, sequences presented in the sequence listing may be referred to in describing the structure of an oligonucleotide or other nucleic acid. In such embodiments, the actual oligonucleotide or other nucleic acid may have one or more alternative nucleotides (e.g., an RNA counterpart of a DNA nucleotide or a DNA counterpart of an RNA nucleotide) and/or (e.g., and) one or more modified nucleotides and/or (e.g., and) one or more modified internucleoside linkages and/or (e.g., and) one or more other modification compared with the specified sequence while retaining essentially same or similar complementary properties as the specified sequence.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. Unless otherwise indicated, ranges of values herein are inclusive of their endpoints (e.g., a range of X to Y is inclusive of the values X and Y). It should be understood that recitations herein of a value from X to Y indicates that the specified value falls in the range of X to Y. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein. Variations of those embodiments may become apparent to those of ordinary skill in the art upon reading the foregoing description.

The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. Those skilled in the art will recognize, or be able to ascertain using no more than routine experimentation, many equivalents to the specific embodiments of the invention described herein. Such equivalents are intended to be encompassed by the following claims.

1-23. (canceled)

24. A complex comprising a structure of formula (I): [R¹]ₙ₁—R², wherein each R¹ comprises a group of the formula (Ia): 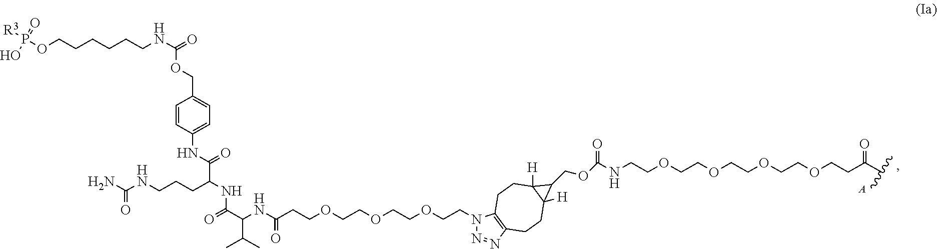
wherein R³ comprises an oligonucleotide comprising a structure of
+C*+A*oG*oC*dG*dC*dC*dC*dA*dC*dC*dA*oG*oU*+C*+A (SEQ ID NO: 21), wherein for each nucleoside, N, of the oligonucleotide, +N represents an LNA ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine, wherein the 2′ and 4′ positions of the sugar are bridged via a methylene, oU represents a 5-methyl-2′-MOE-uridine, and wherein * represents a phosphorothioate internucleoside linkage;
wherein R² comprises a Fab comprising a heavy chain comprising the amino acid sequence of SEQ ID NO: 19 and a light chain comprising the amino acid sequence of SEQ ID NO: 20;
wherein R¹ is covalently linked at attachment point A to R², wherein n1 is an integer representing the number of instances of R¹, and wherein each instance of R¹ is covalently linked to a different amino acid residue of the Fab.

25. The complex of claim 24, wherein each different amino acid residue is a lysine.

26. The complex of claim 24, wherein the heavy chain of the Fab comprises an N-terminal pyroglutamate.

27. A composition comprising the complex of claim 24.

28. The composition of claim 27, wherein the oligonucleotide is in a salt form.

29. The composition of claim 28, wherein the oligonucleotide is in a sodium salt form, potassium salt form, or magnesium salt form.

30. A method of reducing DMPK expression in a subject, the method comprising administering to the subject an effective amount of a composition comprising the complex of claim 24.

31. A method of treating myotonic dystrophy in a subject, the method comprising administering to the subject an effective amount of a composition comprising the complex of claim 24.

32. The method of claim 31, wherein the subject has an expansion of a disease-associated repeat of a DMPK allele that is associated with myotonic dystrophy.

33. The method of claim 32, wherein the disease-associated repeat comprises repeating units of a CTG trinucleotide sequence.

34. A complex comprising a structure of formula (I): [R¹]ₙ₁—R², wherein each R¹ comprises a group of the formula (Ib): 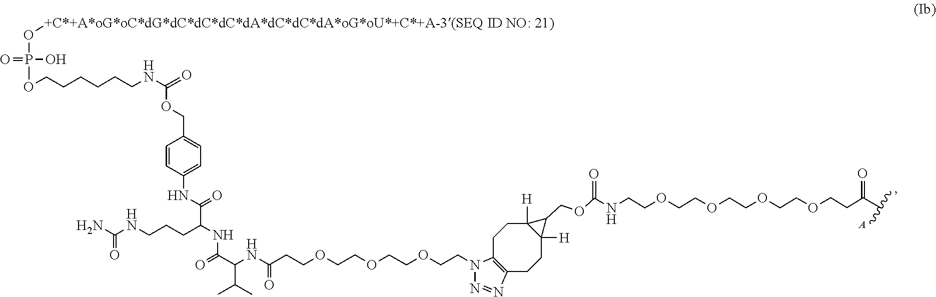
wherein the oligonucleotide of formula (Ib) comprises a sequence of
CAGCGCCCACCAGUCA (SEQ ID NO: 21), wherein for each nucleoside, N, of the oligonucleotide, +N represents an LNA ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine, wherein the 2′ and 4′ positions of the sugar are bridged via a methylene, oU represents a 5-methyl-2′-MOE-uridine, and wherein * represents a phosphorothioate internucleoside linkage;
wherein R² comprises a Fab comprising a heavy chain comprising the amino acid sequence of SEQ ID NO: 19 and a light chain comprising the amino acid sequence of SEQ ID NO: 20;
wherein R¹ is covalently linked at attachment point A to R², wherein n1 is an integer representing the number of instances of R¹, and wherein each instance of R¹ is covalently linked to a different amino acid residue of the Fab.

35. The complex of claim 34, wherein each different amino acid residue is a lysine.

36. The complex of claim 34, wherein the heavy chain of the Fab comprises an N-terminal pyroglutamate.

37. A composition comprising the complex of claim 34.

38. The composition of claim 37, wherein the oligonucleotide is in a salt form.

39. The composition of claim 38, wherein the oligonucleotide is in a sodium salt form, potassium salt form, or magnesium salt form.

40. A method of reducing DMPK expression in a subject, the method comprising administering to the subject an effective amount of a composition comprising the complex of claim 34.

41. A method of treating myotonic dystrophy in a subject, the method comprising administering to the subject an effective amount of a composition comprising the complex of claim 34.

42. The method of claim 41, wherein the subject has an expansion of a disease-associated repeat of a DMPK allele that is associated with myotonic dystrophy.

43. The method of claim 42, wherein the disease-associated repeat comprises repeating units of a CTG trinucleotide sequence.

44. A complex comprising a structure of the formula (Id): 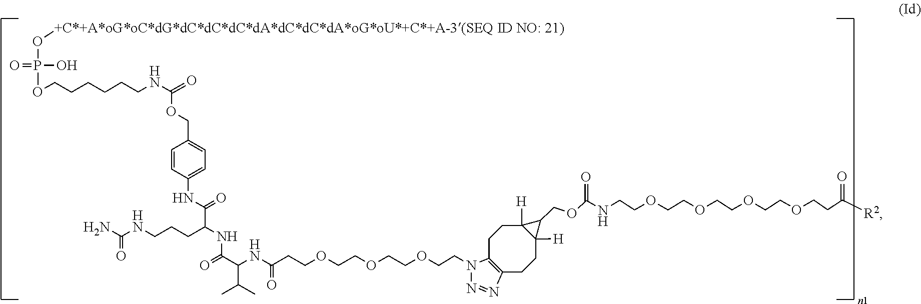
wherein the oligonucleotide of formula (Id) comprises a sequence of
CAGCGCCCACCAGUCA (SEQ ID NO: 21), wherein for each nucleoside, N, of the oligonucleotide, +N represents an LNA ribonucleoside, dN represents a 2′-deoxyribonucleoside, oN represents a 2′-O-methoxyethyl (MOE) modified ribonucleoside, oC represents a 5-methyl-2′-MOE-cytidine, +C represents a 5-methyl-2′-4′-bicyclic-cytidine, wherein the 2′ and 4′ positions of the sugar are bridged via a methylene, oU represents a 5-methyl-2′-MOE-uridine, and wherein * represents a phosphorothioate internucleoside linkage;
wherein R² comprises a Fab comprising a heavy chain comprising the amino acid sequence of SEQ ID NO: 19 and a light chain comprising the amino acid sequence of SEQ ID NO: 20;
wherein n1 is an integer representing the number of instances of the group enclosed by square brackets, wherein each instance of the group enclosed by square brackets is covalently linked to a different amino acid residue of the Fab.

45. The complex of claim 44, wherein each different amino acid residue is a lysine.

46. The complex of claim 44, wherein the heavy chain of the Fab comprises an N-terminal pyroglutamate.

47. A composition comprising the complex of claim 44.

48. The composition of claim 47, wherein the oligonucleotide is in a salt form.

49. The composition of claim 48, wherein the oligonucleotide is in a sodium salt form, potassium salt form, or magnesium salt form.

50. A method of reducing DMPK expression in a subject, the method comprising administering to the subject an effective amount of a composition comprising the complex of claim 44.

51. A method of treating myotonic dystrophy in a subject, the method comprising administering to the subject an effective amount of a composition comprising the complex of claim 44.

52. The method of claim 51, wherein the subject has an expansion of a disease-associated repeat of a DMPK allele that is associated with myotonic dystrophy.

53. The method of claim 52, wherein the disease-associated repeat comprises repeating units of a CTG trinucleotide sequence.
